(19) United States
(12) Patent Application Publication
(10) Pub. No.: US 2007/0036793 A1
(43) Pub. Date: Feb. 15, 2007

(54) Methods for use of an lkb1/strad7mo25 complex

(76) Inventors: David Grahame Hardie, Fife (GB); Dario Alessi, Dundee (GB); Jerome Boudeau, Dundee (GB)

Correspondence Address: ROGALSKY & WEYAND, LLP, P.O. BOX 1927, WILLIAMSVILLE, NY, 14231-1927, US

(21) Appl. No.: 10/565,058

(22) PCT Filed: Jul. 16, 2004
(86) PCT No.: PCT/GB04/03096

§ 371 Date: Jun. 21, 2006

(30) Foreign Application Priority Data
Jul. 17, 2003 (GB) 0316725.1
Dec. 20, 2003 (GB) 0330078.7

(51) Int. Cl. A61K 39/395; G01N 33/53; C12P 21/06; C12N 9/12

(52) U.S. Cl. 424/146.1; 435/7.1; 435/69.1; 435/194; 435/320.1; 435/325; 530/388.26

(57) ABSTRACT

A method for identifying a compound for use in modulating, for example promoting, the activation or phosphorylation of AMPK (AMP-activated protein kinase) or AMPK subfamily member in a cell, the method comprising the steps of (1) determining whether a test compound modulates, for example promotes, the protein kinase activity of LKB1 and (2) selecting a compound which modulates, for example promotes, the protein kinase activity of LKB1. The protein kinase activity may be tested using AMPK or an AMPK subfamily member, or a peptide encompassing the T-loop region of AMPK or an AMPK subfamily member. The LKB1 may be in a preparation or complex with STRAD and/or M025, which has much greater kinase activity than LKB1 in the absence of these accessory proteins. The LKB1, STRAD or M025 may be recombinant. The AMPK subfamily member may be AMPKα1, AMPKα2, NUAK1, NUAK2, BRSK1, BRSK2, SIK, QIK, QSK, MARK1, MARK2, MARK3, MARK4 or MELK.

The present invention relates to protein kinases and their regulation.

LKB1 is a Ser/Thr protein kinase not closely related to other protein kinases (reviewed in (Yoo et al., 2002)). Mutations in LKB1 have been linked to Peutz Jeghers Cancer Syndrome (PJS), an autosomal dominant inherited disorder (Hemminki et al., 1998; Jenne et al., 1998) characterised in those affected by the development multiple gastro-intestinal hamartomatous polyps as well as a wide spectrum of benign and malignant tumours (Hemminki, 1999), with a 15-fold increased risk of developing malignant cancers in many tissues [20, 21]. To date, nearly 100 different mutations in the LKB1 gene have been identified in PJS patients, most of which would be expected to impair the activity of LKB1 [22]. Deletion of both alleles of the LKB1 gene in mice results in embryonic lethality at midgestation arising from multiple defects (Ylikorkala et al., 2001), indicating that LKB1 plays an essential role in development. Importantly LKB1 heterozygous mice are viable but develop a PJS-like disease characterised by gastro-intestinal hamartomas, although it is controversial whether these are caused by haploinsufficiency or loss of heterozygosity (reviewed in [22]). Later on in life (for example over 50 weeks of age) these animals also develop malignant tumours, such as hepatocellular carcinoma which were shown to be associated with loss of LKB1 expression (Bardeesy et al., 2002; Jishage et al., 2002; Miyoshi et al., 2002; Nakau et al., 2002; Rossi et al., 2002). Overexpression of LKB1 in certain cancer cells results in growth suppression due to a G1-cell cycle arrest which is dependent on LKB1 catalytic activity (Tiainen et al., 1999) and this has been proposed to be mediated through activation of p21^{WAF1/CIP1} through a p53-dependent mechanism (Tiainen et al., 2002). Taken together, these findings strongly indicate that LKB1 is likely to function as a tumour suppressor. Recent work has indicated that the C. elegans (Watts et al., 2000), Drosophila (Martin and St Johnston, 2003) and Xenopus (Ossipova et al., 2003) homologues of LKB1 regulate cell polarity and, if this function is conserved in humans, loss of cell polarity in PJS patients could account for the development of hamartomas. Although LKB1 is essentially nuclear when overexpressed in cells, a low level of cytosolic localisation is also frequently observed (Karuman et al., 2001; Smith et al., 1999; Tiainen et al., 1999). Significantly, mutants of LKB1 that are excluded from the nucleus retain full growth suppression activity, suggesting that the cytoplasmic localisation of this enzyme is important for its tumour suppressor function (Tiainen et al., 2002). Several mutant forms of LKB1 found in PJS patients localise only in the nucleus and are not detectable in the cytoplasm (Boudeau et al., 2003b; Tiainen et al., 2002), further suggesting that cytoplasmic localisation of LKB1 is important.

Relatively little is known about how LKB1 is regulated and how it functions. LKB1 is phosphorylated at multiple residues in vivo and mutation of certain phosphorylation sites hinders the ability of LKB1 to suppress cell growth, but the mechanisms by which these phosphorylation events regulate LKB1 function have not yet been defined (Collins et al., 2000; Sapkota et al., 2002a; Sapkota et al., 2002b; Sapkota et al., 2001). There has also been considerable interest in identifying LKB1-interacting proteins, which may regulate its function. To date, a number of proteins have been shown to bind LKB1, namely the p53 tumour suppressor protein (Karuman et al., 2001), the brahma-related gene 1 protein (Brg1), which is a component of chromatin remodelling complexes (Marignani et al., 2001), a protein termed LKB1 interacting protein-1 (LIP1) (Smith et al., 2001) and the kinase-specific chaperone complex consisting of Cdc37 and Hsp90 (Boudeau et al., 2003a). The binding of LKB1 to p53 was proposed to be essential for mediating p53-dependent cell death (Karuman et al., 2001), whereas the binding of LKB1 to Brg1 was suggested to be required for Brg1-dependent cell growth arrest (Marignani et al., 2001). The interaction of LKB1 with LIP1 anchored LKB1 in the cytoplasm (Smith et al., 2001), while the binding of LKB1 to Cdc37 and Hsp90 stabilised the LKB1 protein in cells (Boudeau et al., 2003a).

Recently, we have shown that LKB1 is associated with a STE20-related pseudokinase termed STRADα (STe20-Related ADaptor protein-α), which lacks catalytic activity because key residues required for the function of nearly all protein kinases are missing (Baas et al., 2003). Moreover, LKB1 binds STRADα through its catalytic domain and this interaction enhances LKB1 in vivo activity as well as promoting LKB1 cytoplasmic localisation (Baas et al., 2003). LKB1 is no longer able to suppress cell growth in cells in which STRADα has been depleted using an siRNA approach, suggesting that the binding of LKB1 to STRADα plays an important role in mediating its tumour suppressor function. STRADα is also phosphorylated in vitro and in vivo by LKB1, making STRADα the first physiological substrate for LKB1 identified thus far (Baas et al., 2003).

AMP-activated protein kinase kinase (AMPKK) and AMP-activated protein kinase (AMPK) are the upstream and downstream components of a protein kinase cascade that acts as a sensor of cellular energy charge [1,2]. AMPK is activated by an elevation, in cellular 5′-AMP that always accompanies a fall in the ATP:ADP ratio due to the reaction catalysed by adenylate kinase (2ADP [symbol] ATP+ADP). This occurs during metabolic stresses such as hypoxia, ischaemia, glucose deprivation and, in skeletal and cardiac muscle, during contraction or exercise [1-3]. This applies to both catalytic subunit isoforms of AMPK (AMPKα1 and AMPKα2). Once activated by stress, AMPK switches on glucose and fatty acid uptake and oxidative metabolism of these fuels to generate ATP, while switching off biosynthetic pathways that consume ATP. It achieves this metabolic switching both by direct phosphorylation of metabolic enzymes and by longer-term effects on gene expression [1, 2]. AMPK is also activated by metformin, one of the most widely used diabetes drugs (though required at high doses ie >2 g per day in adults), by an unknown mechanism that does not involve lowering of cellular ATP levels. The activation of AMPK by both ATP-depletion and phenformin requires phosphorylation of the AMPK catalytic (a) subunit at its T-loop residue (Thr 172 in AMPKα1) by an upstream kinase. While AMPK is best known for its effects on metabolism, recent work suggests that it also regulates cell growth and proliferation. Firstly, activation of the kinase inhibits protein synthesis by causing phosphorylation of elongation factor-2 [4], and by inhibiting phosphorylation of p70S6K by the mTOR pathway [5,6]. Secondly, it has both pro-apoptotic effects [7-9] and anti-apoptotic effects [10, 11] in different cells. Thirdly, it reduces the level of the RNA binding protein HuR in the cytoplasm, decreasing the stability of mRNAs encoding proliferative genes such as cyclins A and B1 [12]. Fourthly, it inhibits proliferation of HepG2 cells by stabilizing p53 [13].

We have previously partially purified from rat liver an upstream kinase, AMPKK, that activates AMPK by phosphorylation at Thr-172 within the activation loop of the kinase domain [14]. However, we have been unable to purify enough material to obtain amino acid sequence and identify the activity as a defined gene product. We searched for kinases upstream of the Saccharomyces cerevisiae homologue of AMPK (the SNF1 complex), by assaying the ability of a collection of 119 yeast protein kinase-glutathione-S-transferase (GST) fusions to activate the mammalian and yeast kinases. This approach identified the Elm1 protein kinase as a potential upstream kinase [15]. By analysing protein kinases that interacted with Snf1 by two hybrid analysis, Schmidt and colleagues identified Pak1 as another potential upstream kinase in yeast [16]. Neither elm1Δ nor pak1Δ strains, in which the single kinase genes were deleted, had the same phenotype as a snf1Δ strain. However, Elm1 and Pak1 form a small sub-family with the Tos3 protein kinase, and a triple elm1Δ pak1Δ tos3Δ mutant strain had phenotype similar to snf1Δ that was restored to wild type by expression of any one of the three kinases [15]. This proved that these three protein kinases act as upstream kinases for the SNF1 complex in a partially redundant manner.

The nearest relatives of Elm1, Pak1 and Tos3 encoded by the human genome are the β isoform of calmodulin-dependent protein kinase kinase (CaMKKβ). We have previously shown that a CaMKK purified from pig brain could activate AMPK in cell-free assays, albeit rather poorly in comparison to the activation of calmodulin-dependent protein kinase I (CAMKI). However, the AMPKK previously purified from rat liver was not calmodulin-dependent [17]. Amongst other less closely related relatives to the Elm1/Pak1/Tos3 family in humans is LKB1 (see alignment in FIG. 1), a 50 kDa serine/threonine kinase that was originally discovered as the gene mutated in the inherited disorder Peutz-Jeghers syndrome (PJS) [18, 19], as discussed above. Several human tumour cell lines, including HeLa and G361 (melanoma) cells, lack expression of LKB1 mRNA and protein, and expression of wild type LKB1 in these cells caused a growth arrest in G1 phase, whereas expression of catalytically inactive or several LKB1 mutations isolated from PJS mutations failed to arrest cell growth [23]. Growth arrest induced by expression of LKB1 in G361 cells was also reported to be reversed by co-expression of cyclin D1 or E, was associated with the transcriptional induction of the cyclin-dependent kinase inhibitor p21^{WAF1/CIP1}, and was shown to be dependent on p53 [24]. Taken together, these results show that LKB1 acts as a tumour suppressor and that the catalytic activity of LKB1 is essential for this function. However, the downstream substrate(s) that LKB1 phosphorylates to mediate the suppression of cell growth remained unknown.

We identify MO25α (MOuse protein 25-α) as a novel component of the LKB1-STRADα complex and establish that MO25α plays an important role in stabilising this complex in the cell cytoplasm as well as enhancing LKB1 catalytic activity. For each of STRADα and MO25α there is also a closely related isoform (STRADβ and MO25β) encoded in the human genome, which can also associate with LKB1. We demonstrate that MO25 may function as a scaffolding component of the LKB1-STRAD complex. Although STRADα [30] and STRADβ are related to the Ste20 protein kinases, several of the residues expected in an active protein kinase are not conserved, and they are therefore classified as “pseudokinases”. MO25α (for example) binds to the C-terminus of, for example, STRADα and stabilizes the association between STRADα and LKB1. The association of LKB1 with STRAD and MO25 increases the kinase activity against an artificial substrate (myelin basic protein) and also enhances its cytoplasmic localization, which was previously implicated in the tumour suppressor function of LKB1, since mutants lacking a nuclear localisation signal were still capable of suppressing cell growth [24].

Further, we describe the resolution from rat liver of two upstream kinases (AMPKK1 and AMPKK2). We show that AMPKK1 and AMPKK2 both contain LKB1 complexed with STRADα and MO25α, and that both activities are immunoprecipitated with anti-LKB1 antibodies. Moreover, both endogenous and recombinant LKB1/STRAD/MO25 complexes isolated from HEK-293T cells activate AMPK via phosphorylation of Thr-172 on the α subunit. We demonstrate that the ability of LKB1 to phosphorylate and activate AMPK is enhanced dramatically by the presence of STRAD and MO25 subunits, suggesting that both subunits are essential for maximal activity. Finally, drugs that activate AMPK in other cell types (AICA riboside and phenformin), did not activate the AMPK in HeLa cells, which do not express LKB1. However, the stable expression of wild type LKB1, but not a catalytically-inactive mutant, restored activation of AMPK by these drugs. Accordingly, we show that the LKB1, in complex with STRAD and MO25, is the physiological activator of AMPK. It is now possible to perform screens for identifying compounds for modulating the physiological activation of AMPK.

A first aspect of the invention provides a method for identifying a compound for use in modulating, for example promoting, the activation or phosphorylation of AMPK (AMP-activated protein kinase) or AMPK subfamily member in a cell, the method comprising the steps of (1) determining whether a test compound modulates, for example promotes, the protein kinase activity of LKB1 and (2) selecting a compound which modulates, for example promotes, the protein kinase activity of LKB1.

The protein kinase activity of LKB1 that is modulated/assessed in the screening method may be phosphorylation of a non-physiological substrate such as myelin basic protein (for example as discussed in the Examples). Alternatively, the protein kinase activity of LKB1 that is modulated/assessed in the screening method may be phosphorylation of AMPK or an AMPK subfamily member, or fragment either thereof, as discussed further. Phosphorylation of AMPK or an AMPK subfamily member may be assessed by measuring activation or phosphorylation of the AMPK or AMPK subfamily member, as discussed further below and in the Examples. For example, suitable peptide substrates for LKB1 are discussed in Example 3 and 4 and include the NUAK2 T-loop peptide (termed LKBtide). AMPK activation (for example) may be assessed using a reporter gene whose expression is modulated by AMPK. Other techniques and reagents that may be useful in measuring LKB protein kinase activity and/or phosphorylation or activation of AMPK or an AMPK subfamily member (or fragment thereof are described in the Examples, for example in Example 4.

The protein kinase activity may be increased or reduced by an alteration in the Vₘₐₓ or the Kₘ (or both) of the LKB1 (or AMKP or subfamily member, as appropriate) for a particular substrate. For example, activity may be increased by an increased Vₘₐₓ or decreased Kₘ. It will be appreciated that it may not be necessary to determine the value of either Vₘₐₓ or Kₘ in order to determine whether the LKB1 (or AMKP or subfamily member, as appropriate) has been activated or deactivated. It will be appreciated that dephosphorylated (deactivated) AMPK or subfamily member may retain some enzymatic activity.

Activity may be measured as the amount of a substrate phosphorylated in a given time; a change of activity may therefore be detected as a change in the amount of substrate (for example, at a single concentration) that is phosphorylated in a given time. It is preferred that the activity is increased or decreased, as appropriate, by at least 2, preferably 5, 10, 15, 20, 25, 30 or 50-fold.

It will be appreciated that it may be necessary to determine the effect of the compound on the activity of the substrate (for example AMPK), for example by measuring the activity of the substrate when exposed to the compound (1) after exposure of the substrate to LKB1, (2) before exposure of the substrate to LKB1 and/or (3) without exposure to LKB1.

Expression of a protein encoded by an RNA transcribed from a promoter regulated (directly or indirectly) by a substrate of LKB1 (or production of the RNA itself) may be measured. The protein may be one that is physiologically regulated by a substrate of LKB1, for example AMPK or subfamily member, or may be a “reporter” protein, as well known to those skilled in the art (ie a recombinant construct may be used). A reporter protein may be one whose activity may easily be assayed, for example β-galactosidase, chloramphenicol acetyltransferase or luciferase (see, for example, Tan et al (1996)).

AMPK activation, for example in the liver, decreases the expression of enzymes of gluconeogenesis (phosphenolpyruvate carboxylase and glucose-6-phosphatase); see Lochhead et al 5-aminoimidazole-4-carboxamide riboside mimics the effects of insulin on the expression of the 2 key gluconeogenic genes PEPCK and glucose-6-phosphatase. Diabetes. 2000 June; 49(6):896-903; Yamauchi et al Adiponectin stimulates glucose utilization and fatty-acid oxidation by activating AMP-activated protein kinase. Nat Med. 2002 November; 8(11):1288-95. Another class of genes that are switched off by AMPK, for example in the liver, are those encoding enzymes of fatty acid biosynthesis (fatty acid synthase and AcetylCoa carboxylase); see Woods et al Characterization of the role of AMP-activated protein kinase in the regulation of glucose-activated gene expression using constitutively active and dominant negative forms of the kinase. Mol Cell Biol. 2000 September; 20(18):6704-11. AMPK activation also reduces the level of 4 transcription factors: Sterol response element binding protein-1C (Zhou et al Role of AMP-activated protein kinase in mechanism of metformin action. J Clin Invest. 2001 October; 108(8):1167-74.); hepatocyte nuclear factor-4alpha (Leclerc et al Hepatocyte nuclear factor-4alpha involved in type 1 maturity-onset diabetes of the young is a novel target of AMP-activated protein kinase. Diabetes. 2001 July; 50(7):1515-21); C/EBPalpha and PPAR-gamma (Habinowski et al The effects of AICAR on adipocyte differentiation of 3T3-LI cells. Biochem Biophys Res Commun. 2001 Sep. 7; 286(5):852-6). Accordingly, such genes (or recombinant genes incorporating regulatory sequences from these genes) may be used as reporter genes.

By modulation of the protein kinase activity is included inhibition or an increase in the protein kinase activity.

It will be appreciated that in the methods of the invention wherein phosphorylation of a polypeptide may occur that the presence of a suitable phosphate donor may be required, as described for the above aspect of the invention. Suitable phosphate donors will be known to those skilled in the art and include ATP, for example as the magnesium salt (MgATP), as described in the Examples.

The LKB1 is preferably in a preparation with STRAD and/or MO25. As noted above, LKB1 is considered to be present in a complex with these polypeptides in cells and the presence of these polypeptides is considered to enhance LKB1 activity. The LKB1 (and preferably also STRAD and/or MO25) may, for example, be purified from cells in which the LKB1 (and preferably also STRAD and/or MO25) are expressed naturally, but it may be more convenient for at least one of the LKB1, STRAD or MO25 to be recombinant.

The term AMPK will be well known to those skilled in the art, as indicated above. The AMPK (or other substrate, for example myelin basic protein) used in the assay may be recombinant or non-recombinant. The AMPK may be a bacterially-expressed AMPKα1 catalytic domain (for example as described in [44]) or a heterotrimeric complex such as those described in [45] and in Example 2. The AMPK may have the amino acid sequence of a naturally occuring AMPK, or may be or comprise a fusion polypeptide (for example as described in [45] or Example 2), or may be a fragment or variant of a naturally occuring AMPK that retains the ability to be phosphorylated or activated by LKB1, for example as described in Example 2. Thus, it is preferred that the AMPK is an AMPK that retains a threonine (or serine) residue at the position equivalent to Threonine172 of full length wild-type human α1 catalytic domain. It is preferred that the AMPK is not a mutant in which Thr172 is replaced by a residue other than serine, for example is replaced by alanine. A fragment derivable from AMPK (or from an AMPK subfamily member) which encompasses the Thr172 residue and at least part of the T-loop sequence which includes this residue, for example at least the 2, 3, 4, 5, 6 or 7 residues C-terminal and N-terminal of this residue, is a suitable substrate for use in the screening method. An example of such a peptide substrate is described in Example 3.

Examples of Accession GI numbers for catalytic domain of human AMPK alpha 1 subunit include:

>gi|5453964|ref|NP_{—}006242.1| LocusLink info protein kinase, AMP-activated, alpha 1 catalytic subunit; AMPK alpha 1; Protein kinase, AMP-activated, catalytic, alpha-1 [Homo sapiens]

gi|20178277|sp|Q13131|AAK1_HUMAN 5′-AMP-activated protein kinase, catalytic alpha-1 chain (AMPK alpha-1 chain)

gi|4115829|dbj|BAA36547.1| LocusLink info AMP-activated protein kinase alpha-1 [Homo sapiens]

Examples of Accession GI numbers for catalytic domain of human AMPK alpha 2 subunit include:

>gi|11493357|emb|CAC17574.1| dJ758N20.1 (protein kinase, AMP-activated, alpha 2 catalytic subunit) [Homo sapiens]

>gi|5453966|ref|NP_{—}006243.1| LocusLink info protein kinase, AMP-activated, alpha 2 catalytic subunit; protein kinase, AMP-activated [Homo sapiens]

The term LKB1 will similarly be well known to those skilled in the art. The LKB1 used in the assay may be recombinant or non-recombinant. The LKB1 may be bacterially expressed, but it is preferred that it is expressed in a mammalian system and/or expressed alongside STRAD and/or MO25, preferably both, for example as described in Examples 1 and 2. The LKB1 may have the amino acid sequence of a naturally occuring LKB1, or may be a fusion polypeptide (for example as described in the Examples), or may be a fragment or variant of a naturally occuring LKB1 that retains the ability to phosphorylate or activate AMPK, for example on Thr172 of AMPK, for example as described in Example 2. Thus, the LKB1 is an LKB1 that retains an active kinase domain. A fragment of LKB1 which contains the intact kinase domain but not other regions of LKB1 may be useful; this region of LKB1 is sufficient to retain protein kinase activity and to interact with STRAD. The LKB1 used in the assay is not a kinase-dead mutant such as is described in the Examples. It is preferred that the LKB1 retains the ability to interact with STRAD and/or MO25, as discussed further below.

Accession numbers for LKB1 include the following:

AAC15742 Peutz-Jeghers syndrome protein [Homo sapiens] gi|3063585|gb|AAC15742.1|[3063585]

Q15831 Serine/threonine protein kinase 11 (Serine/threonine-protein kinase LKB1) gi|3024670|sp|Q15831|STKB_HUMAN[3024670]

NP_{—}000446 serine/threonine protein kinase 11 [Homo sapiens] gi|4507271|ref|NP_{—}000446.1|[4507271]

It is particularly preferred, although not essential, that the LKB1 polypeptide has at least 30% of the enzyme activity of full-length human LKB1 with respect to the phosphorylation of full-length human AMPKα1 on residue Thr172 (preferably in the presence of STRAD and MO25) or a peptide substrate encompassing this region, or phosphorylation of myelin basic protein. It is more preferred if the LKB1 polypeptide has at least 50%, preferably at least 70% and more preferably at least 90% of the enzyme activity of full-length human LKB1 with respect to the phosphorylation of full-length human AMPKα1 on residue Thr172 (preferably in the presence of STRAD and MO25) or a peptide substrate encompassing this region.

Similarly, the terms STRAD or MO25 will similarly be well known to those skilled in the art. For example, a STRAD polypeptide (STRADα) is described in Baas et al (2003) EMBO J 22(12), 3062-3072 and in EMBL/GenBank Accession No AF308302. Human STRADα and STRADβ (NCBI accession number AAM19143) sequences are shown in FIG. 10. MO25 polypeptides are described in Miyamoto et al (1993); Karos & Fisher (1999) and Nozaki et al (1996) and, for example, in Accession No NP_{—}057373 (human MO25α) and Q9H9S4 (human MO25β). Further examples of MO25 polypeptides are shown in FIG. 2 and further Accession Nos are given in the figure legend. MO25 bears no sequence homology to other proteins in the database but recent studies indicate that it is structurally related to the Armadillo repeat domain (Milburn et al., 2003). The STRAD or MO25 used in the assay may be recombinant or non-recombinant. The STRAD or MO25 may be bacterially expressed, but it is preferred that they are expressed in a mammalian system and/or expressed alongside LKB1, for example as described in Examples 1 and 2. The STRAD or MO25 may have the amino acid sequence of a naturally occuring STRAD or MO25, or may be a fusion polypeptide (for example as described in the Examples), or may be a fragment or variant of a naturally occuring STRAD or MO25 that retains the ability for the STRAD to bind to the MO25 and to LKB1, and for the MO25 to bind to the STRAD or complex of LKB1 and STRAD. At least the C-terminal region of MO25 may be required. In view of the sequence conservation throughout the length of MO25 it is considered that it may be desirable to use full length MO25. It is preferred that the STRAD polypeptide has the C-terminal sequence Trp-Asp/Glu-Phe (which MO25 is considered to recognise) but this is not considered to be essential, because MO25 can bind to STRAD lacking these residues when the STRAD is bound to LKB1 (see Example 1). It is also preferred that the pseudokinase domain of STRAD is present, as this may be required for MO25 binding. For example, it is preferred that the STRAD is not a fragment lacking the residues corresponding to the C-terminal 88 amino acids of full length STRADα (which has a truncated pseudokinase domain and does not interact with human LKB1 or MO25α). It may be desirable to use full length STRAD.

The capability of, for example, the said polypeptide with regard to interacting with or binding to, for example, a STRAD polypeptide, may be measured by any method of detecting/measuring a protein/protein interaction, as discussed further below. Suitable methods include yeast two-hybrid interactions, co-purification, ELISA, co-immunoprecipitation, cross-linking studies, structural studies, fluorescence resonance energy transfer (FRET) methods and surface plasmon resonance methods. Thus, the said MO25 (for example human MO25α) may be considered capable of binding to or interacting with a STRAD polypeptide (for example human STRADα) if an interaction may be detected between the said MO25 polypeptide and the said STRAD polypeptide by ELISA, co-immunoprecipitation or surface plasmon resonance methods or by a yeast two-hybrid interaction or copurification method, for example as described in Example 1 or Example 2.

It is preferred that the interaction can be detected using a surface plasmon resonance method, as described in, for example WO 00/56864. One polypeptide may be immobilised on the test surface, for example it can be coupled through amino groups to a Sensor Chip CM5™, according to the manufacturer's instructions, or a biotinylated polypeptide can be bound to an avidin coated SensorChip SA. The other polypeptide (at concentrations between, for example 0 and between 10 μM and 1.0 μM, for example 2 μM) is then injected over the surface and steady state binding determined in each case. From these measurements a K_{d} can be determined. It is preferred that the interaction has a K_{d} of less than 8 μM, more preferably less than 5 μM, 2 μM, 1 μM, 500 nM, 300 nM, 200 nM or 100 nM, for example about 150 nM. Alternatively, a K_{d} can be determined for a polypeptide in competition with the immobilised polypeptide. One polypeptide (for example at a concentration of 0.5 μM) is mixed with free polypeptide (for example, at concentrations between 0 and 3 μM) and the mixture injected over the immobilised polypeptides. The steady state binding is determined in each case, from which the K_{d} of the interaction can be determined using the Cheng-Prescott relationship. Alternatively, the interaction may be expressed in terms of an observed response or relative observed responses, measured in terms of mass of protein bound to the surface.

By “variants” of a polypeptide we include insertions, deletions and substitutions, either conservative or non-conservative. In particular we include variants of the polypeptide where such changes do not substantially alter the protein kinase activity or ability to bind to particular binding partners, as appropriate.

By “conservative substitutions” is intended combinations such as Gly, Ala; Vat, lie, Leu; Asp, Glu; Asn, Gln; Ser, Thr; Lys, Arg; and Phe, Tyr.

The three-letter or one letter amino acid code of the IUPAC-IUB Biochemical Nomenclature Commission is used herein, with the exception of the symbol Zaa, defined above. In particular, Xaa represents any amino acid. It is preferred that at least the amino acids corresponding to the consensus sequences defined herein are L-amino acids.

It is particularly preferred if the polypeptide variant has an amino acid sequence which has at least 65% identity with the amino acid sequence of the relevant human polypeptide, more preferably at least 70%, 71%, 72%, 73% or 74%, still more preferably at least 75%, yet still more preferably at least 80%, in further preference at least 85%, in still further preference at least 90% and most preferably at least 95% or 97% identity with the amino acid sequence of the relevant human polypeptide.

It is still further preferred if a protein kinase variant has an amino acid sequence which has at least 65% identity with the amino acid sequence of the catalytic domain of the human polypeptide, more preferably at least 70%, 71%, 72%, 73% or 74%, still more preferably at least 75%, yet still more preferably at least 80%, in further preference at least 83 or 85%, in still further preference at least 90% and most preferably at least 95% or 97% identity with the relevant human amino acid sequence.

It will be appreciated that the catalytic domain of a protein kinase-related polypeptide may be readily identified by a person skilled in the art, for example using sequence comparisons as described below. Protein kinases show a conserved catalytic core, as reviewed in Johnson et al (1996) Cell, 85, 149-158 and Taylor & Radzio-Andzelm (1994) Structure 2, 345-355. This core folds into a small N-terminal lobe largely comprising anti-parallel β-sheet, and a large C-terminal lobe which is mostly α-helical.

The percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally.

The alignment may alternatively be carried out using the Clustal W program (Thompson et al., 1994). The parameters used may be as follows:

Fast pairwise alignment parameters: K-tuple (word) size; 1, window size; 5, gap penalty; 3, number of top diagonals; 5. Scoring method: x percent.

Multiple alignment parameters: gap open penalty; 10, gap extension penalty; 0.05.

Scoring matrix: BLOSUM.

It will be appreciated that the MO25 sequences shown in FIG. 2 are examples of MO25 variants as defined above.

The residue equivalent to, for example, Thr 172 of full-length human AMPKα1 may be identified by alignment of the sequence of the polypeptide with that of full-length human AMPKα1 in such a way as to maximise the match between the sequences. The alignment may be carried out by visual inspection and/or by the use of suitable computer programs, for example the GAP program of the University of Wisconsin Genetic Computing Group, which will also allow the percent identity of the polypeptides to be calculated. The Align program (Pearson (1994) in: Methods in Molecular Biology, Computer Analysis of Sequence Data, Part II (Griffin, AM and Griffin, HG eds) pp 365-389, Humana Press, Clifton). Thus, residues identified in this manner are also “equivalent residues”.

It will be appreciated that in the case of truncated forms of AMPKα1 or in forms where simple replacements of amino acids have occurred it is facile to identify the “equivalent residue”.

It is preferred that the polypeptides used in the screen are mammalian, preferably human (or a species useful in agriculture or as a domesticated or companion animal, for example dog, cat, horse, cow), including naturally occurring allelic variants (including splice variants). The polypeptides used in the screen may comprise a GST portion or may be biotinylated or otherwise tagged, for example with a 6His, HA, myc or other epitope tag, as known to those skilled in the art, or as described in Examples 1 and 2. This may be useful in purifying and/or detecting the polypeptide(s).

The effect of the compound may be determined by comparing the rate or degree of phosphorylation of the substrate polypeptide by the LBK1 in the presence of different concentrations of the compound, for example in the absence and in the presence of the compound, for example at a concentration of about 100 μM, 30 μM, 10 μM, 3 μM, 1 μM, 0.1 μM, 0.01 μM and/or 0.001 μM.

The compound may mimic the effect of the interaction of STRAD and/or MO25 with LKB1. A compound that mimics the effect of STRAD and/or MO25 on LKB1 may increase the rate or extent of phosphorylation of the substrate polypeptide (for example AMPK or a subfamily member or a fragment any thereof).

By “mimic the effect of the interaction of STRAD and/or MO25 with LKB1” is meant that the compound has a quantitative or qualitative effect on the LKB1, for example on its protein kinase activity or stability, that is the same as an effect of the STRAD or MO25 on the protein kinase, for example on its protein kinase activity or stability, as discussed in Examples 1 and 2. For example, STRAD and MO25 are considered to increase the rate at which LKB1 phosphorylates, for example, AMPK or myelin basic protein; a mimic of STRAD or MO25 may increase the rate at which LKB1 (in the absence of STRAD and/or MO25) phosphorylates a substrate polypeptide, for example AMPK.

The AMPK subfamily is discussed in Example 2 (and see also [41]) and includes the following polypeptides:

MELK (Heyer et al., 1999 Expression of Melk, a new protein kinase, during early mouse development. Dev Dyn, 215, 344-351; Heyer et al., 1997 New member of the Snf1/AMPK kinase family, Melk, is expressed in the mouse egg and preimplantation embryo. Mol Reprod Dev, 47, 148-156.)

QIK (Xia et al., 2000 The new serine-threonine kinase, Qik, is a target of the Qin oncogene. Biochem Biophys ResCommun, 276, 564-570)

SIK (Coyle et al., 2003 Sam68 enhances the cytoplasmic utilization of intron-containing RNA and is functionally regulated by the nuclear kinase Sik/BRK. Mol Cell Biol, 23, 92-103; Derry et al., 2003 Altered localization and activity of the intracellular tyrosine kinase BRK/Sik in prostate tumor cells. Oncogene, 22, 4212-4220; Derry et al., 2000 Sik (BRK) phosphorylates Sam68 in the nucleus and negatively regulates its RNA binding ability. Mol Cell Biol, 20, 6114-6126; Feldman et al., 2000 The salt-inducible kinase, SIK, is induced by depolarization in brain. J Neurochem, 74, 2227-2238; Horike et al., 2002 Roles of several domains identified in the primary structure of salt-inducible kinase (SIK). Endocr Res, 28, 291-294.; Lin et al., 2000 SIK (Salt-inducible kinase): regulation of ACTH-mediated steroidogenic gene expression and nuclear/cytosol redistribution. Endocr Res, 26, 995-1002.; Llor et al., 1999 BRK/Sik expression in the gastrointestinal tract and in colon tumors. Clin Cancer Res, 5, 1767-1777.; Vasioukhin and Tyner, 1997 A role for the epithelial-cell-specific tyrosine kinase Sik during keratinocyte differentiation. Proc Natl Acad Sci USA, 94, 14477-14482.; Wang et al., 1999 Cloning of a novel kinase (SIK) of the SNF1/AMPK family from high salt diet-treated rat adrenal. FEBS Lett, 453, 135-139)

Other family members include NuaK1, NuaK2, BrsK1, BrsK2 and QSK, about which little is known. We consider that LKB1 may phosphorylate and activate these other protein kinases in the AMPK sub-family.

Other family members (which lie on an immediately adjacent branch of the kinase tree to the family members indicated above) include MARK1, MARK2, MARK3 and MARK4. See, for example, Manning et al (2002) The protein kinase complement of the human genome Science 298, 1912-1934. MARK3 is also known as PARIA or C-TAK1 (Peng et al (1998) Cell Growth Differ 9, 197-208; Spicer et al (2003) Oncogene 22, 4752-4756.

Metformin or its analogues may be acting through activation of one of these kinases as well as through AMPK. Such a sub-family member or fragment thereof or fusion of such a fragment (for example a T-loop peptide, as discussed above and in Example 3) may be a suitable substrate for use in the screening method of the first aspect of the invention.

A compound identified by a method of the invention may modulate the ability of the protein kinase to phosphorylate different substrates, for example different naturally occuring members of the AMPK subfamily, to different extents. Thus, it is preferred, but not essential, that when screening for a compound for use in modulating AMPK activity that AMPK or a fragment thereof is used as the substrate. Similarly, it is preferred, but not essential, that when screening for a compound for use in modulating the activity of a particular AMPK subfamily member that that subfamily member or a fragment thereof is used as the substrate.

The method is useful in identifying compounds that, for example, promote the activation of AMPK or a sub-family member. A compound that triggers the activation of AMPK or an AMPK sub-family member may be useful in the treatment of diabetes and obesity. Such a compound may mimic the activity of metformin or its analogues and may be of higher potency.

LKB1 is also a tumour suppressor protein kinase that inhibits cell growth and proliferation. The results presented herein indicate that LKB1's tumour suppressor functions may be mediated through LKB1 phosphorylating and triggering the activation of AMPK and/or one or more subfamily members indicated above. Thus, these polypeptides may be novel tumour suppressor protein kinases and a compound which activates one or more of these protein kinases may be useful for treating cancer.

A compound which promotes phosphorylation or activation of AMPK or an AMPK family member may act by stabilising the LKB1-STRAD-MO25 complex. It may be useful to screen for such a stabilising effect (either before or after or instead of selection on the basis of a protein kinase activity assay as described above). For example, such a screen could involve assessing the effects of a compound on complex formation or stability, for example using methods for assessing molecular interactions, as discussed above. For example, coimmunoprecipitation or copurification methods may be used, or a reporter gene/yeast two hybrid system may be used. A complex may also be detected using fluorescence resonance energy transfer (FRET) techniques (for example using fusion proteins comprising fluorescent proteins, for example green, blue or yellow fluorescent proteins (GFPs; YFPs, BFPs, as well known to those skilled in the art)), for example in material from cells in which the LKB1 and the STRAD and/or MO25 are coexpressed, as described in Examples 1 and 2.

The compound may be one which binds to or near a region of contact between two or more of LKB1, STRAD and MO25, or may be one which binds to another region and, for example, induces a conformational or allosteric change which stabilises (or destabilises) the complex; or promotes (or inhibits) its formation. The compound may bind to LKB1 or STRAD or MO25 (or to the complex as a whole) so as to increase the LKB1 protein kinase activity by an allosteric effect. This allosteric effect may be an allosteric effect that is involved in the natural regulation of LKB1's activity.

A further aspect of the invention provides a purified preparation comprising LKB1, STRAD and recombinant MO25. The preparation may comprise recombinant LKB1 and/or recombinant STRAD. The preparation may be useful in an assay of the first aspect of the invention.

By “purifed” is meant that the preparation has been at least partially separated from other components in the presence of which it has been formed, for example other components of a recombinant cell. Examples of methods of purification than may be used are described in the Examples.

The preparation may be substantially pure. By “substantially pure” we mean that the said polypeptide(s) are substantially free of other proteins. Thus, we include any composition that includes at least 30% of the protein content by weight as the said polypeptides, preferably at least 50%, more preferably at least 70%, still more preferably at least 90% and most preferably at least 95% of the protein content is the said polypeptides.

Thus, the invention also includes compositions comprising the said polypeptides and a contaminant wherein the contaminant comprises less than 70% of the composition by weight, preferably less than 50% of the composition, more preferably less than 30% of the composition, still more preferably less than 10% of the composition and most preferably less than 5% of the composition by weight.

The invention also includes the substantially pure said polypeptides when combined with other components ex vivo, said other components not being all of the components found in the cell in which said polypeptides are found.

A further aspect of the invention provides a cell capable of expressing LKB1, STRAD and overexpressed or recombinant MO25. The cell may comprise a recombinant nucleic acid encoding MO25, and/or a recombinant nucleic acid encoding LKB1, and/or a recombinant nucleic acid encoding STRAD. The cell may be capable of overexpressing MO25 from the endogenous sequence encoding MO25, for example using techniques of sequence-specific targeting of transcription activators. The cell may be a prokaryotic or eukaryotic cell. For example it may be a eukaryotic cell, for example an insect, yeast or mammalian cell, for example a human cell. Examples of suitable cells are described, for example, in the Examples.

The recombinant nucleic acid is preferably suitable for expressing the encoded polypeptide. The recombinant nucleic acid may be in the form of an expression vector. Recombinant polynucleotides suitable for expressing a given polypeptide are well known to those skilled in the art, and examples are described in Examples 1 and 2.

A further aspect of the invention provides a cell comprising LKB1, STRAD and overexpressed or recombinant MO25. The cell may comprise recombinant LKB1 and/or recombinant STRAD. The cell may be a cell according to the preceding aspect of the invention. The cell may comprise at least 1.1, 1.2, 1.5, 2, 3, 5, 10 or 20-fold more MO25 (or LKB1, or STRAD, as appropriate) than an equivalent cell which has not been modified in order to overexpress MO25 or to express recombinant MO25.

By “suitable for expressing” is mean that the polynucleotide is a polynucleotide that may be translated to form the polypeptide, for example RNA, or that the polynucleotide (which is preferably DNA) encoding the polypeptide of the invention is inserted into an expression vector, such as a plasmid, in proper orientation and correct reading frame for expression. The polynucleotide may be linked to the appropriate transcriptional and translational regulatory control nucleotide sequences recognised by any desired host; such controls may be incorporated in the expression vector.

Characteristics of vectors suitable for replication in mammalian/eukaryotic cells are well known to those skilled in the art, and examples are given below. It will be appreciated that a vector may be suitable for replication in both prokaryotic and eukaryotic cells.

A variety of methods have been developed to operably link polynucleotides, especially DNA, to vectors for example via complementary cohesive termini. Suitable methods are described in Sambrook et al (1989) Molecular Cloning, A Laboratory Manual, Cold Spring Harbor Laboratory, Cold Spring Harbor, N.Y.

A desirable way to modify the DNA encoding a polypeptide of the invention is to use the polymerase chain reaction as disclosed by Saiki et al (1988) Science 239, 487-491. This method may be used for introducing the DNA into a suitable vector, for example by engineering in suitable restriction sites, or it may be used to modify the DNA in other useful ways as is known in the art.

In this method the DNA to be enzymatically amplified is flanked by two specific primers which themselves become incorporated into the amplified DNA. The said specific primers may contain restriction endonuclease recognition sites which can be used for cloning into expression vectors using methods known in the art.

The DNA (or in the case of retroviral vectors, RNA) is then expressed in a suitable host to produce a polypeptide comprising the compound of the invention. Thus, the DNA encoding the polypeptide constituting the compound of the invention may be used in accordance with known techniques, appropriately modified in view of the teachings contained herein, to construct an expression vector, which is then used to transform an appropriate host cell for the expression and production of the polypeptide of the invention. Such techniques include those disclosed in U.S. Pat. No. 4,440,859 issued 3 Apr. 1984 to Rutter et al, U.S. Pat. No. 4,530,901 issued 23 Jul. 1985 to Weissman, U.S. Pat. No. 4,582,800 issued 15 Apr. 1986 to Crowl, U.S. Pat. No. 4,677,063 issued 30 June 1987 to Mark et al, U.S. Pat. No. 4,678,751 issued 7 Jul. 1987 to Goeddel, U.S. Pat. No. 4,704,362 issued 3 Nov. 1987 to Itakura et al, U.S. Pat. No. 4,710,463 issued 1 Dec. 1987 to Murray, U.S. Pat. No. 4,757,006 issued 12 Jul. 1988 to Toole, Jr. et al, U.S. Pat. No. 4,766,075 issued 23 Aug. 1988 to Goeddel et al and U.S. Pat. No. 4,810,648 issued 7 Mar. 1989 to Stalker, all of which are incorporated herein by reference.

The DNA (or in the case of retroviral vectors, RNA) encoding the polypeptide may be joined to a wide variety of other DNA sequences for introduction into an appropriate host. The companion DNA will depend upon the nature of the host, the manner of the introduction of the DNA into the host, and whether episomal maintenance or integration is desired.

Generally, the DNA is inserted into an expression vector, such as a plasmid, in proper orientation and correct reading frame for expression. If necessary, the DNA may be linked to the appropriate transcriptional and translational regulatory control nucleotide sequences recognised by the desired host, although such controls are generally available in the expression vector. The vector is then introduced into the host through standard techniques. Generally, not all of the hosts will be transformed by the vector. Therefore, it will be necessary to select for transformed host cells. One selection technique involves incorporating into the expression vector a DNA sequence, with any necessary control elements, that codes for a selectable trait in the transformed cell, such as antibiotic resistance. Alternatively, the gene for such selectable trait can be on another vector, which is used to co-transform the desired host cell.

Host cells that have been transformed by the recombinant DNA of the invention are then cultured for a sufficient time and under appropriate conditions known to those skilled in the art in view of the teachings disclosed herein to permit the expression of the polypeptide, which can then be recovered.

Many expression systems are known, including bacteria (for example E. coli and Bacillus subtilis), yeasts (for example Saccharomyces cerevisiae), filamentous fungi (for example Aspergillus), plant cells, animal cells and insect cells.

The vectors include a prokaryotic replicon, such as the ColE1 ori, for propagation in a prokaryote, even if the vector is to be used for expression in other, non-prokaryotic, cell types. The vectors can also include an appropriate promoter such as a prokaryotic promoter capable of directing the expression (transcription and translation) of the genes in a bacterial host cell, such as E. coli, transformed therewith.

A promoter is an expression control element formed by a DNA sequence that permits binding of RNA polymerase and transcription to occur. Promoter sequences compatible with exemplary bacterial hosts are typically provided in plasmid vectors containing convenient restriction sites for insertion of a DNA segment of the present invention.

Typical prokaryotic vector plasmids are pUC18, pUC19, pBR322 and pBR329 available from Biorad Laboratories, (Richmond, Calif., USA) and pTrc99A and pKK223-3 available from Pharmacia, Piscataway, N.J., USA.

A typical mammalian cell vector plasmid is pSVL available from Pharmacia, Piscataway, N.J., USA. This vector uses the SV40 late promoter to drive expression of cloned genes, the highest level of expression being found in T antigen-producing cells, such as COS-1 cells.

An example of an inducible mammalian expression vector is pMSG, also available from Pharmacia. This vector uses the glucocorticoid-inducible promoter of the mouse mammary tumour virus long terminal repeat to drive expression of the cloned gene.

Useful yeast plasmid vectors are pRS403-406 and pRS413-416 and are generally available from Stratagene Cloning Systems, La Jolla, Calif. 92037, USA. Plasmids pRS403, pRS404, pRS405 and pRS406 are Yeast Integrating plasmids (YIps) and incorporate the yeast selectable markers HIS3, TRP1, LEU2 and URA3. Plasmids pRS413-416 are Yeast Centromere plasmids (YCps).

The host cell can be either prokaryotic or eukaryotic. Bacterial cells are preferred prokaryotic host cells and typically are a strain of E. coli such as, for example, the E. coli strains DH5 available from Bethesda Research Laboratories Inc., Bethesda, Md., USA, and RR1 available from the American Type Culture Collection (ATCC) of Rockville, Md., USA (No ATCC 31343). Preferred eukaryotic host cells include yeast, insect and mammalian cells, preferably vertebrate cells such as those from a mouse, rat, monkey or human fibroblastic cell line. Yeast host cells include YPH499, YPH500 and YPH501 which are generally available from Stratagene Cloning Systems, La Jolla, Calif. 92037, USA. Preferred mammalian host cells include human embryonic kidney 293 cells (see Example 1), Chinese hamster ovary (CHO) cells available from the ATCC as CCL61, NIH Swiss mouse embryo cells NIH/3T3 available from the ATCC as CRL 1658, and monkey kidney-derived COS-1 cells available from the ATCC as CRL 1650. Preferred insect cells are Sf9 cells which can be transfected with baculovirus expression vectors.

Transformation of appropriate cell hosts with a DNA construct is accomplished by well known methods that typically depend on the type of vector used. With regard to transformation of prokaryotic host cells, see, for example, Cohen et al (1972) Proc. Natl. Acad. Sci. USA 69, 2110 and Sambrook et al (1989) Molecular Cloning, A Laboratory Manual, Cold Spring Harbor Laboratory, Cold Spring Harbor, N.Y. Transformation of yeast cells is described in Sherman et al (1986) Methods In Yeast Genetics, A Laboratory Manual, Cold Spring Harbor, N.Y. The method of Beggs (1978) Nature 275, 104-109 is also useful. With regard to vertebrate cells, reagents useful in transfecting such cells, for example calcium phosphate and DEAE-dextran or liposome formulations, are available from Stratagene Cloning Systems, or Life Technologies Inc., Gaithersburg, Md. 20877, USA.

Electroporation is also useful for transforming and/or transfecting cells and is well known in the art for transforming yeast cell, bacterial cells, insect cells and vertebrate cells.

For example, many bacterial species may be transformed by the methods described in Luchansky et al (1988) Mol. Microbiol. 2, 637-646 incorporated herein by reference. The greatest number of transformants is consistently recovered following electroporation of the DNA-cell mixture suspended in 2.5× PEB using 6250V per cm at 25:FD.

Methods for transformation of yeast by electroporation are disclosed in Becker & Guarente (1990) Methods Enzymol. 194, 182.

Successfully transformed cells, ie cells that contain a DNA construct of the present invention, can be identified by well known techniques. For example, cells resulting from the introduction of an expression construct of the present invention can be grown to produce the polypeptide of the invention. Cells can be harvested and lysed and their DNA content examined for the presence of the DNA using a method such as that described by Southern (1975) J. Mol. Biol. 98, 503 or Berent et al (1985) Biotech. 3, 208.

In addition to directly assaying for the presence of recombinant DNA, successful transformation can be confirmed by well known immunological methods when the recombinant DNA is capable of directing the expression of the protein. For example, cells successfully transformed with an expression vector produce proteins displaying appropriate antigenicity. Samples of cells suspected of being transformed are harvested and assayed for the protein using suitable antibodies.

Thus, in addition to the transformed host cells themselves, the present invention also contemplates a culture of those cells, preferably a monoclonal (clonally homogeneous) culture, or a culture derived from a monoclonal culture, in a nutrient medium.

A further aspect of the invention method for making a preparation of the invention, comprising the step of purifying the preparation from a cell according to the invention. Methods of cultivating host cells and isolating recombinant proteins are well known in the art. Examples of suitable purification techniques are described in the Examples. For example, one or more component of the preparation may be tagged so as to aid purification using affinity reagents, as will be well known to those skilled in the art and as described in the Examples. Chromatographic techniques may also be used, for example as described in the Examples.

A further aspect of the invention provides a preparation obtained or obtainable by the method of the preceding aspect of the invention. The preparation may comprise, for example, tagged LKB1, STRAD or MO25. The ratios of LKB1:STRAD:MO25 may be 1:1:1, for example as measured using techniques as described in the Examples. It will be appreciated that the ratio determined may vary depending upon the details of the method used in providing the preparation. The preparation may comprise a complex comprising the LKB1, STRAD and MO25, as described in the Examples.

The method of the first aspect of the invention may be performed with LKB1 in the form of a preparation of the second aspect of the invention; or a preparation or complex obtained or obtainable by the method as indicated above; or in a cell of the invention.

The above polypeptides may be made by methods well known in the art and as described below and in Example 1 or 2, for example using molecular biology methods or automated chemical peptide synthesis methods.

It will be appreciated that peptidomimetic compounds may also be useful. Thus, by “polypeptide” or “peptide” we include not only molecules in which amino acid residues are joined by peptide (—CO—NH—) linkages but also molecules in which the peptide bond is reversed. Such retro-inverso peptidomimetics may be made using methods known in the art, for example such as those described in Mézière et al (1997) J. Immunol. 159, 3230-3237, incorporated herein by reference. This approach involves making pseudopeptides containing changes involving the backbone, and not the orientation of side chains. Mézière et al (1997) show that, at least for MHC class II and T helper cell responses, these pseudopeptides are useful. Retro-inverse peptides, which contain NH—CO bonds instead of CO—NH peptide bonds, are much more resistant to proteolysis.

Similarly, the peptide bond may be dispensed with altogether provided that an appropriate linker moiety which retains the spacing between the Cα atoms of the amino acid residues is used; it is particularly preferred if the linker moiety has substantially the same charge distribution and substantially the same planarity as a peptide bond.

It will be appreciated that the peptide may conveniently be blocked at its N- or C-terminus so as to help reduce susceptibility to exoproteolytic digestion.

Thus, it will be appreciated that the STRAD or MO25 polypeptide may be a peptidomimetic compound.

A further aspect of the invention provides a method for identifying a compound for modulating cellular LKB1 activity, the method comprising the steps of (1) determining whether a test compound modulates (for example increases) the LKB1 protein kinase activity of a preparation or complex or cell as defined in relation to any preceding aspect of the invention and (2) selecting a compound which modulates the said LKB1 protein kinase activity. The LKB1 protein kinase activity may b measured using, for example, myelin basic protein as a substrate, or using AMPK or AMPK subfamily member or a fragment thereof (as discussed above and in Example 3) as the substrate. A compound which modulates, for example increases LKB1 protein kinase activity may be useful in medicine, for example for treating cancer.

The compounds identified in the methods may themselves be useful as a drug or they may represent lead compounds for the design and synthesis of more efficacious compounds.

The compound may be a drug-like compound or lead compound for the development of a drug-like compound for each of the above methods of identifying a compound. It will be appreciated that the said methods may be useful as screening assays in the development of pharmaceutical compounds or drugs, as well known to those skilled in the art.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 daltons. A drug-like compound may additionally exhibit features of selective interaction with a particular protein or proteins and be bioavailable and/or able to penetrate cellular membranes, but it will be appreciated that these features are not essential.

The term “lead compound” is similarly well known to those skilled in the art, and may include the meaning that the compound, whilst not itself suitable for use as a drug (for example because it is only weakly potent against its intended target, non-selective in its action, unstable, difficult to synthesise or has poor bioavailability) may provide a starting-point for the design of other compounds that may have more desirable characteristics.

It will be understood that it will be desirable to identify compounds that may modulate the activity of the protein kinase in vivo. Thus it will be understood that reagents and conditions used in the method may be chosen such that the interactions between, for example, the said LKB1 and, for example, the STRAD or MO25 polypeptides, are substantially the same as between the human LKB1 and human STRAD and/or MO25. It will be appreciated that the compound may bind to the LKB1, or may bind to the STRAD or MO25.

The compounds that are tested in the screening methods of the assay or in other assays in which the ability of a compound to modulate the protein kinase activity of a protein kinase, for example LKB1, may be measured, may be compounds that have been selected and/or designed (including modified) using molecular modelling techniques, for example using computer techniques. The selected or designed compound may be synthesised (if not already synthesised) and tested for its effect on the LKLB1, for example its effect on the protein kinase activity. The compound may be tested in a screening method of the invention.

It will be appreciated that screening assays which are capable of high throughput operation will be particularly preferred. Examples may include the cell based assays described and protein-protein binding assays. A further example is an SPA-based (Scintillation Proximity Assay; Amersham International) system as well known to those skilled in the art. Other methods of detecting polypeptide/polypeptide interactions include ultrafiltration with ion spray mass spectroscopy/HPLC methods or other physical and analytical methods. Fluorescence Energy Resonance Transfer (FRET) methods, for example, well known to those skilled in the art, may be used, in which binding of two fluorescent labelled entities may be measured by measuring the interaction of the fluorescent labels when in close proximity to each other

A further aspect of the invention is a compound identified or identifiable by a screening method of the invention.

A still further aspect of the invention is a compound of the invention for use in medicine.

The compound may be administered in any suitable way, usually parenterally, for example intravenously, intraperitoneally, subcutaneous or intramuscular or intravesically, in standard sterile, non-pyrogenic formulations of diluents and carriers. The compound may also be administered topically. The compound may also be administered in a localised manner, for example by injection. The treatment may consist of a single dose or a plurality of doses over a period of time. The compound may be useful as an anticancer agent or for the treatment of, for example, diabetes or obesity.

Whilst it is possible for a compound of the invention to be administered alone, it is preferable to present it as a pharmaceutical formulation, together with one or more acceptable carriers. The carrier(s) must be “acceptable” in the sense of being compatible with the compound of the invention and not deleterious to the recipients thereof. Typically, the carriers will be water or saline which will be sterile and pyrogen free.

Thus, the invention also provides pharmaceutical compositions comprising the compound identified or identifiable by the screening methods of the invention and a pharmaceutically acceptable carrier.

A further aspect of the invention provides a kit of parts comprising LKB1 or a recombinant polynucleotide encoding LKB1, STRAD or a recombinant polynucleotide encoding STRAD, and MO25 or a recombinant polynucleotide encoding MO25. A further aspect of the invention provides a kit of parts comprising (1) AMPK or AMPK subfamily member, or recombinant polynucleotide encoding AMPK or AMPK subfamily member (including a fragment thereof as discussed above or in Example 3) and (2) a kit of parts as defined in relation to the preceding aspect of the invention, or a preparation or complex or cell of the invention. Such kits may be useful in forming a preparation or complex which may be useful in, for example a screening method of the first aspect of the invention. The recombinant polynucleotide(s) may be in an expression vector (for example as discussed above) or (less desirably) useful for in vitro expression.

A further aspect of the invention provides a method for overexpressing LKB1 comprising the steps of (1) selecting a cell type in which to overexpress LKB1, comprising the step of determining whether the cell type is one that expresses STRAD and/or MO25, and (2) overexpressing LKB1 in the selected cell type. Methods for determining whether the cell type expresses STRAD and/or MO25 will be well known to those skilled in the art, and examples of such methods are described in the Examples. Alternatively, the cell type may be one that is already known to express STRAD and/or MO25. Examples of such cells include 293, HeLa, G361 and Rat2 cells. STRADα and MO25α are expressed at good levels in most cells analysed.

A further aspect of the invention provides a method for preparing LKB1 comprising the steps of (1) overexpressing LKB1 in a cell using a method according to the preceding aspect of the invention and (2) preparing LKB1 from the cell. By this is included preparing LKB1 in a preparation or complex with STRAD and MO25. Suitable methods for preparing LKB1 are described in the Examples.

Overexpression of LKB1 may lead to growth arrest, for example in eukaryotic cells. It may therefore be necessary to express LKB1 using a transient expression system, for example as described in the Examples, or using a regulated (inducible) expression system, as well known to those skilled in the art.

A further aspect of the invention provides a method for identifying a putative binding partner for MO25 comprising the steps of (1) providing an amino acid sequence of at least the C-terminal three amino acids of a test putative binding partner (2) selecting a putative binding partner having the C-terminal amino acid sequence Trp-Glu/Asp-Phe. The method may further comprise the step of determining that the selected putative binding partner binds to MO25, for example using methods for assessing molecular interactions as indicated above. The motif Trp-Glu/Asp-Phe is considered to be involved in the interaction between MO25 and STRAD.

A further aspect of the invention provides a method for identifying a genetic difference associated with PJS (Peutz-Jeghers Syndrome) comprising the steps of (1) investigating the sequence of a gene encoding a STRAD or MO25 isoform in at least one patient having PJS (2) identifying any difference between the said patient sequence and equivalent sequence from an individual without PJS. The association of LKB1 with STRAD and MO25 and the association of LKB1 deficiency with PJS suggests that defects in MO25 or STRAD could also be involved in PJS. Techniques for identifying mutations in candidate genes, and for investigating whether a mutation is linked with disease or is not so linked will be well known to those skilled in the art.

A further aspect of the invention provides a method for determining whether an individual is susceptible to PJS comprising the steps of determining whether the test individual has a genetic difference identified as associated with PJS by a method according to the preceding aspect of the invention. It may also be useful to determine whether an individual who has PJS has a genetic difference identified as associated with PJS by a method according to the preceding aspect of the invention, in order to determine what treatment may be suitable. For example, a patient who is found to have a defect in STRAD or MO25 may be treated using STRAD or MO25 or a genetic construct which allows STRAD or MO25 to be expressed in cells where it is needed.

Thus, the invention provides STRAD or MO25 or a recombinant polynucleotide encoding same for use in medicine. A further aspect of the invention provides the use of STRAD or MO25 or a recombinant polynucleotide encoding same in the manufacture of a medicament for treating cancer, PJS, diabetes or obesity. The recombinant polynucleotide may be in the form of a gene therapy vector, for example a viral gene therapy vector, as well known to those skilled in the alt.

A further aspect of the invention method for identifying a compound which activates AMPK or AMPK subfamily member by a similar mechanism to metformin or phenformin or AICA riboside in which the effect of a test compound on the activation of AMPK or AMPK subfamily member by a preparation or complex or cell of the invention is compared with the effect of metformin or phenformin or AICA riboside on the activation of AMPK or AMPK subfamily member and a compound with a similar effect is selected. Such a method may be useful in determining whether a compound is more potent than metformin or phenformin or AICA riboside.

A further aspect of the invention provides the use of an AMPK subfamily member (other than AMPK) or polynucleotide encoding an AMPK subfamily member (other than AMPK) in the manufacture of a medicament for treating diabetes or obesity.

A further aspect of the invention provides a peptide substrate for LKB1 comprising the amino acid sequence LSNLYHQGKFLQTFCGSPLY or FGNFYKSGEPLSTWCGSPPY or LSNMMSDGEFLRTSCGSPNY or MASLQVGDSLLETSCGSPHY or FSNEFTVGGKLDTFCGSPPY or AKPKGNKDYHLQTCCGSLAY; or a said sequence with from one to four substitutions therein at any position other than the underlined residue and/or a conservative substitution at the underlined residue; or at least ten contiguous residues of a said sequence encompassing the underlined residue. The peptide substrate typically has less than about 30 or 25 residues, for example, 22, 21, 20, 19, 18, 17, 16, 15, 14, 13, 12, 11 or 10 residues. Within this range, longer peptides may have better affinity for the LKB1 complex, whilst shorter peptides may have better solubility in aqueous liquids.

In particular, the peptide substrate for LKB1 may consist of the amino acid sequence LSNLYHQGKFLQTFCGSPLY or LSNLYHQGKFLQTFCGSPLYRRR or SNLYHQGKFLQTFCGSPLY or SNLYHQGKFLQTFCGSPLYRRR or LSNLYHQGKFLQTFCGSPLY or LSNLYHQGKFLQTFCGSPLYRRR or FGNFYKSGEPLSTWCGSPPY or FGNFYKSGEPLSTWCGSPPYRRR or LSNMMSDGEFLRTSCGSPNY or LSNMMSDGEFLRTSCGSPNYRRR or MASLQVGDSLLETSCGSPHY or MASLQVGDSLLETSCGSPHYRRR or FSNEFTVGGKLDTFCGSPPY or FSNEFTVGGKLDTFCGSPPYRRR or AKPKGNKDYHLQTCCGSLAY or AKPKGNKDYHLQTCCGSLAYRRR. The tripeptide RRR may be useful in anchoring the peptide on p81 membrane and may be replaced by a different anchoring moiety, for example if a different support is used.

A further aspect of the invention provides an antibody reactive with a peptide antigen having the amino acid sequence MVAGLTLGKGPESPDGDVS (residues 1-20 of human BRSK1), LSWGAGLKGQKVATSYESSL (residues 655-674 of human BRSK2), MEGAAAPVAGDRPDLGLGAPG (residues 1-21 of human NUAK1), TDCQEVTATYRQALRVCSKLT (residues 653-673 of human NUAK2), MVMADGPRHLQRGPVRVGFYD (residues 1-21 of human QIK), MVIMSEFSADPAGQGQGQQK (residues 1-20 of human SIK), GDCEMEDLMPCSLGTFVLVQ (residues 765-784 of human SIK), TDILLSYKHPEVSFSMEQAGV (residues 1349-1369 of human QSK), SGTSIAFKNIASKIANELKL (residues 776-795 of human MARK1), MSSRTVLAPGNDRNSDTHGT (residues 1-20 of human MARK4), MKDYDELLKYYELHETIGTG (residues 1-20 of human MELK), CTSPPDSFLDDHHLTR (residues 344-358 of rat AMPKα1), or CDPMKRATIKDIRE (residues 252 to 264 of rat AMPKα1). Such an antibody may be raised using the given peptide as immunogen, as well known to those skilled in the art. Alternative methods of obtaining such an antibody will also be well known to those skilled in the art, for example using phage display techniques.

By the term “antibody” is included synthetic antibodies and fragments and variants (for example as discussed above) of whole antibodies which retain the antigen binding site. The antibody may be a monoclonal antibody, but may also be a polyclonal antibody preparation, a part or parts thereof (for example an F_{ab} fragment or F(ab′)₂) or a synthetic antibody or part thereof. Fab, Fv, ScFv and dAb antibody fragments can all be expressed in and secreted from E. coli, thus allowing the facile production of large amounts of the said fragments. By “ScFv molecules” is meant molecules wherein the V_{H} and V_{L} partner domains are linked via a flexible oligopeptide. IgG class antibodies are preferred.

Suitable monoclonal antibodies to selected antigens may be prepared by known techniques, for example those disclosed in “Monoclonal Antibodies: A manual of techniques”, H. Zola (CRC Press, 1988) and in “Monoclonal Hybridoma Antibodies: techniques and Applications”, J G R Hurrell (CRC Press, 1982), modified as indicated above. Bispecific antibodies may be prepared by cell fusion, by reassociation of monovalent fragments or by chemical cross-linking of whole antibodies. Methods for preparing bispecific antibodies are disclosed in Corvalen et al, (1987) Cancer Immunol. Immunother. 24, 127-132 and 133-137 and 138-143.

A general review of the techniques involved in the synthesis of antibody fragments which retain their specific binding sites is to be found in Winter & Milstein (1991) Nature 349, 293-299.

A further aspect of the invention provides a mutated AMPK subfamily member wherein the T-loop threonine residue corresponding to Thr172 of AMPKα1 is mutated to an alanine residue or to an aspartate or glutamate residue. A further aspect of the invention provides a polynucleotide encoding a mutated AMPK subfamily member wherein the T-loop threonine residue corresponding to Thr172 of AMPKα1 is mutated to an alanine residue or to an aspartate or glutamate residue. References for the sequences of AMPK subfamily members are indicated above, as is teaching relevant to these aspects of the invention relating to recombinant polynucleotides and polypeptides, for example methods for performing mutagenesis and for expressing recombinant polypeptides. Appropriate techniques will also be known to those skilled in the art. The mutated AMPK subfamily member may be, for example, BRSK1, BRSK2, NUAK1, NUAK2, QIK, SIK or MELK.

Published documents referred to herein are hereby incorporated by reference.

The invention is now described further by reference to the following, non-limiting, Figures and Examples.

### FIGURE LEGENDS

FIG. 1. Association of MO25α with LKB1. (A) Cell lysates derived from control parental HeLa cells or HeLa cells stably expressing N-terminal Flag epitope tagged wild type (WT) LKB1 or kinase dead (KD) LKB1 were passed through an anti-Flag M2-affinity agarose column, LKB1 eluted with the Flag peptide and the samples concentrated as described in Materials and Methods. The samples were electrophoresed on a polyacrylamide gel and the protein bands visualised following colloidal blue staining. Protein bands unique to the WT and KD LKB1 preparations are indicated. (B) The colloidal blue-stained bands labelled as indicated in (A) were excised from the gel, the proteins digested in gel with trypsin, and their identities were determined by tryptic peptide mass-spectral fingerprint. The identity of STRADα and MO25α were confirmed by LC-MS/MS sequence analysis on a Q-TOF2 mass spectrometer. The number of tryptic peptides, percentage of sequence coverage and NCBI gi numbers for each protein identified are indicated. (C) The samples purified in (A) were immunoblotted with the indicated antibodies. Identical results were obtained following 2 independent purifications of LKB1 from HeLa cells.

FIG. 2. Amino acid sequence and tissue distribution patterns of MO25α and MO25β forms. (A) Amino acid sequence alignment of the human MO25α (NCBI accession number NP_{—}057373) and MO25 (NCBI accession number Q9H9S4) isoforms as well as C. elegans MO25α (NCBI accession number CAB16486) and MO25β (NCBI accession number NP_{—}508691) and Drosophila MO25 (NCBI accession number P91891) putative homologues. Conserved residues are boxed in black, and homologous residues are shaded in grey. Sequence alignments were performed using the CLUSTALW and BOXSHADE programmes at http://www.ch.embnet.org/ using standard parameters. (B) A ³²P-labelled fragment of the MO25α cDNA was used to probe a Northern blot containing polyadenylated RNA isolated from the indicated human tissues. The membrane was autoradiographed, and the MO25α probe was observed to hybridise to a 4.2-kb message, identical to the size predicted for the MO25α message from database analysis. As a loading control, the Northern Blot was hybridised with a β-actin probe. (C &D) The indicated mouse tissue (C) or cell (D) extracts containing 20 μg of total cell protein were immunoblotted with the anti-MO25α and anti-MO25β antibodies.

FIG. 3. Endogenous LKB1 is associated with MO25α. (A) LKB1 was immunoprecipitated from 2 mg of the indicated lysates using 10 μg of anti-LKB1 antibody covalently coupled to protein G-Sepharose, and the immunoprecipitates were immunoblotted with the indicated antibodies. As a control, immunoprecipitations were also performed in parallel experiments with preimmune IgG antibodies covalently coupled to protein G-Sepharose. In each gel, 20 μg of total cell lysate was also immunoblotted in parallel. (B) MO25α was immunoprecipitated as above except that 15 μg of the MO25α antibody was employed. The results shown are from a single experiment that was repeated 3 times with similar results.

FIG. 4. MO25α is associated with LKB1 through STRAD (A) 293 cells were transfected with plasmids encoding for the expression of the indicated GST fusion proteins together with Myc-MO25α. 36 h post transfection, the GST tagged proteins were affinity purified from the cell lysates using glutathione-Sepharose as described in Materials and Methods. Similar amounts of the purified GST-fusion proteins were subjected to SDS-polyacrylamide gel electrophoresis and immunoblotted with anti-Myc antibody to detect co-purified Myc-MO25α, or with anti-GST antibody to ensure that comparable amounts of the GST tagged proteins were present in each lane (upper and middle panels). 5 μg of total cell lysates prior to affinity purification were also subjected to immunoblotting with anti-Myc antibody to ensure that Myc-MO25α was expressed at similar levels in each co-transfection (lower panel). (B) N-terminal GST tagged LKB1 was expressed in 293 cells in the presence or absence of Flag-STRADα and purified as above. The purified proteins were subjected to SDS-polyacrylamide gel electrophoresis and visualised by Colloidal blue staining (upper panel). The faint protein band indicated, corresponding to an endogenous protein of 40-kDa (marked *), was identified by tryptic peptide mass-spectral fingerprint as endogenously expressed MO25α (with 38% sequence coverage). The purified proteins were also immunoblotted with the indicated antibodies (lower panels). (C) As above except that GST-LKB1 was expressed in 293 cells together with the indicated isoforms of Flag-STRAD and Myc-MO25 and the proteins visualised by colloidal blue staining were also immunoblotted with the indicated antibodies. For all panels, similar results were obtained in at least 3 separate experiments.

FIG. 5. MO25α and STRADα anchor LKB1 in the cytoplasm. HeLa cells were transfected with the indicated constructs encoding for the expression of Myc-MO25α, Flag-STRADα and GFP-LKB1. 24 h post transfections the cells were fixed in 4% (by vol) paraformaldehyde and immunostained with anti-MO25α antibody to detect MO25α (TR anti-sheep secondary antibody, red channel) and anti-Flag to detect STRADα (Cy5 anti-mouse secondary antibody, blue channel). GFP-LKB1 localisation was visualised directly through the GFP fluorescence (green channel). The cells were imaged using a Zeiss LSM 510 META confocal microscope. The cells shown are representative images obtained in 3 separate experiments. The scale bars correspond to 10 μm.

FIG. 6. MO25α recognises the C-terminal 3 residues of STRADα. (A) N-terminal GST tagged wild type STRADα or the indicated mutants of STRADα were expressed in 293 cells together with Myc-MO25α, and 36 h post transfection the STRADα proteins were affinity purified from the cell lysates using glutathione-Sepharose. Similar amounts of the purified proteins were subjected to SDS-polyacrylamide gel electrophoresis and immunoblotting with anti-Myc antibody to detect co-purified Myc-MO25α, or with anti-GST antibody to ensure that comparable amounts of the GST tagged proteins were present in each lane (upper and middle panels). 5 μg of the total cell lysates prior to affinity purification were also subjected to immunoblotting with anti-Myc antibody to ensure that Myc-MO25α was expressed at similar levels in each condition (lower panel). (B) 0.5 mg of the indicated cell lysates was incubated with 5 μg of an N-terminal biotinylated peptide encompassing either the C-terminal 12 residues STRADα conjugated to streptavidin-Sepharose (NLEELEVDDWEF, termed STRADα-C12) or mutants of this peptide in which the indicated residues were individually mutated to Ala. Following isolation and washing of the beads, the samples were subjected to SDS-polyacrylamide gel electrophoresis and immunoblotted with an anti-MO25α antibody. (C) Binding of bacterially expressed MO25α to the indicated peptides was analysed by surface plasmon resonance BiaCore analysis as described in Materials and Methods. Binding was analysed over a range of MO25α concentrations (6.25-3200 nM) and the response level at the steady-state binding was plotted versus the log of the MO25α concentration. The estimated K_{d} for the STRADα-C12 peptide was obtained by fitting the data to the formula [m1×m0/(m0+m2)] using Kaleidagraph software and the K_{d} was calculated to be 850 nM. WEF-C12 corresponds to NLEELEVDDWEF, WEA-C12 corresponds to NLEELEVDDWEA, AEF-C12 corresponds to NLEELEVDDAEF, WAF-C12 corresponds to NLEELEVDDWAF, WEF-C6 corresponds to VDDWEF.

FIG. 7. Binding of LKB1 to STRADα creates novel binding site(s) for MO25α. The indicated forms of GST-STRADα were co-expressed in 293 cells together with Myc-MO25α and/or wild type Flag-LKB1 or the isolated LKB1 catalytic domain (LKB1[44-343]. 36 h post-transfection, the GST-STRADα proteins were affinity purified, subjected to SDS-polyacrylamide gel electrophoresis and immunoblotting with anti-Myc antibody to detect Myc-MO25α, anti-Flag antibody to detect co-purified Flag-LKB1 and Flag-LKB1[44-343], or with anti-GST antibody to detect STRADα forms (upper panels). 5 μg of total cell lysates prior to affinity purification were also subjected to immunoblotting with anti-Myc and anti-Flag antibodies to ensure that MO25α and LKB1 were expressed at similar levels in each co-transfection (lower panels). Similar results were obtained in 3 separate experiments.

FIG. 8. MO25 isoforms stabilise the LKB10STRADα complex. (A) 293 cells were transfected with 3 μg of the DNA construct encoding for expression of GST-LKB1, in the presence or absence of 3 μg of Flag-STRADα construct and in the absence or presence of indicated amounts of Myc-MO25α construct. 36 h post-transfection, GST-LKB1 was affinity purified from the cell lysates and immunoblotted with appropriate epitope antibodies to detect LKB1, STRADα and MO25α. 5 μg of total cell lysates prior to affinity purification were also immunoblotted with the indicated antibodies (lower panels). (B) is performed as above except that the MO25β construct was employed instead of the MO25α construct. Similar results were obtained in 3 separate experiments.

FIG. 9. Activation of LKB1 by association with STRAD and MO25 Isoforms. 293 cells were transfected with constructs encoding GST-LKB1 in the presence or absence of constructs encoding the indicated isoforms of STRAD and MO25. 36 h post-transfection, GST-LKB1 was affinity purified and assayed for autophosphorylation and transphosphorylation of MBP as described in the materials and methods. The reactions were electrophoresed and the gel autoradiographed (top panel). Phosphorylation of MBP by LKB1 at Thr65 was also monitored by immunoblotting with a phosphospecific antibody (T65-P), which recognises MBP phosphorylated by LKB1 at this residue. Each reaction was also immunoblotted with the appropriate epitope tag antibodies to monitor levels of LKB1, STRAD and MO25 isoforms. Similar results were obtained in 3 separate experiments.

FIG. 10. Amino acid sequence alignment of STRAD isoforms with closest STE20-kinase relatives. (A) Amino acid sequence alignment of the human STRADα (NCBI accession number AAG48269) and STRADβ (NCBI accession number AAM19143) with human SPAK (NCBI accession number AAC72238) and human OSR1 (NCBI accession number NP_{—}005100). Conserved residues are boxed in black, and homologous residues are shaded in grey. The catalytic Asp (subdomain VI) and Asp-Phe-Gly (subdomain VII) present in the active SPAK and OSR1 protein kinases, but lacking in the STRADα and STRADβ pseudokinases are boxed and marked with an asterisk. The C-terminal Trp-Glu-Phe motif present in STRADα and STRADβ corresponding to a Phe-Glu-Phe sequence in SPAK and OSR1 are boxed and marked with triangles and the sites of LKB1 phosphorylation in STRADα (Thr329 and Thr419) (Baas et al., 2003) are marked with arrows. Sequence alignments were performed using the CLUSTALW and BOXSHADE programmes at http://www.ch.embnet.orz/ using standard parameters.

FIG. 11. MO25 Isoforms Increase expression of STRADβ and formation of the LKB1-STRADβ complex. (A) 293 cells were transfected with 3 μg of the DNA construct encoding for expression of GST-LKB1, in the presence or absence of 3 μg of Flag-STRADβ construct and in the absence or presence of indicated amounts of Myc-MO25α construct. 36 h post-transfection, GST-LKB1 was affinity purified from the cell lysates and immunoblotted with appropriate epitope antibodies to detect LKB1, STRADβ and MO25α. 5 μg of total cell lysates prior to affinity purification were also immunoblotted with the indicated antibodies (lower panels). (B) is performed as above except that the MO25β construct was employed instead of the MO25α construct. Similar results were obtained in 3 separate experiments and also when STRADβ was expressed as a GST-fusion protein in the pEBG-2T vector instead of the pCMV5 vector (data not shown).

FIG. 12: Alignment of the amino acid sequences of Tos3, Pak1 and Elm1 from yeast and LKB1 and CaMKKβ (β3 variant) from humans. The alignment was created using PILEUP from the GCH suite [49] and the consensus sequence determined and colouring added using BOXSHADE v3.2.1 [50]. Residues that are identical in at least half of the sequences are coloured grey, with conservative changes also in grey. The consensus sequence is represented by upper case letter if the residue at a particular position is identical in all sequences, and by a lower case letter if at least half are conserved. Only the conserved central regions containing the kinase domains (residues 50-310 of LKB1 approx.) are shown.

FIG. 13: Two AMPKKs can be resolved from rat liver extracts but both are complexes between LKB1, STRADα and MO25α. (A) Separation of two activities that activate GST-AMPKα1-catalytic domain by Q-Sepharose chromatography. The graph shows AMPKK activity in fractions (open circles, absorbance at 280 nm (continuous line) and conductivity in the eluate (dashed line). The “x” axis represents the fraction number (4.5 ml fractions). B, C, D: probing of blots of column fractions after SDS-PAGE (1 μl per lane) using anti-LKB1 (B), anti-STRADα (C) and anti-MO25α (D). E, F, G: Fractions 26-30 were concentrated from 4.5 ml to 250 μl using Amicon Ultra-4 30,000 MWCO centrifugal concentrators, and 2 μl was loaded per lane. Following SDS-PAGE the gels were transferred to nitrocellulose and the membranes probed with anti-LKB1 (E), anti-STRADα (F) and anti-MO25α (G).

FIG. 14: AMPKK activity, and the LKB1, STRADα and MO25α polypeptides, can be immunoprecipitated from rat liver AMPKK1 and AMPKK2 using anti-LKB1 antibody. (A) Depletion of AMPKK activity from supernatant. Sheep anti-human LKB1 or pre-immune control immunoglobulin (IgG) was pre-bound to Protein G-Sepharose beads and crosslinked with DMP as described previously [51], except that a final wash of the beads with 100 mM glycine, pH 2.5, was performed. Bead-bound antibodies (40 μl) were incubated with the peak fraction of AMPKK1 (0.04 Units), AMPKK2 (0.03 Units) or recombinant GST-LKB1:STRADα:MO25α complex (0.06 Units) for 20 minutes and the beads removed in a microcentrifuge (14,000×g, 2 min). AMPKK activity was determined in the supernatants and is expressed as a percentage of the value obtained using the control IgG. (B) The pellets from the experiment in (A) were resuspended in the original volume and samples of the supernatants and pellets analysed by Western blotting with anti-LKB1 antibody. The recombinant LKB1 migrates at a higher molecular mass because of the OST tag. (C) As A, except that the amount of AMPKK1, AMPKK2 and recombinant GST-LKB1-STRADα-MO25α complex were increased to 0.44, 0.70 and 1.4 Units respectively, and the activities were determined in the resuspended pellets. In this experiment the amount of antibody was limiting so only a portion of the activity was precipitated. (D) The pellets from the experiment in (C) were resuspended and samples analysed by Western blotting with anti-LKB1, anti-STRADα and anti-MO25α antibodies.

FIG. 15: Recombinant LKB1-STRAD-MO25 complexes can efficiently activate AMPK via phosphorylatation at Thr-172. (1): the indicated combinations of GST-tagged wild type LKB1 (WT, lanes 1-9), or kinase-dead (D194A, KD, lanes 10-13) LKB1 mutant, or GST-alone (lane 14), FLAG-tagged STRADα or STRADβ, and myc-tagged MO25α or MO25β were co-expressed in HEK-293T cells, purified on glutathione-Sepharose and tested for their ability to activate GST-AMKα1 catalytic domain (top panel). Samples from each incubation were also analysed by Western blotting and probed using the indicated antibodies (from top to bottom(: anti-pT172, anti-AMPKα1 catalytic domain (GST-α1), anti-GST to detect GST-LKB1, anti-FLAG to detect STRADα and STRADβ, and anti-myc to detect MO25α and MO25β. All proteins migrated with the expected mobility, taking into account the epitope tags. The bottom three blots were conducted on blank reactions lacking GST-AMPKα1 catalytic domain, as the latter appeared to cause some interference with detection. (B): recombinant GST-LKB1:STRADα:MO25α complex was used to phosphorylate wild type GST-α1 catalytic domain (GST-α1-WT) or a T172A mutant (GST-α1-T172A) using [γ-32]ATP as described under Methods. The reaction was analysed by SDS-PAGE and autoradiography. Arrows show the migration of GST-LKB1 (which autophosphorylates) and GST-α1 catalytic domain.

FIG. 16: Activation and phosphorylation of heterotrimeric AMPK complexes by AMPKK1, AMPKK2 and recombinant GST-LKB1:STRADα:MO25α complexes. combinant α1β1γ1 or α2β1γ1 complexes where incubated for 10 min with MgATP and AMPKK1 (2.8 or 1.4 units/ml), AMPKK2 (10.3 or 5.15 units/ml) or GST-LKB1:STRADα:MO25α (14.3 or 7.2 units/ml), and AMPK activity determined. Twice as much upstream kinase was added when the α1β1γ1 complex was the substrate, as pilot experiments showed that it was activated less readily. The images are of Western blots probed with phosphospecific anti-pT172 antibody (upper panel), and with a mixture of anti-α1 and -α2 antibodies to demonstrate uniform lodaing of the α1β1γ1 or α2β1γ1 complexes (note that the anti-α2 antibody always gives a stronger signal than anti-α1 even for equal protein loadings).

FIG. 17: Endogenous AMPKK activity can be immunoprecipitated from 293 cells using anti-LKB1 antibody, but activity can only be immunoprecipitated from HeLa cells if they stably express LKB1 (but not a catalytically-inactive mutant) from an inducible promoter. LKB1 was immunoprecipitated from 0.5 mg of cell extract derived from untransfected HEK-293T cells (lanes 1, 2), untransfected HeLa cells (lanes 3, 4), or HeLa cells stably expressing wild type (WT) LKB1 (lanes 5, 6) or a kinase-dead (KD) LKB1 (D194A) mutant (lanes 7, 8) from an inducible promoter. Immunoprecipitation was with anti-LKB1 (lanes 1, 3, 5, 7) or a pre-immune control immuoglobulin (IgG, lanes 2, 4, 6, 8). Samples of each immunoprecipitate were used to assay activation of GST-AMPKα1-catalytic domain, to analyze phosphorylation of GST-AMPKα1-catalytic domain on Thr-172 (middle panel), and to determine recovery of LKB1 (bottom panel). Samples of each immunoprecipitate were also immunoblotted for the presence of endogenous STRADα and MO25α with appropriate antibodies. Also shown in the top panel is the basal activity obtained when the GST-AMPKα1-catalytic domain was incubated with MgATP on its own (no addition).

FIG. 18: Restoration of the ability of AMPK to be activated by AICA riboside and phenformin in HeLa cells by expression of LKB1. Control HeLa cells (lanes 1, 2, 3), HeLa cells expressing wild type (WT) LKB1 (lanes 4, 5, 6) and kinase dead (D194A, KD) mutant LKB1 (lanes 7, 8, 9) were either left unstimulated or treated with 2 mM AICA riboside or 10 mM phenformin for 60 min and lysed. (A) Endogenous AMPK was immunoprecipitated from the cell extracts and assayed. (B) The cell lysates was immunoblotted with antibodies recognising AMPKα1 phosphorylated at Thr172 or total AMPKα1; the results were analysed using the LI-COR IR Odyssesy™ imager as described under Materials and Methods, and are expressed as a ratio of the two signals. (C) The cell lysates was analysed by Western blotting and the membranes probed with antibodies recognising ACC phosphorylated at Ser-79, or streptavidin to determine total AMPKα1. The results were analysed using the LI-COR IR imager as for (B).

FIG. 19: Sequence alignment of the activation loop sequences of AGC sub-family kinases (PKAα and PKCα, which are not thought to be activated by LKB1) with the T-loop residues of the AMPK/SNF1 subfamily of protein kinases. The methods and representation are as described in FIG. 1. The threonine residues corresponding to Thr-172 of AMPK (“P”) and other residues mentioned in the text are indicated with downward arrows. All sequences are from humans, except AtSnRK1-α1, AtSnRK1-α2 and ScSnf1, which are the Arabidopsis thaliana (AtSnRK1, SNF1-related kinase-1) and Saccharomyces cerevisiae (ScSnf1) homologues of AMPK. Although the human, Arabidopsis and S. cerevisiae homologues, as well as the PKAA and PKCA kinases, are known to activated by phosphorylation of the threonine residue equivalent to Thr-172, it has yet to be established whether the other AMPK subfamily members (NuaK1, NuaK2, BrsK1, BrsK2, SIK, QIK, QSK, MELK) are activated by phosphorylation of this residue, and whether LKB1-STRAD-MO25 complexes can mediate this phosphorylation.

FIG. 20: Kₘ and Vₘₐₓ for a peptide substrate (termed AMPKtide). Assay conditions are described in Example 3.

FIG. 21. Activation of AMPK-related kinases by LKB1. (A) Dendrogram and T-loop sequences of AMPK subfamily of protein kinases (Manning et al., 2002). The identical residues are shaded black and the conserved residues in grey. The T-loop Thr and Ser are indicated with an asterisks. (B) The indicated AMPK-related kinases were incubated with wild type LKB1:STRAD:MO25 (open squares) or catalytically inactive LKB1[D194A]:STRAD:MO25 (open circles) complexes in the presence of Mg²⁺ and ATP. At the indicated times, the activity of the AMPK-related kinases was assayed with the AMARA peptide and results are expressed as specific activity. Results shown are means ± SD of assays carried out in triplicate and representative of two independent experiments. The error bars are only shown when larger than the size of the open squares.

FIG. 22. Efficient activation of AMPK-related kinases by LKB1 requires STRAD and MO25 subunits. The indicated combinations of GST-tagged wild type LKB1 (WT, lanes 1-9), or catalytically inactive (D194A, lanes 10-13) LKB1, or OST alone (lane 14), FLAG-tagged STRADα or STRADβ, and myc-tagged MO25α or MO25β were co-expressed in HEK-293T cells and purified on glutathione-Sepharose. The complexes were tested for their ability to activate the catalytic domain of AMPKα1 or the indicated AMPK-related kinases. The results are expressed as specific activity employing the AMARA peptide as substrates. Results shown are means ± SD of assays carried out in triplicate and representative of two independent experiments. Samples from each incubation were also analysed by Western blotting and probed using the indicated antibodies (from top to bottom): anti-GST to detect LKB1; anti-FLAG to detect STRADα and STRADβ; and anti-myc to detect MO25α and MO25β. All proteins migrated with the expected mobility, taking into account the epitope tags.

FIG. 23. The T-loop Thr is the major site of LKB1 phosphorylation on the AMPK-related kinases. Wild type (WT) and T-loop Thr to Ala (Thr→Ala) mutants of the indicated GST-AMPK-related kinases, were incubated with LKB1:STRAD:MO25 complex in the presence of Mg²⁺ and [γ³²P]ATP. Phosphorylation of protein substrates was determined by electrophoresis on a polyacrylamide gel and subsequent autoradiography of the Coomassie Blue-stained bands corresponding to each substrate. An aliquot of each incubation was also analysed by Western blotting probing with an anti-HA antibody to ensure equal loading of wild type and mutant AMPK-related kinases (which all possess a HA epitope tag). All proteins migrated with the expected mobility, taking into account the epitope tags. Similar results were obtained in 3 separate experiments.

FIG. 24. Effect of mutation of the Thr In the T-loop on activation of AMPK-related kinases by LKB1. The indicated wild type (WT) AMPK-related kinases or mutants of these enzymes in which the T-loop Thr was changed to either Ala (T/A) or Glu (T/E), were incubated in the absence (−) or presence (+) of wild type LKB1:STRAD:MO25 in the presence of Mg²⁺ and ATP. After 30 min, the AMPK-related kinases were assayed with the AMARA peptide and results are expressed as specific activity. Results shown are means ± SD of assays carried out in triplicate and representative of two independent experiments. An aliquot of each incubation was also analysed by Western blotting probing with an anti-HA antibody to ensure equal loading of wild type and mutant AMPK-related kinases (which all possess a HA tag).

FIG. 25. Analysis of phosphorylation and activation of MARK kinases. (A) Catalytically inactive MARK3[D196A) (KI), that can not autophosphorylate and the indicated mutants, were incubated with LKB1 complex for 30 min with Mg²⁺-[γ³²P]ATP and separated by electrophoresis on a polyacrylamide gel which was then autoradiographed. The ³²P-labelled MARK3 proteins were digested with trypsin and the resulting ³²P-labelled peptides were chromatographed on a C₁₈ column. Fractions containing the ³²P-labelled T-loop tryptic peptide (Peptides P_{A} and P_{B}) are shown. (B) Peptides P_{A} and P_{B} were subjected to solid phase sequencing and ³²P-radioactivity was measured after each cycle of Edman degradation. In combination with MALDI TOF-TOF mass spectrometry, this enabled the identification of the sites phosphorylated in each peptide. Peptides P_{A} comprises the MARK3 T-loop peptide phosphorylated at Thr211 and Ser215 and Peptides P_{B} the MARK3 T-loop peptide phosphorylated at Thr211. (C to F) The indicated wild type (WT) or mutant forms of MARK kinases in which the the T-loop Thr or Ser was mutated to either Ala (T/A, S/A) or Glu to (T/E, S/E), were incubated in the absence (−) or presence (+) of wild type LKB1:STRAD:MO25 in the presence of Mg²⁺ and ATP. After 30 min, the MARK kinases were assayed using the AMARA peptide, and results are expressed as specific activity. An aliquot of each incubation was also analysed by Western blotting probing with an anti-HA antibody to ensure equal loading of wild type and mutant MARK kinases (which all possessed an HA epitope tag). Results shown are average ± SD of a triplicate assay and results are representative of at least two independent experiments. (G) MARK4 was phosphorylated with LKB1 complex as in A and the major ³²P-labelled peptide was analysed by solid phase Edman sequencing as in (B). In combination with MALDI TOF-TOF mass spectrometry (FIG. 30C), this peptide was shown to comprise the T-loop of MARK4 phosphorylated at only the Thr residue.

FIG. 26. Identification of peptide substrates for LKB1. (A) Kinetic analysis of the phosphorylation of the indicated T-loop by the LKB1:STRAD:MO25 complex was performed. The T-loop Thr residue in each peptide is underlined and in boldface type and 3 Arg residues were added to the C-terminus of each T-loop peptide to enable their capture on phosphocellulose p81 paper. Kₘ and Vₘₐₓ values were determined from nonlinear regression. (B) An aliquot of each peptide phosphorylated by LKB1 complex was subjected to solid phase Edman sequencing and ³²P-radioactivity was measured after each cycle of Edman degradation. A small proportion of each peptide can become coupled to the Sequelon arylamine membrane through acidic internal Asp and Glu residues rather than their C-terminal carboxyl group. This accounts for the apparent small releases of ³²P-radiactivity that are observed at some Asp and Glu residues. (C) The same combinations of GST-tagged wild type LKB1 (WT, lanes 1-9), or catalytically inactive (D194A, lanes 10-13) LKB1, or GST-alone (lane 14), FLAG-tagged STRADα or STRADβ, and myc-tagged MO25α or MO25β employed in FIG. 22, were tested for their ability to phosphorylate the indicated peptides (peptide concentration 200 μM). The results are expressed as the peptide kinase activity generated per mg of LKB1:STRAD:MO25 added to the assay. Results shown are average ± SD of 3 assays and are representative of at least two independent experiments.

FIG. 27 Activity of AMPK-related kinases in LKB1+/+ and LKB1^{-/-} MEFs. LKB1^{+/+} or LKB1^{-/-} MEFs were either left untreated (black bars) or stimulated with 10 mM phenformin (Phen, white bars) for 1 h. AMPKα1 and AMPK-related kinases were immunoprecipitated from the cell lysates, and in vitro kinase activity towards the AMARA peptide was measured as described in Materials and Methods (Example 4). To confirm equal expression of the kinases in each sample, cell lysates (AMPKα1, NUAK2, MARK2/3 and MARK4) or immunoprecipitated proteins (QIK, QSK, SIK and MARK1) were subjected to SDS-PAGE and western blot analysis. All AMPK-related kinases migrated with the expected molecular mass. In the case of AMPKα1, cell lysates were immunoblotted with a phospho Thr172 antibody (P-AMPK), that recognises the phosphorylated T-loop. A control immunoblot of LKB1 levels in cell lysates is also included in panel B. Results shown are average + SD of 2-4 assays and are representative of at least two independent experiments.

FIG. 28. AICA riboside does not activate AMPK-related kinases. (A) LKB1^{+/+} MEFs were either left untreated or stimulated with the indicated concentrations of phenformin (Phen) for 1 h. AMPKα1/a2 and LKB1 were immunoprecipitated from the cell lysates, and in vitro kinase activity towards the AMARA and LKBtide peptides respectively, were measured as described in Materials and Methods (Example 4). (B) To confirm equal expression of the kinases in each sample, cell lysates were subjected to SDS-PAGE and western blot analysis with the indicated antibodies. The phospho pT172 antibody (P-AMPK) recognises the phosphorylated T-loop of AMPKα1. Results are shown as the average ± SD for a triplicate assay and are representative of two independent experiments. (C) LKB1^{+/+} MEFs were either left untreated (black bars) or stimulated with 2 mM AICA riboside (AICAR, white bars) for 1 h. AMPKα1 and the indicated AMPK-related kinases were immunoprecipitated from the cell lysates, and in vitro kinase activity towards the AMARA peptide measured as described in Materials and Methods. Results are presented as % relative to the activity observed in non-stimulated cells, and are averages + SEM of two independent experiments. 100% corresponds to the following absolute activities; AMPKα1; 135 mU/mg, NUAK2; 0.25 mU/mg, QIK; 0.76 mU/mg, SIK; 2.73 mU/mg; MARK1; 0.14 mU/mg, MARK2/3; 3.5 mU/mg and MARK4; 1.6 mU/mg. (D) Immunoblotting of AMPK was performed as in A.

FIG. 29. Activity of AMPK-related kinases In HeLa cells. Control HeLa cells lacking LKB1 expression (−), or HeLa cells stably expressing wild type LKB1 (WT) or kinase inactive LKB1 (KI), were either left untreated (black bars) or stimulated with 10 mM phenformin (Phen, white bars) for Ih. AMPKα1 and AMPK-related kinases were immunoprecipitated from the cell lysates, and in vitro kinase activity towards the AMARA peptide was measured as described in Materials and Methods (Example 4). To confirm equal expression of the kinases in each sample, cell lysates (AMPKα1, MARK2/3 and MARK4) or immunoprecipitated proteins (NUAK2, QIK, QSK, SIK and MARK1) were subjected to SDS-PAGE and western immunoblot analysis. All AMPK-related kinases migrated with the expected molecular mass. In the case of AMPKα1, cell lysates were immunoblotted with a pThr172 antibody (P-AMPK), that recognises the phosphorylated T-loop. Results shown are average + SD of 2-4 assays and are representative of at least two independent experiments.

FIG. 30. Analysis of phosphorylation of BRSK2, NUAK2 and MARK4 and MELK. (A) Catalytically inactive mutants of BRSK2[D159A] and NUAK2[D193A], that are unable to autophosphorylate, were incubated with LKB1 complex for 30 min with Mg²⁺-[γ³²P]ATP and separated by electrophoresis on a polyacrylamide gel which was then autoradiographed. The ³²P-labelled proteins were digested with trypsin and the resulting peptides were chromatographed on a C₁₈ column. Fractions containing the major ³²P-labelled peptides are marked. (B) Peptides P₁, P₂ and P₃ were subjected to solid phase sequencing and ³²P-radioactivity was measured after each cycle of Edman degradation. We were unable to determine the identity of the NUAK2 peptide indicated with “?”. (C) The indicated peptides were analysed by MALDI TOF-TOF mass spectrometry as described above. The deduced amino acid sequence and the site of phosphorylation are indicated, together with the observed and theoretical mass. The peptide labelled “MARK4” was derived from MARK4 phosphorylated by LKB1 as described in the legend to FIG. 25G. This peptide encompasses the T-loop motif of MARK4 phosphorylated at only the Thr residue. The peptide labelled “MELK” is derived from a tryptic digest of unlabelled MELK that had been expressed and purified from E. coli as described above. This peptide encompasses the T-loop of MELK phosphorylated at the Thr residue. Abbreviations: m, methionine sulphone; (p) indicates preceeding Thr residue is phosphorylated.

### EXAMPLE 1

### MO25 Isoforms Interact with the STE20-Related Pseudokinases STRADα/β and Enhance Their Ability to Bind, Activate and Localise the LKB1 Tumour Suppressor in the Cytoplasm

Mutations in the LKB1 protein kinase result in the inherited Peutz Jeghers Cancer Syndrome. Research to date has indicated that LKB1 functions as a tumour suppressor protein but still little is known about how LKB1 is regulated. Recent work revealed that LKB1 can be activated through its interaction with the STE20-related, catalytically inactive pseudokinase termed STRADα and that this interaction was required for LKB1 to suppress cell growth. In this Example we have found that the endogenous LKB1-STRADα complex immunoprecipitated from a number of mammalian cells is also associated with a 40-kDa protein of unknown function, containing no identifiable functional domains, termed MO25α. We show that MO25α does not bind directly to LKB1 but instead is associated with LKB1 through its interaction with STRADα. MO25α specifically recognises the last 3 residues of STRADα (Trp-Glu-Phe) as mutation or truncation of these amino acids abolishes the interaction of STRADα with MO25α. Cellular localisation studies indicate that MO25α and STRADα form a cytoplasmic complex anchoring LKB1 in the cytoplasm, excluding it from the nucleus. The amount of LKB1 associated with STRADα in cells is markedly increased by overexpression of MO25α, suggesting that MO25α enhances the formation of the LKB1-STRADα complex in vivo. We also establish that the association of LKB1 with STRADα and MO25α stimulates the catalytic activity of LKB1 nearly 10-fold. Moreover, we provide evidence that the closely related isoform of STRADα (also known as ILPIP or PAP kinase), which we named STRADβ, and MO25β forms are also able to stabilise LKB1 in an active complex, and we demonstrate that it is possible to isolate heterotrimeric complexes of LKB1 bound to STRAD and MO25 isoforms, in which the subunits are present in equimolar amounts. Our results indicate that the MO25 may function as a scaffolding component of the LKB1-STRAD complex and play a crucial role in regulating LKB1 activity and cellular localisation mediated by its interaction with the STRAD pseudokinases.

Results

Identification of MO25α in an LKB1 Complex.

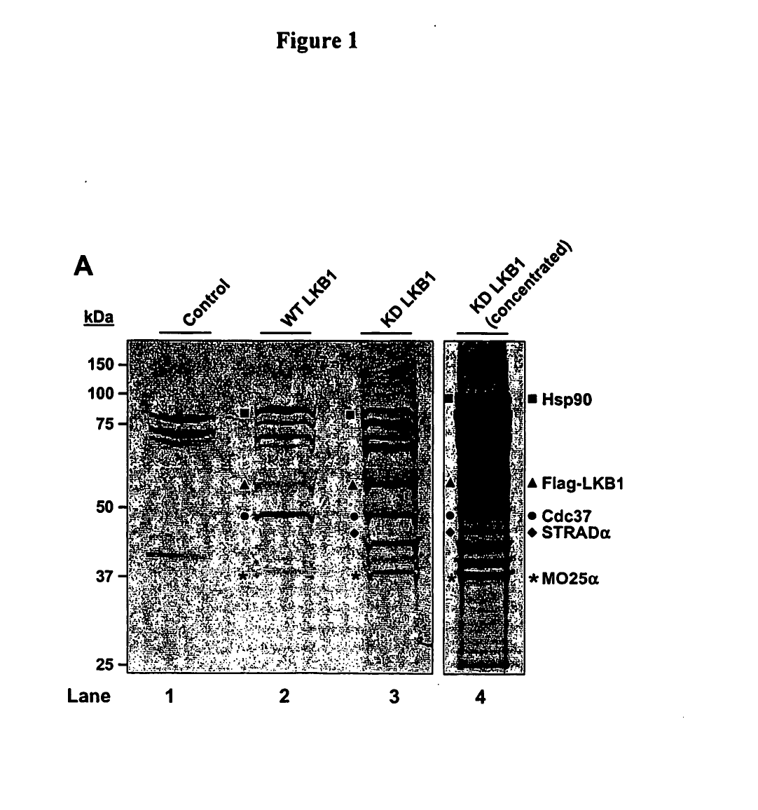

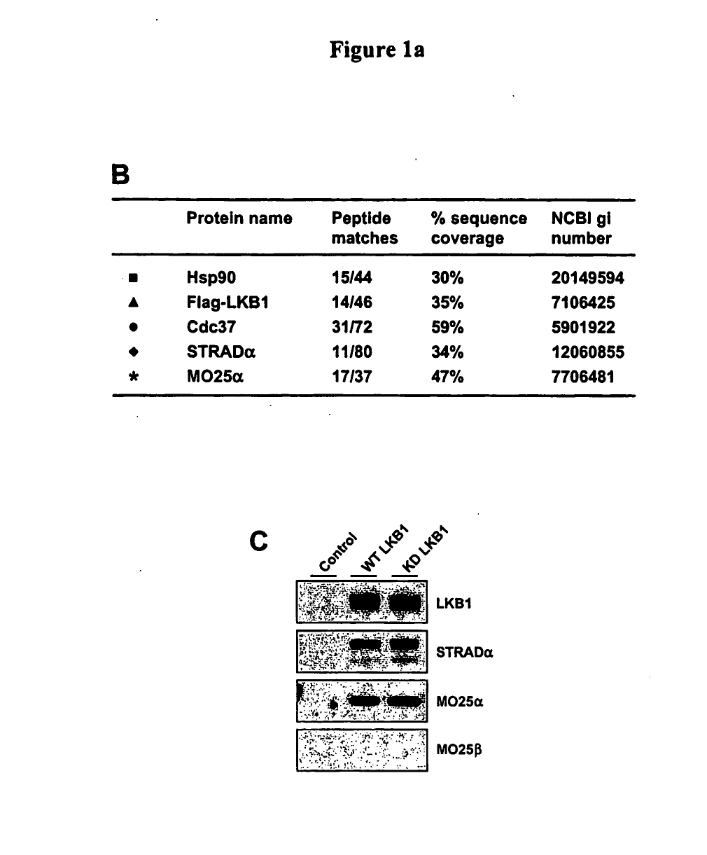

In order to identify proteins associated with LKB1, we have utilised previously described HeLa cells stably expressing low levels or either the wild type or catalytically inactive LKB1 with an N-terminal Flag epitope tag to enable facile immuno-purification of LKB1-associated proteins employing the Flag antibody (Boudeau et al., 2003a). Flag-LKB1 was immuno-purified from one hundred 10 cm dishes of HeLa cell lysate derived from the cells expressing wild type LKB1 or kinase dead LKB1, or from the control parental HeLa cell line that does not express LKB1 (see Materials and Methods). The preparations were subjected to electrophoresis on a polyacrylamide gel, which was stained with colloidal blue (FIG. 1A). A sample of the kinase dead LKB1 preparation was also concentrated further to enable better visualisation of lower abundance proteins. Several bands were observed which were present in both the wild type and kinase dead LKB1 preparations, but were absent in the control sample (FIG. 1A). The identity of the colloidal blue-stained bands labelled in FIG. 1A was established by tryptic peptide mass-spectral fingerprinting procedures (FIG. 1B), and confirmed by immunoblotting with appropriate antibodies (FIG. 1C). Proteins previously established to be associated with LKB1, including the Hsp90 and Cdc37 chaperone proteins (Boudeau et al., 2003a) and STRADα (Baas et al., 2003) were detected. In addition, a protein of ˜40 kDa identified as MO25α, co-immuno-purified with both wild type and kinase dead LKB1 but was not present in the control purification (FIGS. 1A and 1C).

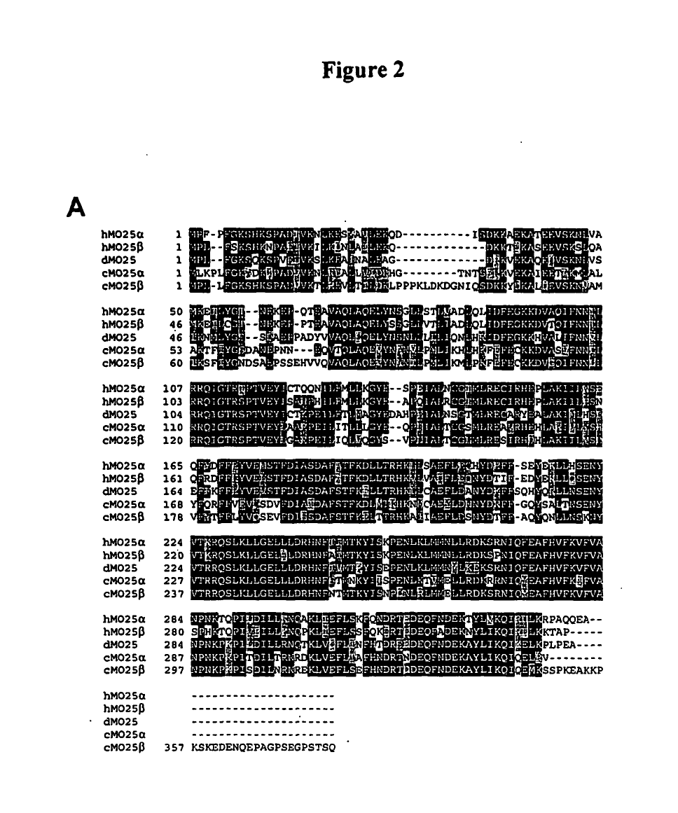

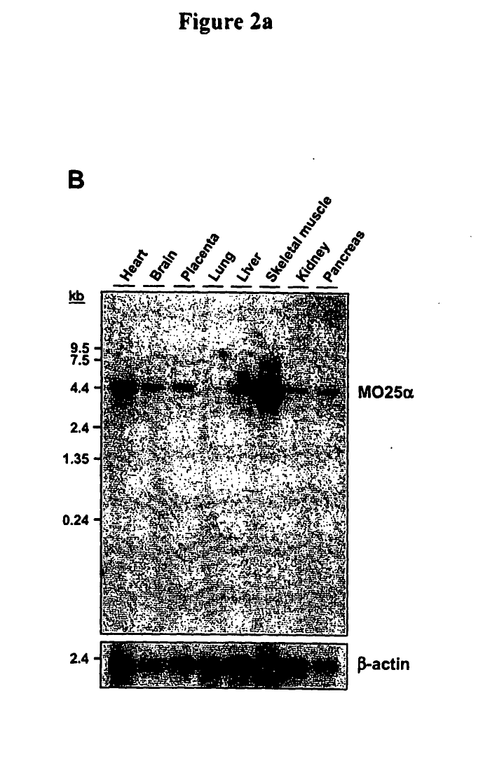

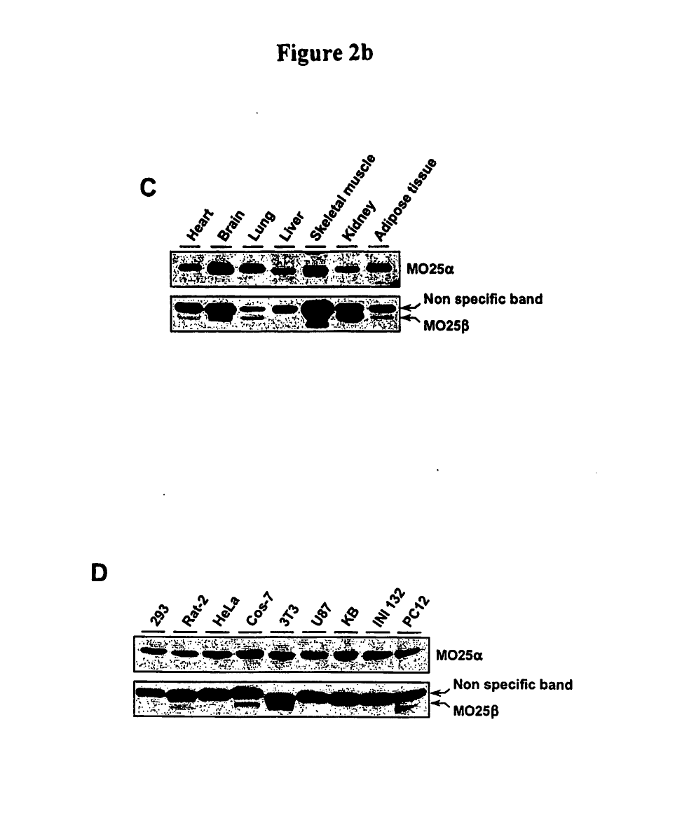

MO25α was first identified as a gene expressed at the early cleavage stage of mouse embryogenesis (Miyamoto et al., 1993) and was shown to be a highly evolutionary conserved protein (Karos and Fischer, 1999; Nozaki et al., 1996) for which no cellular function has been ascribed. From analysis of databases, we have identified a closely related isoform of MO25α, which we termed MO25β, and a sequence alignment of both MO25 isoforms together with their putative homologues in Drosophila and C. elegans is shown in FIG. 2A. Northern blot analysis indicated that MO25α is widely expressed in human tissues, with highest levels of expression in skeletal muscle (FIG. 2B). Immunoblot analysis using an antibody raised against the MO25α protein, which does not cross react with MO25β (FIGS. 2C & D), and analysis of EST databases (Table 1) confirmed that MO25α is expressed in many tissues and cell lines. Although we were unable to detect significant levels of MO25β RNA by Northern blot analysis, immunoblotting using an antibody raised against the MO25β protein, which does not cross react with MO25α, suggested that MO25β is also expressed in a variety of tissues and cell lines tested (FIG. 2D). This is consistent with the observation that MO25β EST sequences have been identified from a number of human tissues (Table 1). However, expression of MO25β may be more restricted than MO25α, as it was not detected in liver and several cell lines including HeLa cells (FIG. 2D), perhaps explaining why it was not co-purified with LKB1 expressed in these cells (FIG. 1).

TABLE 1
Tissue origin of ESTs encoding human M025α and M025β

| Protein | | Tissue NCB1 Accession |
|---|---|---|
| MO25α | duodenum, | NM_016289 |
| | adenocarcinoma | |
| | leiomyosarcoma | BM474393, BU167673, BE884976 |
| | embryonal | BQ433499, BM905560, BG259357 |
| | carcinoma | |
| | squamous cell | BG679680 |
| | carcinoma | |
| | melanotic melanoma | BQ432236 |
| | mammary | BM013638, BG034567 |
| | adenocarcinoma | |
| | chronic lymphotic | AI760873 |
| | leukemia | |
| | ductal carcinoma | BU190932 |
| | osteosarcoma | BG108474 |
| | adenocarcinoma | BG250662, BG121835, AW188515 |
| | metastatic | CA436298 |
| | chondrosarcoma | |
| | epithelioid | BQ433038 |
| | carcinoma | |
| | retinoblastoma | BM480142 |
| | neuroblastoma | BE383169 |
| | uterus, tumor | BF095794 |
| | melanotic melanoma | BE891921, BQ231688 |
| | astrocytoma | BM913690, BM560554 |
| | transitional | G260085 |
| | cell papilloma | |
| | skin tumor | AA379224 |
| | testis | BG773506, BG719459 |
| | sympathetic trunk | BQ718408 |
| | marrow | BI019682, BI019684 |
| | normal pigmented | BG750933, BM050770 |
| | retinal epithelium | |
| | cervix | BI089979 |
| | hypothalamus | AV722015, AV729226 |
| | fetal brain | BM927264, AA351866 |
| | senescent | W47588 |
| | fibroblasts | |
| | infant brain | AA434013, , R21390, R46486 |
| | germinal center | AA280522, AA280264, AA262054 |
| | B cell | |
| | ovarian tumor | AA442950, AA165271, AA164403 |
| | placenta | R67490, R66347, BG435220, |
| | osteosarcoma | BG025988 |
| | breast, normal | BE004664, AW373436 |
| | fetal liver | T86658, T86848 |
| | spleen | |
| | cervix | BE538532 |
| | stomach | BM822360, BM833391, BM766941 |
| | fetal eyes | BM711209 |
| | purified | BU079122, BU078843 |
| | pancreatic islet | |
| | prostate, normal | BE835801 |
| | lung | BM979124, BM982100, BM985320 |
| | kidney | AW235071, AA961656 |
| MO25β | prostate | BF680686 |
| | placenta, | BC010993, BE281207, BF107447 |
| | choriocarcinoma | |
| | teratocarcinoma | AU125107, AU148840, AK022639 |
| | parathyroid, tumor | W37952 |
| | sciatic nerve | BQ899617 |
| | germinal center | AA278473, AA279145 |
| | B cell | |
| | lung tumor | BF879769 |
| | epidermoid | BQ669953 |
| | carcinoma | |
| | lung, normal | BF845952, BE042886, |
| | colon | BE928225, AW008806 |
| | metastatic | BQ021348 |
| | chondrosarcoma | |
| | fibrosarcoma | CA441154 |
| | kidney | AW242839 |
| | stomach | BM820071, BM831835, BM822014 |
| | liver and spleen | AI247543 |
| | testis | AA781806 |
| | pancreas | AI956166 |
| | retina | AA001436, AA018841 |

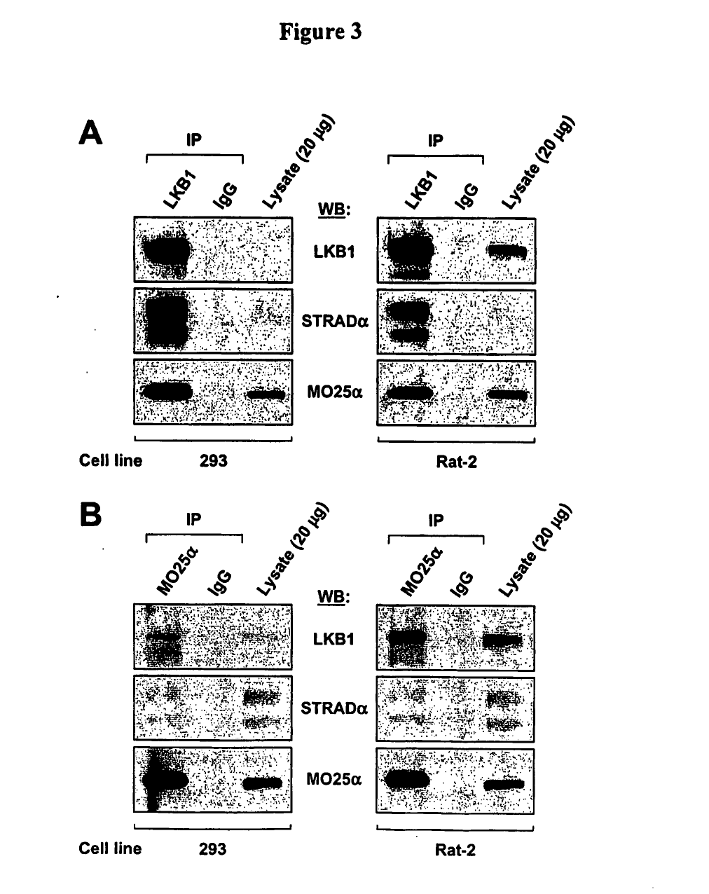

Endogenous MO25α is present in complex with endogenous LKB1. We next immunoprecipitated endogenous LKB1 from 293 cells (FIG. 3A, left panel) or Rat-2 cells (FIG. 3A, right panel), followed by immunoblotting for LKB1, STRADα and MO25α. The experiments showed that MO25α, as well as STRADα, were co-immunoprecipitated with LKB1, but not with preimmune IgG. We also immunoprecipitated endogenous MO25α from 293 cells (FIG. 3B, left panel) or Rat-2 cells (FIG. 3B, right panel), and immunoblotted for the presence of LKB1, STRADα and MO25α (FIG. 3B). Endogenous LKB1 and STRADα were co-immunoprecipitated with MO25α, but not with preimmune IgG, consistent with the notion that these proteins form a complex.

MO25 Interacts with STRAD Rather than with LKB1.

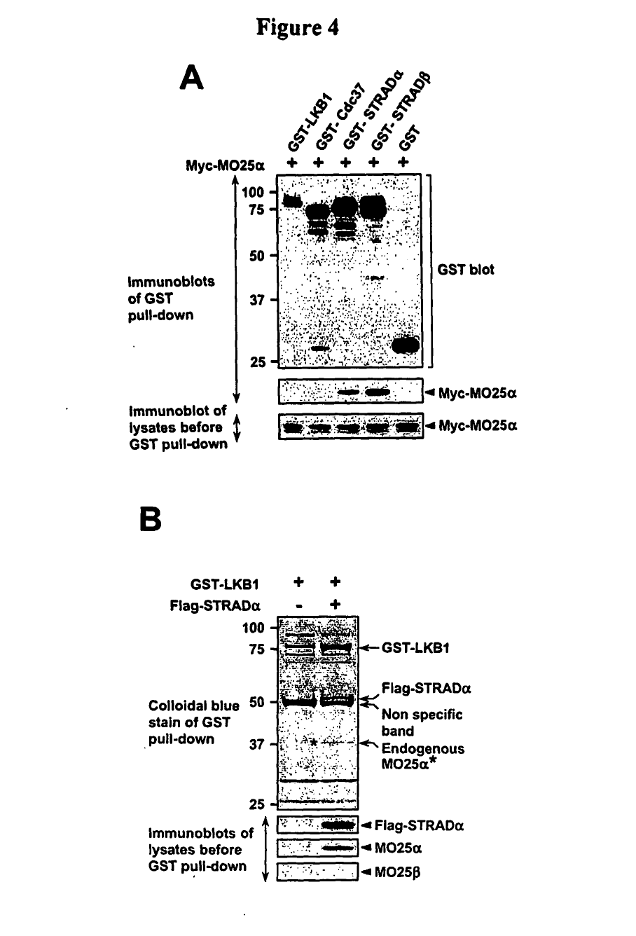

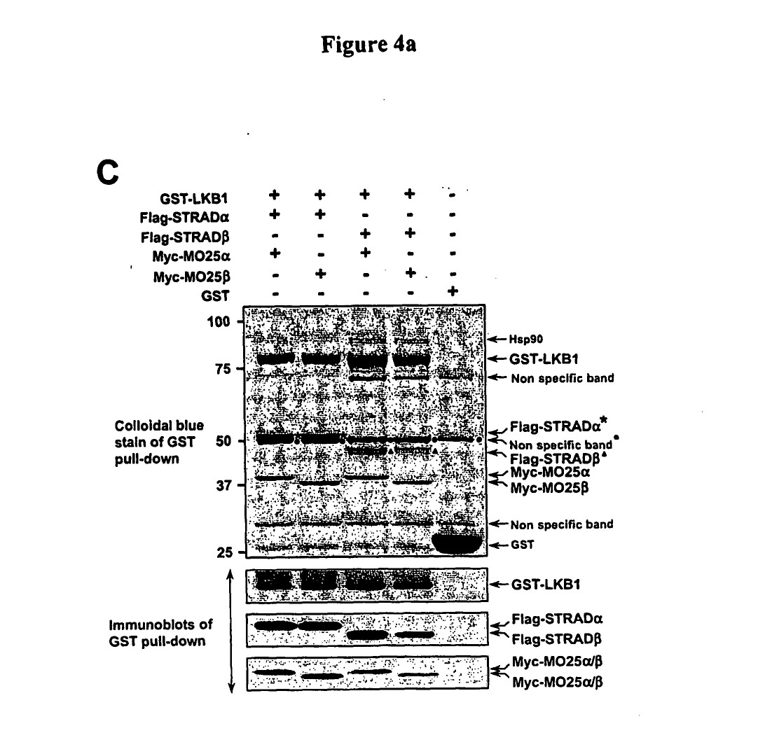

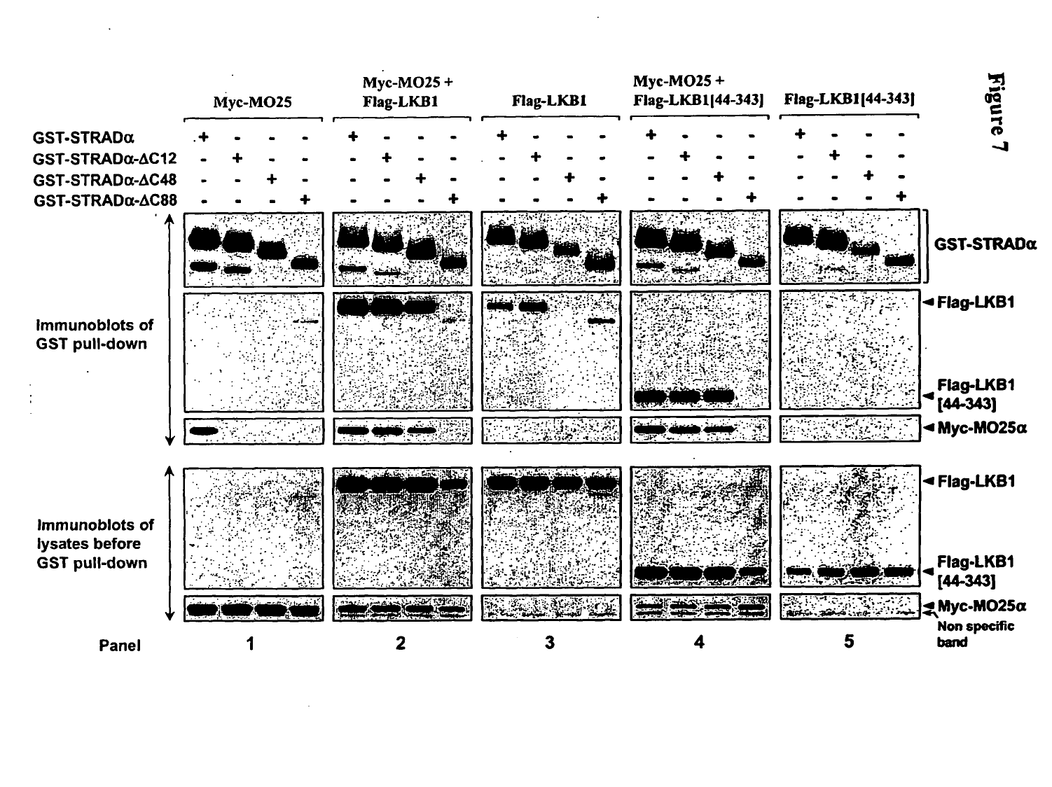

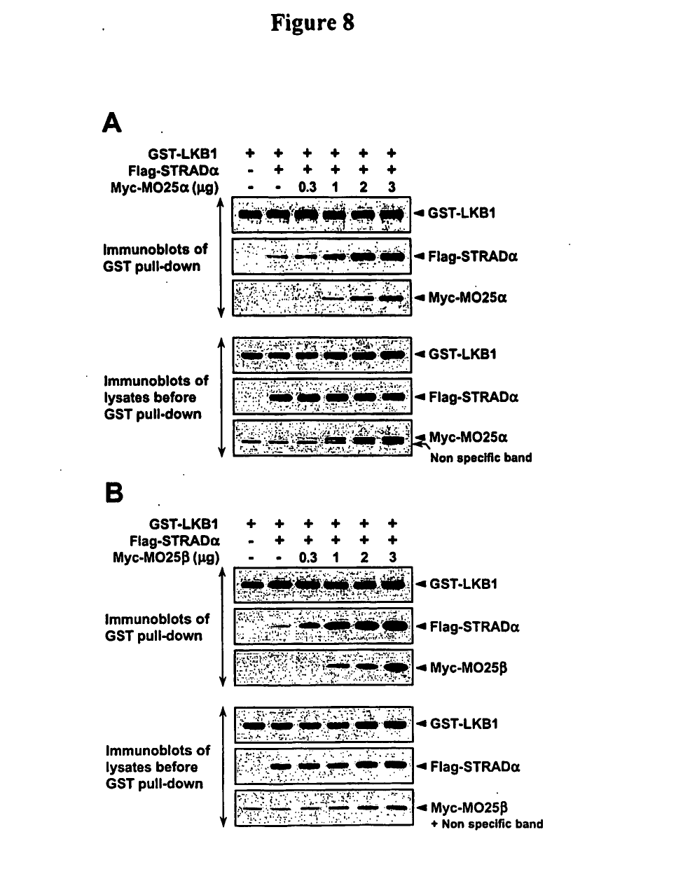

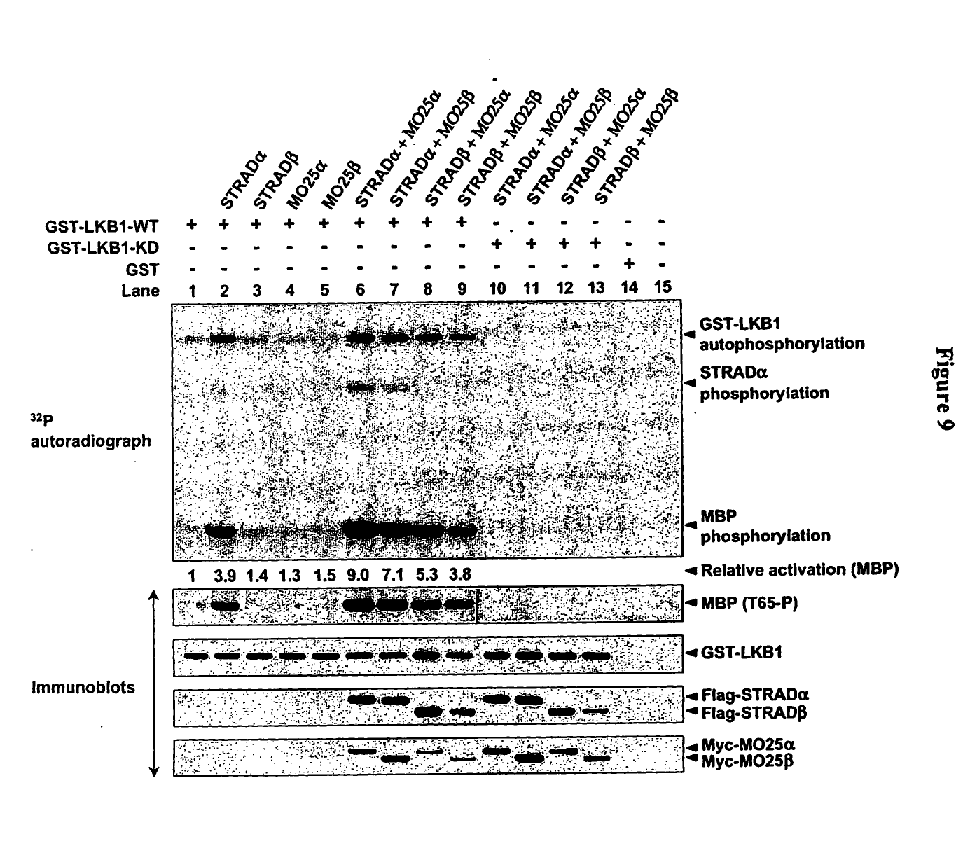

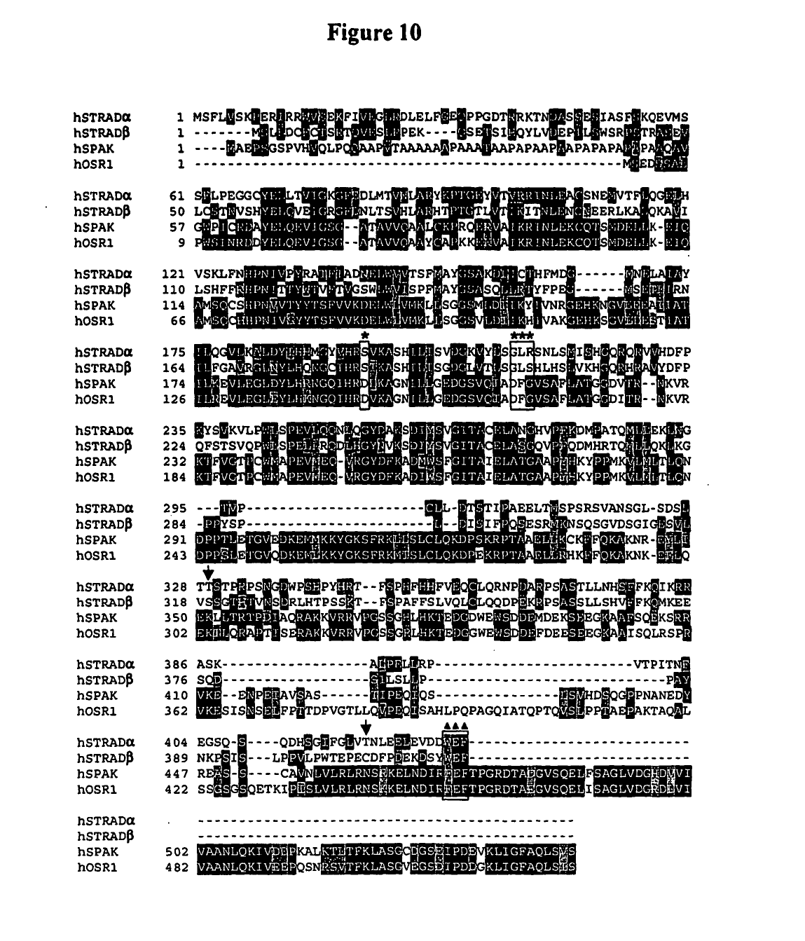

To verify which component of the LKB1-STRAD complex MO25α interacted with, we co-transfected 293 cells with constructs expressing MO25α and either N-terminal glutathione S-transferase (GST) tagged LKB1, STRADα or the closely related STRADβ pseudokinase, as well as Cdc37, a chaperone previously shown to bind LKB1 (Boudeau et al., 2003a). A sequence alignment of STRADα and STRADβ is shown in the FIG. 10 and both lack the same key conserved residues in subdomain VI and VII in the kinase domain that are required for catalysis. The GST-tagged proteins were affinity purified and immunoblotted for MO25α (FIG. 4A). We found that MO25α interacts only with STRADα and STRADβ but not with LKB1 or Cdc37.

We next expressed LKB1 in 293 cells in the presence or absence of STRADα, and the protein composition of the affinity purified LKB1 was analysed following electrophoresis and staining with colloidal blue. As expected, STRADα was co-purified with LKB1. However, in addition, a faintly staining band corresponding to an endogenous protein of ˜40 kDa was observed in the LKB1-STRADα complex, which was not present in the uncomplexed LKB1 preparation. Tryptic peptide mass spectral fingerprinting revealed that this protein corresponded to endogenous MO25α. This was confirmed by immunoblotting with an anti-MO25α antibody (FIG. 4B), further suggesting that MO25α is associated with LKB1 through STRADα. In FIG. 4C, we demonstrate by performing appropriate co-transfections, that it is possible to purify a heterotrimeric LKB1 complex in which LKB1, STRADα and MO25α are present in equivalent equimolar proportions. We also demonstrate that it is possible to form similar complexes of LKB1-STRADα-MO25β, LKB1-STRADβ-MO25α and LKB1-STRADβ-MO25β (FIG. 4C), indicating that both isoforms of STRAD and MO25 are able to bind each other, as well as LKB1.

MO25α Cooperates with STRADα to Localise LKB1 in the Cytoplasm.

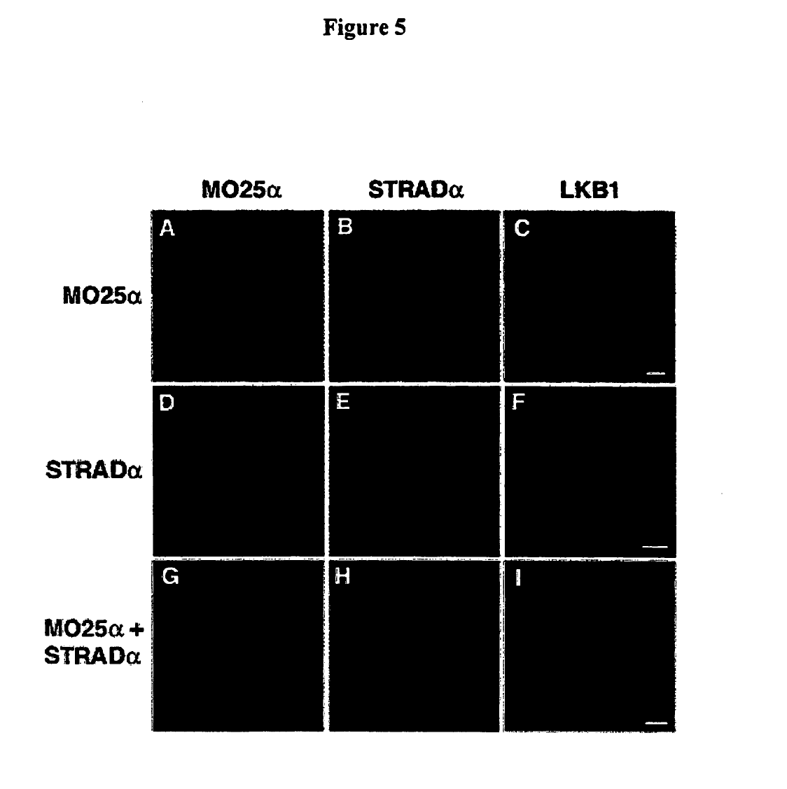

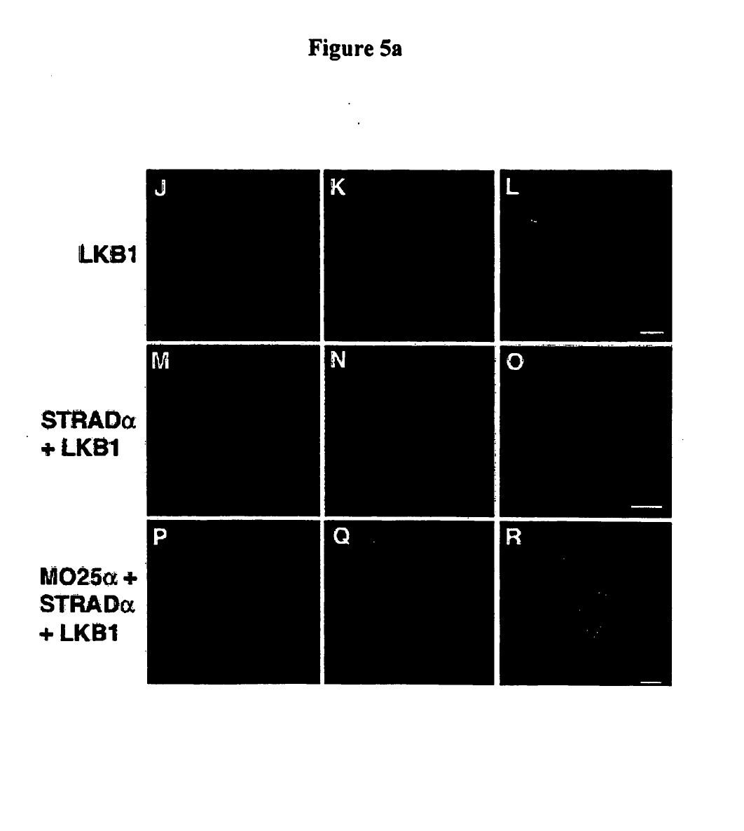

The cytoplasmic fraction of LKB1 was recently shown to conduct a G1 cell arrest (Tiainen et al., 2002). Therefore, we were interested in studying how the formation of a complex of LKB1, STRADα and MO25α affected the localisation of these proteins. HeLa cells were transfected with combinations of tagged MO25α, STRADα and LKB1 and these proteins were visualised in cells by confocal fluorescence microscopy in which the MO25α, STRADα and LKB1 proteins could be detected independently in the same cell. MO25α (FIG. 5A) and STRADα (FIG. SE) expressed on their own were localised throughout the cytoplasm and nucleus. Strikingly however, when MO25α and STRADα were co-expressed, both proteins were re-localised to the cytoplasm and were excluded from the nucleus (FIGS. 5G & 5H). As reported previously in COS cells, LKB1 expressed alone was mainly nuclear (FIG. 5L) and co-expression with STRADα promoted significant cytoplasmic localisation of LKB1, however significant levels of LKB1 remained in the nucleus under these conditions (FIG. 50) (Baas et al., 2003). In the presence of both MO25α and STRADα however, LKB1 was essentially only localised in the cytoplasm and virtually excluded from the nucleus (FIG. 5R). These observations indicate that MO25α and STRADα form a complex that anchors LKB1 in the cytoplasm more effectively than STRADα alone.

MO25α Recognises the Last 3 Residues of STRADα.

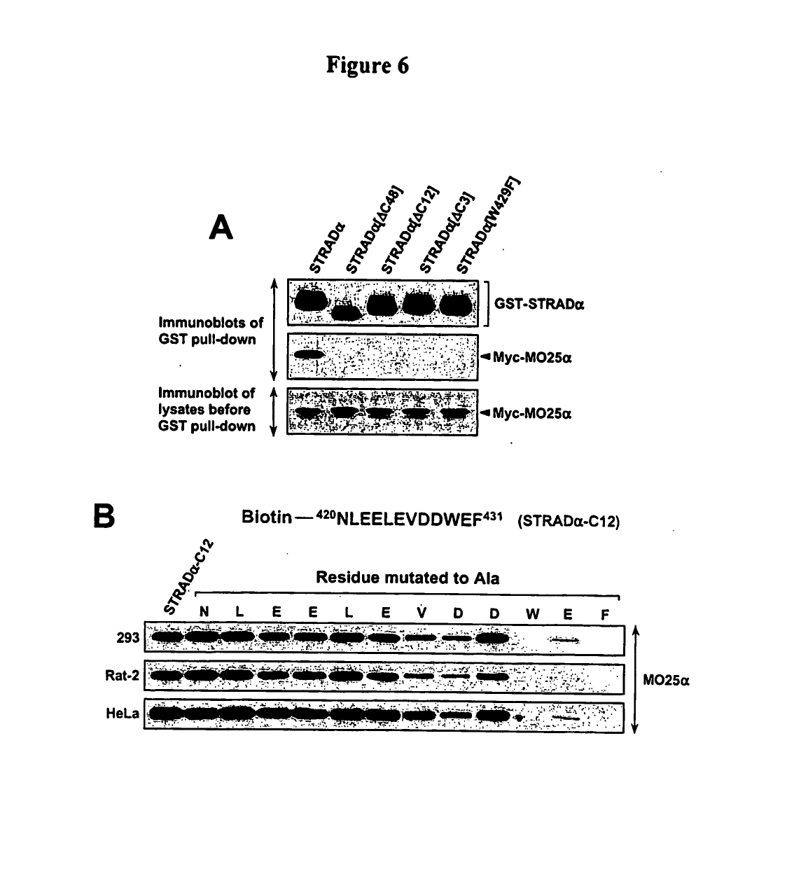

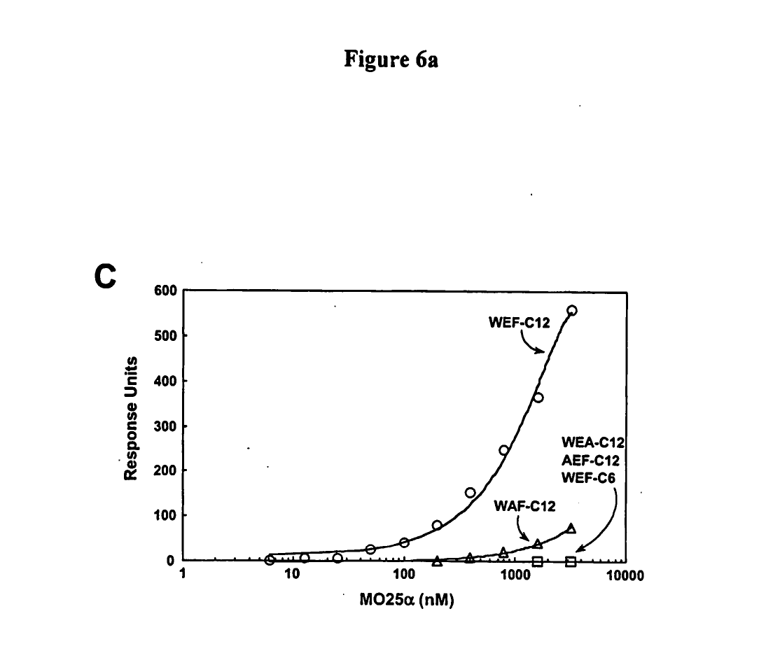

To define the binding site of MO25α on STRADα, a series of deletion mutations of STRADα were tested for their ability to interact with MO25α in a co-transfection binding assay. Strikingly, deletion of only the last 3 amino acids of STRADα (Trp-Glu-Phe) abolished binding of MO25α to STRADα. Moreover, mutation of the C-terminal Trp residue to Phe, also vastly reduced binding of MO25α to STRADα (FIG. 6A). We then tested whether it was possible to affinity purify endogenously expressed MO25α from 3 different mammalian cell lysates employing biotinylated peptides encompassing the C-terminal residues of STRADα conjugated to streptavidin-Sepharose. We found that a peptide encompassing the C-terminal 12 residues (FIG. 6B) but not the last 6 residues (data not shown and FIG. 6C) of STRADα efficiently purified endogenous MO25α from all cell lysates tested (FIG. 6B). To further delineate the MO25α binding site on the STRADα peptide, we performed an alanine scan, mutating each residue of the peptide individually to Ala and verifying how this affected binding to MO25α (FIG. 6B). Consistent with the Trp-Glu-Phe residues being required for MO25α recognition, mutation of any of these 3 residues in the C-terminal STRADα peptide abolished or vastly reduced MO25α binding in 3 different cell lysates, whereas mutation of the other residues either did not affect binding or only had a moderate effect. These findings were also confirmed in a more quantitative surface plasmon resonance Biacore binding assay (FIG. 6C).

Binding of LKB1 to STRADα Creates Novel Binding Site(s) for MO25α.

We next investigated how binding of LKB1 to STRADα c affected the interaction with MO25α using a co-expression binding assay in 293 cells in which full length wild type and deletion mutants of GST-STRADα were co-expressed in the presence or absence of MO25α and LKB1. Consistent with previous findings, STRADα mutants that lack either the C-terminal 12 or 48 residues did not bind MO25α in the absence of LKB1 (FIG. 7, panel 1). Strikingly though, when these STRADα mutants were co-expressed with LKB1, MO25α was recovered in the purified complexes (FIG. 7, panel 2), indicating that the binding of LKB1 to STRADα generates additional binding site(s) for MO25α within the LKB1-STRADα complex. We also consistently noticed that the amount of LKB1 recovered in the GST-STRADα pull downs was significantly higher in the presence of MO25α (FIG. 7, compare panels 2 and 3), suggesting that MO25α might stabilise the formation of a heterotrimeric LKB1 complex. Moreover, a catalytic fragment of LKB1 encompassing only the kinase domain (residues 44-343) interacted much more efficiently with STRADα in the presence of MO25α (FIG. 7, compare panels 4 and 5). These observations indicate that MO25α could be stabilising the LKB1-STRADα complex by generating additional MO25α binding site(s) on STRADα and/or LKB1. However, a mutant of STRADα lacking the C-terminal 88 residues that truncates a region of the pseudokinase domain, was no longer able to interact with LKB1 or MO25α when these proteins were co-expressed (FIG. 7). This suggested that either a secondary MO25α binding site is located towards the C-terminus of STRADα or that the integrity of the pseudokinase domain, which could be disrupted by truncation of the 88 C-terminal residues of STRADα, is required in enabling formation of the LKB1-STRADα-MO25α heterotrimeric complex.

Further Evidence that MO25 Stabilises the LKB1-STRAD Complex.

To obtain further evidence that MO25α could stabilise the binding of LKB1 to STRADα, 293 cells were co-transfected with constructs expressing LKB1 and STRADα in the absence or presence of increasing amounts of the MO25α DNA construct. LKB1 was purified and the levels of associated STRADα and MO25α were measured by immunoblotting. As a control, the levels of expression of LKB1, STRADα and MO25α were also measured in the cell lysates prior to affinity purification of LKB1. In the absence of MO25α, a moderate amount of STRADα was associated with LKB1, which was markedly enhanced by increasing expression of MO25α in a dose dependent manner (FIG. 8A, upper panel). Increased association of STRADα with LKB1 was not due to increased expression of LKB1 or STRADα as these were expressed in cell lysates at similar levels in the absence or presence of increasing amounts of MO25α (FIG. 8A, lower panel). MO25β was similarly able to enhance the interaction between LKB1 and STRADα (FIG. 8B).

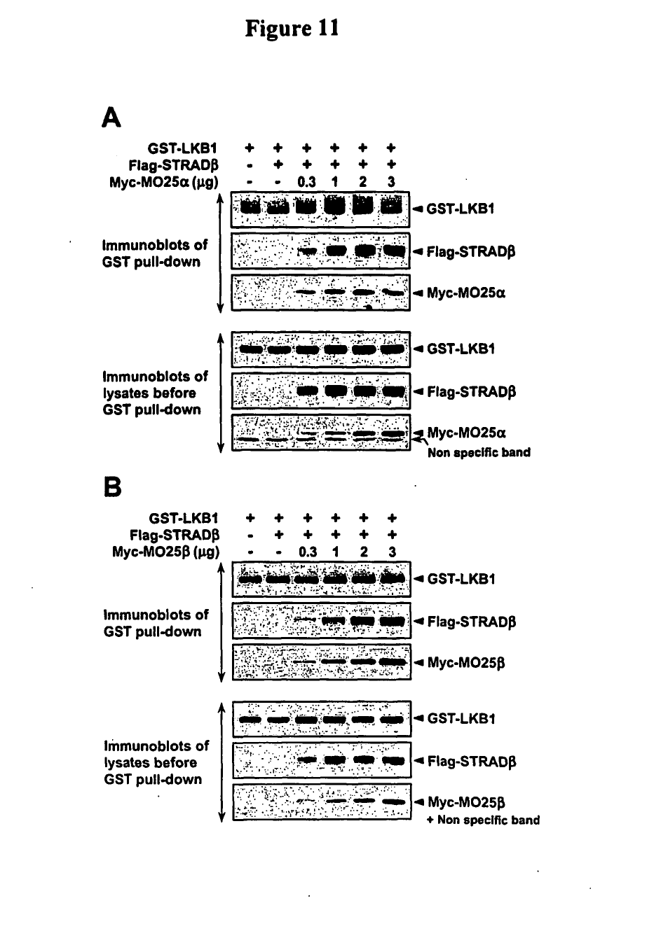

We also investigated how MO25α and MO25β stabilised the binding of LKB1 to STRADβ (FIG. 11). No detectable association of STRADβ was observed to LKB1 in the absence of MO25, whereas co-expression of MO25α or MO25β resulted in a dose dependent increase in STRADβ binding to LKB1. However, in contrast to STRADα, the levels of STRADβ expression in 293 cells were low in the absence of MO25 isoforms and significantly increased in their presence, which could explain the increased binding of LKB1 to STRADβ in the presence of MO25 isoforms (Supplementary FIG. 2). Similar results were obtained when STRADβ was expressed as a GST-fusion protein from a distinct expression vector (data not shown).

Stabilisation of the LKB1-STRAD Complex by MO25 Isoforms Correlates with Increased Activity.

To determine how the binding of MO25α and MO25β to the LKB1-STRADα or LKB1-STRADβ complex affected LKB1 activity, 293 cells were transfected with wild type GST-LKB1 in the presence or absence of STRAD and/or MO25 isoforms. The activity of equal amounts of affinity purified GST-LKB1 was evaluated by measuring autophosphorylation activity as well as the transphosphorylation of myelin basic protein (MBP), a non-specific exogenous substrate of LKB1, in the presence of [γ-³²P]ATP. We also monitored MBP phosphorylation using a phospho-specific antibody that recognises the major LKB1 phosphorylation site on MBP (Thr65) (Baas et al., 2003). The different LKB1 activity assays gave comparable results (FIG. 9). In the absence of STRAD isoforms, LKB1 was poorly active (FIG. 9, lane 1) and, as shown previously (Baas et al., 2003), co-expression with STRADα induced an 4-fold increase in LKB1 activity (FIG. 9, lane 2). In the presence of STRADα and either MO25α or MO25β, LKB1 activity was increased a further 2-fold so that LKB1 became ˜9-fold more active than LKB1 expressed alone (FIG. 9, lanes 6 or 7). LKB1 was only activated by STRADβ in the presence of MO25 isoforms (FIG. 9, compare lanes 3 with lanes 8 & 9), consistent with the observation that LKB1 is barely associated with STRADβ in the absence of MO25 (FIG. 11). LKB1 was previously shown to phosphorylate STRADα (Baas et al., 2003), and we show that the MO25-mediated increase in the association of STRADα to LKB1 results in increased STRADα phosphorylation (FIG. 9, compare lanes 2 with 6 & 7). In contrast to STRADα, STRADβ was not significantly phosphorylated by LKB1 in the presence of MO25 (FIG. 9, lanes 8 & 9). LKB1 phosphorylates STRADα at Thr329 and Thr419 (Baas et al., 2003), sites that are not conserved in STRADβ (FIG. 10). This explains why STRADβ is not phosphorylated by LKB1. We also showed that catalytically inactive LKB1 is able to associate with both STRAD isoforms in the presence of either isoform of MO25. However, as expected, it is unable to autophosphorylate or phosphorylate STRADα or MBP (FIG. 9, lanes 10-13).

Discussion.

One of the major findings of this study is the discovery that the highly conserved MO25α protein found in many eukaryotic species, for which no function had been ascribed to previously, is present in a complex with STRADα and LKB1 in vivo. Interestingly, in the absence of LKB1, MO25α specifically recognises the last 3 amino acids of STRADα that lie in a Trp-Glu-Phe sequence, and mutation of any of these 3 residues to Ala essentially prevented the binding of MO25α to STRADα. In this regard, MO25α functions similarly to a PDZ domain, which also recognises the extreme C-terminal residues of its protein binding partners (Songyang et al., 1997). However, there is no homology between a PDZ domain and MO25α and, to our knowledge, other domains characterised thus far do not possess this property. The C-terminal non-pseudokinase domain ˜50 residues of STRADα and STRADβ are poorly conserved but, significantly, both terminate with the Trp-Glu-Phe sequence (FIG. 10), emphasising that STRADα and STRADβ have probably evolved to interact specifically with MO25 isoforms. The most closely related proteins to STRADα and STRADβ are the active STE20 member kinases termed SPAK (Johnston et al., 2000) and OSR1 (Tamari et al., 1999), which possess all residues required for protein kinase catalysis (FIG. 10) and are therefore active enzymes. Although neither SPAK nor OSR1 terminate in a Trp-Glu-Phe sequence, both possess a Phe-Glu-Phe sequence at a similar region of their C-terminal non-catalytic domain (FIG. 10). This observation prompted us to mutate the C-terminus Trp-Glu-Phe sequence of STRADα to Phe-Glu-Phe and we found that this prevented MO25α from binding to STRADα (FIG. 6A). This further emphasizes that the presence of a Trp residue three residues from the end of the protein is essential for binding of STRADα to MO25α, suggesting that MO25α will not interact with SPAK or OSR1.

Our data indicate that one of the key roles of MO25α is to stabilise the interaction between LKB1 and STRADα. This is based on the observation that the amount of STRADα associated with LKB1 in cells is significantly enhanced by the expression of MO25α (FIG. 8). The finding that STRADα lacking the C-terminal Trp-Glu-Phe residues can form a complex with MO25α in the presence of LKB1 (FIG. 7) indicates that the interaction of LKB1 with STRADα generates novel binding site(s) for MO25α, which could explain how MO25α stabilises the association of LKB1 with STRADα. Our findings do not enable us to discriminate whether the additional MO25α binding site(s) are located within STRADα and/or LKB1. Detailed structural studies of this complex will be required to address this issue. Our observations are consistent with the notion that MO25α or MO25β may function as a scaffolding component of the LKB1-STRAD complex.

It has previously been shown that co-expression of STRADα with LKB1 enhanced LKB1 in vitro activity (Baas et al., 2003) and here we showed that the association of LKB1-STRAD with MO25 isoforms further enhanced LKB1 activity 2-fold (FIG. 9). These results indicate that stabilisation of the LKB1-STRADα complex by MO25 is accompanied by activation of LKB1. Importantly, however, it should be noted that the complex of LKB1 and STRADα purified from mammalian 293 cells overexpressing tagged LKB1 and STRADα is also associated with significant levels of endogenous MO25α (FIG. 4B). It is therefore not possible to measure the activity of the LKB1-STRADα complex in the absence of MO25α by expression of these proteins in mammalian cells. Thus we cannot quantify how association of STRADα with LKB1 in the complete absence of MO25α affects LKB1 activity. To overcome the problem of association of overexpressed STRADα with endogenous MO25α in mammalian cells, we have attempted to combine E. coli expressed LKB1, STRADα and MO25α to see whether we could form a complex in which LKB1 could be activated. However, this approach was unsuccessful (JB data not shown), which indicate that the association of LKB1 with STRADα and MO25α is a complicated process that may require additional factors and/or that this complex may need to be assembled in vivo. We were able to isolate from mammalian cells a heterotrimeric complex in which LKB1, STRAD and MO25 subunits are present at a similar stoichiometry (FIG. 4C), suggesting a relatively high affinity of the individual subunits for each other.

We also demonstrated that MO25α and STRADα, when expressed alone in cells, are both localised in the cytoplasm and nucleus, but they are only localised in the cytoplasm and excluded from the nucleus when expressed together (FIG. 5). The molecular mechanism underlying this observation has not been investigated. It is possible that the interaction of MO25α and STRADα leads to the masking of a nuclear localisation signal, exposure of a novel nuclear export signal or cytoplasmic anchoring motif, or that the STRADα-MO25α complex is to large to freely translocate into the nucleus. Under the conditions we have performed our localisation studies in HeLa cells, we found that MO25α significantly cooperates with STRADα to localise LKB1 in the cytoplasm of cells, excluding it from the nucleus, as the nuclear exclusion and cytoplasmic localisation of LKB1 observed following expression of LKB1 and STRADα was markedly enhanced in the presence of MO25α (FIG. 5). Previous studies in COS cells also showed that STRADα could localise LKB1 in cytoplasm of cells (Baas et al., 2003). However, as our data indicate that significant levels of endogenous MO25α will bind to the LKB1-STRADα complex (FIG. 4B), it is not possible to rule out that endogenous MO25 isoforms could contribute to cytoplasmic localisation of LKB1 when it is co-transfected with STRADα. Cytoplasmic localisation of LKB1 may be important, as mutants of LKB1 that can not enter the nucleus still suppress cell growth (Tiainen et al., 2002). Additional mechanisms may also exist to maintain LKB1 in the cell cytoplasm. The interaction of LKB1 with the LIP1 protein has been shown to induce LKB1 cytoplasmic localisation (Smith et al., 2001). It has yet to be tested whether LIP1 can interact with the heterotrimeric LKB1-STRAD-MO25 complex, but LIP1 has not been reported to influence LKB1 activity. LKB1 is also prenylated at its C-terminus (Collins et al., 2000; Sapkota et al., 2001), which could promote interactions with cell membranes. Recent work has indicated that prenylation of the Drosophila LKB1 homologue is important for association of LKB1 with membranes in controlling cell polarity (Martin and St Johnston, 2003).

Putative MO25 homologues are found not only in mammals, Drosophila and C. elegans but also found in fission yeast (NCBI accession number NP_{—}594685, 51% identity to human MO25α), budding yeast (HYM1, NCBI accession number P32464, 33% identity to human MO25α) and plant (NCBI accession number Q9M0M4, 43% identity to human MO25α). To our knowledge only the putative budding yeast MO25 homologue termed HYM1, has been investigated and apparently plays a key role in regulating polarized cell growth and cell separation (Bidlingmaier et al., 2001; Dorland et al., 2000; Jorgensen et al., 2002). To our knowledge, LKB1 and STRAD homologues have not been reported in yeast and thus it is not clear whether there could be a link between LKB1 regulating polarity in C. elegans and Drosophila (Martin and St Johnston, 2003; Watts et al., 2000) and HYM1 regulating cell polarity in yeast.

It is also possible that MO25 could have functions that are independent of its ability to regulate LKB1. MO25 could interact with other proteins terminating in a similar motif to STRADα/STRADβ and there are over 20 human proteins that terminate in a Trp-Glu/Asp-Phe motif. It may be interesting to investigate whether any of these bind MO25 isoforms. It is also probable that STRADα and STRADβ have functions other than regulating LKB1. In this regard, recent work has shown that overexpression of STRADβ in 293 cells protected them against apoptosis by enhancing the ability of the JNK pathway to be activated by overexpression of XIAP, an upstream component of the JNK pathway (Sanna et al., 2002). Moreover, in another study, overexpression of STRADβ was reported to lead to activation of JNK and p38γ MAP kinases (Nishigaki et al., 2003). The mechanism by which STRADβ enhanced JNK/p38γ activation was not defined in these studies and it would be of interest to test whether LKB1 and/or MO25 is involved in these processes.

A significant number of inherited forms of PJS found in certain families do not exhibit mutations in the LKB1 genes (Buchet-Poyau et al., 2002), indicating that there is a second causative locus for PJS. Genetic analysis to date has excluded LIP1 and provided no evidence that other proteins reported to interact with LKB1 are mutated in these patients (Buchet-Poyau et al., 2002; Resta et al., 2002). The results in this study indicate that it would be worthwhile to verify whether mutations in the genes encoding STRAD or MO25 isoforms occur in any of the PJS families that do not have mutations in the LKB1 gene.

In conclusion, we define MO25 as a novel component of the LKB1 complex in vivo. Our results indicate that isoforms of MO25 function to stabilise the interaction of LKB1 with the catalytically inactive STRAD pseudokinase isoforms. This markedly enhances LKB1 activity and its cytoplasmic localisation. In future studies to further define the physiological functions of the MO25 and STRAD homologues in regulating LKB1 function, it would also be important to generate mice lacking these genes. The ability to express more active forms of LKB1 heterotrimeric complexes also opens up the opportunity to employ biochemical approaches to search for novel LKB1 substrates.

Materials. Protease-inhibitor cocktail tablets were obtained from Roche. Dimethyl pimelimidate and the anti-Flag M2-agarose affinity gel were purchased from Sigma. Tetracyclin-free foetal bovine sera (FBS) was from Perbio, and other tissue culture reagents were from Biowhittaker unless otherwise stated. Precast SDS/4-12% and 10% polyacrylamide Bis-Tris gels and myelin basic protein (MBP) were obtained from Invitrogen. Sensor chips SA were from BiaCore AB (Stevenage, UK). [γ-³²P]ATP, Glutathione-Sepharose, Protein G-Sepharose, and Streptavidin-Sepharose High Performance were purchased from Amersham Biosciences.

Peptides. The biotinylated peptides STRADα-C12 (Biotin-C6spacer-NLEELEVDDWEF), and STRADα-C6 (Biotin-C6spacer-VDDWEF) whose sequences encompass the last 12, and 6 residues of STRADα respectively, the Flag peptide (DYKDDDDK), and peptides used to raise antibodies were synthesised by Dr G. Bloomberg (University of Bristol, UK). The biotinylated peptides encompassing the last 12 residues of STRADα in which each residue was individually replaced by an Alanine were synthesized using previously described methodology (Lizcano et al., 2002).

Antibodies. The LKB1 antibody used for immunoblotting was raised against the mouse LKB1 protein as described previously (Sapkota et al., 2001). As this antibody does not immunoprecipitate efficiently the human LKB1 protein, we raised an antibody in sheep against the human LKB1 protein expressed as a GST-fusion protein in E. coli, that efficiently immunoprecipitated human and rat LKB1. This antibody was used for all the immunoprecipitating experiments in this study. The anti-MO25α and anti-MO25β antibodies were raised in sheep against the MO25α and MO25β human proteins expressed in E. coli as described below. The LKB1 and MO25 antibodies were affinity-purified on CH-Sepharose covalently coupled to the protein antigen used to raise the antibody. The monoclonal antibody recognising the STRADα protein has been described previously (Baas et al., 2003). The phospho-specific antibody recognizing MBP phosphorylated at Thr65 (T65-P) was raised in sheep against peptide GHHAARTTHYGSLPQ corresponding to residues 58-72 of bovine MBP in which the underlined residue is phosphothreonine. The antibody was affinity-purified on CH-Sepharose covalently coupled to the phosphorylated peptide and then passed through a column of CH-Sepharose coupled to the non-phosphorylated peptide. Antibody that did not bind to the latter column was selected. Monoclonal antibodies recognizing the GST and Flag epitope tags were obtained from Sigma, the monoclonal antibody recognizing the Myc epitope tag was purchased from Roche, and secondary antibodies coupled to horseradish peroxidase used for immunoblotting were obtained from Pierce. Preimmune IgG used in control immunoprecipitation experiments was obtained from preimmune serum employing Protein G-Sepharose.

General methods and buffers. Restriction enzyme digests, DNA ligations, and other recombinant DNA procedures were performed using standard protocols. DNA constructs used for transfection were purified from E. coli DH5α using Qiagen plasmid Mega kit according to the manufacturer's protocol. All DNA constructs were verified by DNA sequencing, which was performed by The Sequencing Service, School of Life Sciences, University of Dundee, Scotland, UK, using DYEnamic ET terminator chemistry (Amersham Biosciences) on Applied Biosystems automated DNA sequencers. Lysis Buffer contained 50 mM Tris/HCl pH 7.5, 1 mM EGTA, 1 mM EDTA, 1% (w/v) Triton-X 100, 1 mM sodium orthovanadate, 50 mM sodium fluoride, 5 mM sodium pyrophosphate, 0.27 M sucrose, 0.1% (v/v) 2-mercaptoethanol and ‘complete’ proteinase inhibitor cocktail (one tablet/50 ml). Buffer A contained 50 mM Tris/HCl pH 7.5, 0.1 mM EGTA, and 0.1% (v/v) 2-mercaptoethanol. SDS sample Buffer contained 50 mM Tris/HCl pH 6.8, 2% (w/v) SDS, 10% (v/v) glycerol, 0.005% (w/v) bromophenol blue, and 1% (v/v) 2-mercaptoethanol.

DNA constructs. Constructs expressing the full length wild type and kinase dead mouse LKB1 have been described previously (Sapkota et al., 2002a; Sapkota et al., 2002b; Sapkota et al., 2001). In order to generate the N-terminal Myc-tagged MO25α cDNA, a PCR was performed with primers: 5′-ggatccgccaccatggagcagaagctgatotctgaagaggacttgccgttcccgtttgggaagtctcacaaa t-3′ and 5′-ggatccttaagcttcttgctgagctggtctcttc-3′ using an IMAGE Consortium EST clone (IMAGE clone 4822388, NCBI Acc. BG719459) as a template. The resulting PCR product was ligated into pCR2.1-TOPO vector (Invitrogen), and subcloned as a BamH1-BamH1 fragment into the pEBG-2T (Sanchez et al., 1994), pCMV5 (Anderson et al., 1989) and pGEX-6T (Amersham) vectors. To generate the N-terminal Myc-tagged MO25β cDNA, a PCR was performed with primers 5′-caccggatccgccaccatggagcagaagctgatctctgaagaggacttgcctttgtttagtaaatcacacaa aaatcc-3′ and 5′-ggatcctcaaggggccgttttcttcaagtctcgg-3′ using an IMAGE Consortium EST clone (IMAGE 1958217, NCBI Acc. AI252223) as a template. The resulting PCR product was ligated into pENTR/D-TOPO Gateway intermediate vector (Invitrogen), and converted into Gateway modified pEBG-2T, pCMV5 and pGEX-6T expression vectors. It should be noted that the human MO25, IMAGE clone we obtained encodes for an Ala instead of a Val at position 153, compared to the submitted sequence (NCBI accession number Q9H9S4). Sequence analysis of the nucleotide sequence of several independent MO25β sequences listed Table 1, indicated that all of these possess an Ala at position 153. Expression constructs for Flag-tagged STRADα were PCR amplified with primers 5′-ggatccgccaccatggactacaaggacgacgatgacaagtcatttcttgtaagtaaaccagagcgaatc-3′ and 5′-ggatcctcagaactcccaatcgtccacctccagct-3 using a human STRADα cDNA as a template (Baas et al., 2003). The PCR product was ligated into pCR2.1-TOPO vector and subcloned as a BamH1-Not1 fragment into pEBG-2T or as a BamH1-Xba1 insert into pCMV5.

In order to clone STRADβ, two IMAGE EST clones (5301936 and 5501540), which together encompass the entire coding sequence were used as templates to generate full length flag-tagged STRADβ cDNA by PCR using primers 5′-ggatccgccaccatggactacaaggacgacgatgacaagtctcttttggattgcttctgcacttcaag-3′ and 5′-ggatccctagaattcccagtatgagtctttttcatc-3′. The PCR product was ligated into pCR2.1-TOPO vector and subcloned as a BamH1-BamH1 fragment into the pEBG-2T and pCMV5 vectors. The catalytic fragment of mouse LKB1 (residues 44-343) possessing an N-terminal Flag epitope tag was PCR amplified from a mouse LKB1 cDNA (Sapkota et al., 2001) using the primers: 5′-actagtgccaccatggactacaaggacgacgatgacaagaagctcatcggcaagtacctgatgggg-3′ and 5′-actagttcagtcctccaggtagggcactacagtcat-3′ and subcloned into pCMV5 vector as an SpeI-SpeI fragment. The construct encoding for the expression of GFP tagged human LKB1 with no other epitope tag was described previously (Boudeau et al., 2003b). Site directed mutagenesis was performed employing standard procedures using the Quickchange mutagenesis kit (Stratagene). Deletion mutations were generated by introducing STOP codons at the indicated positions.

Cell culture conditions and cell lysis. The generation of HeLa cells stably expressing wild type or kinase dead LKB1 has been described previously (Sapkota et al., 2002b). The cells were cultured in Minimum Essential Medium Eagle supplemented with 10% (v/v) tetracyclin-free FBS; in the presence of 1× non-essential amino acid solution, 1× Penicillin/Streptomycin solution (Invitrogen), 100 μg/ml zeocin and 5 μg/ml blasticidin (Invitrogen). Human embryonic kidney (HEK) 293, embryonic fibroblast Rat-2 cells, and HeLa cells were maintained in Dulbecco's modified Eagle's medium supplemented with 10% (v/v) FBS. For all experiments, cells were cultured on a 10-cm diameter dish and lysed in 0.5 to 1 ml of ice-cold Lysis Buffer. Lysates were clarified by centrifugation at 4° C. for 10 min at 14 000 g.

Immunoblotting. The amounts of proteins indicated in the figure legends were subjected to SDS-polyacrylamide gel electrophoresis and transferred to nitrocellulose. The membranes were blocked for 1 hour in 50 mM Tris/HCl (pH 7.5), 0.15 M NaCl, 0.5% (v/v) Tween containing 10% (w/v) skimmed milk powder for 1 h. The membranes were then incubated in the same buffer containing 1 μg/ml antibody (LKB1, MO25α and MO25β antibodies) or 0.1 μg/ml (STRADα antibody) for 8 h at 4° C. Immunoblotting with antibody T65-P was carried out as described above, except that the non-phosphorylated peptide corresponding to the antigen used to raise the antibody was included at a concentration of 10 μg/ml. Immunoblotting with the GST, Flag, and Myc antibodies was carried out as above with 0.5 μg/ml antibody except that skimmed milk powder was replaced with 5% (w/v) BSA. Detection was performed using the appropriate horseradish peroxidase-conjugated secondary antibodies and the enhanced chemiluminescence reagent (Amersham Pharmacia Biotech).

Immunoprecipitation of endogenous LKB1 and MO25α. The LKB1 and MO25α antibodies were covalently coupled to protein G-Sepharose in a ratio of 1 mg of antibody to 1 ml of resin using a dimethyl pimelimidate cross-linking procedure (Harlow and Lane, 1988). Clarified lysates of 293 and Rat-2 cells generated as described above except that 2-mercaptoethanol was omitted from the lysis buffer, containing 2 mg of total cell protein were incubated at 4° C. for 1 h on a vibrating platform with 10 μl of the LKB1-protein G-Sepharose conjugate or 15 μl of the MO25α-protein G-Sepharose conjugate. The immunoprecipitates were washed twice with 1 ml of Lysis Buffer (not containing 2-mercaptoethanol) containing 150 mM NaCl and twice with 1 ml of Buffer A (not containing 2-mercaptoethanol). The beads were then resuspended in a volume of 20 μl of Buffer A to which 5 μl of SDS sample buffer lacking 2-mercaptoethanol was added. The samples were subjected to electrophoresis and then immunoblotted for LKB1, STRADα and MO25α as described above.

Expression of GST-fusion proteins in 293 cells and affinity purification. 10-cm diameter dishes of 293 cells were transiently transfected with 3 to 10 μg of the pEBG-2T constructs using a modified calcium phosphate method (Alessi et al., 1996). 36 h post-transfection, the cells were lysed and the clarified lysates were incubated for 1 h on a rotating platform with glutathione-Sepharose (25 μl/dish of lysate) previously equilibrated in lysis Buffer. The beads were washed 4 times with lysis Buffer containing 150 mM NaCl and twice with Buffer A. For immunoblotting analysis, the beads were then resuspended in SDS sample Buffer after this step to give a final volume of slurry of 75 μl, and the samples immunoblotted as described above. For protein kinase assays and gel electrophoresis, the beads were washed a further twice with Buffer A containing 0.27 M sucrose and the proteins were eluted from the resin by incubation with the same buffer containing 20 mM of glutathione. The beads were then removed by filtration through a 0.44-μm filter and the eluate divided into aliquots and stored at −80° C.

Expression and purification of GST-MO25 isoforms in E. coli. The pGEX-6T constructs encoding N-terminally GST tagged MO25α or GST tagged MO25β were transformed into E. coli BL21 cells and a 0.5-liter culture was grown at 37° C. in Luria-Bertani broth until the absorbance at 600 nm was ˜0.7. Isopropyl-β-D-thiogalactopyranoside was then added at a concentration of 250 μM to induce protein expression, and the cells were cultured for a further 16 h at 26° C. The cells were harvested by centrifugation for 30 min at 5 000 g and resuspended in 25 ml of Lysis Buffer. Lysis was completed by one round of freeze/thawing and ten 15-sec cycles of sonication. The lysate was centrifuged at 4° C. for 30 min at 20 000 g, the supernatant filtered through a 0.44 μm filter and incubated for 1 h on a rotating platform with 1 ml of glutathione-Sepharose previously equilibrated in lysis Buffer. The beads were washed 4 times with lysis Buffer containing 150 mM NaCl, twice with Buffer A and twice with Buffer A containing 0.27 M sucrose. In order to elute MO25 isoforms from the resin, 15 μg of GST tagged PreScission protease was added which cleaves the N-terminal GST tag on the GST-fusion proteins. The resin was incubated with the protease for 16 h at 4° C. on a rotating platform and the eluted MO25 proteins collected by filtration through a 0.44-μm filter. The eluted MO25 isoforms were further incubated with 0.25 ml of glutathione-Sepharose for 30 min at 4° C. to remove any traces of uncleaved GST-fusion protein and GST-PreScission protease. 1 mg of homogenous MO25 was typically isolated and after snap freezing in liquid nitrogen stored in aliquots at −80° C.

Purification of Flag-LKB1 and mass spectrometry analysis. Flag-LKB1 was immuno-purified from HeLa cells stably expressing wild type LKB1 or kinase dead LKB1, or from control HeLa cells using anti-Flag M2-affinity agarose gel as described previously (Boudeau et al., 2003a). For each purification, hundred 10 cm diameter dishes of each HeLa cell line was employed and yielded a 0.4 ml of eluted affinity purified LKB1 solution. 40 μl of each sample (FIG. 1A lane 1-3) were electrophoresed on a precast 4-12% SDS-polyacrylamide gel, and the gel stained with colloidal blue and photographed. For the concentrated sample (FIG. 1A, lane 4), 250 μl of the KD-LKB1 preparation was methanol precipitated, resuspended in 1× SDS Sample Buffer and analysed. The bands indicated in FIG. 1A were excised, washed and digested with trypsin as described previously (Woods et al., 2001). Tryptic peptides were analysed by MALDI-TOF mass spectrometry, using α-cyanocinnamic acid as the matrix, on a PerSeptive Biosystems Elite STR mass spectrometer. Spectra were acquired in the reflectron mode, internally calibrated and the tryptic peptide masses were scanned against NCBInr and SwissProt databases using the MS-FIT program of Protein Prospector (UCSF, CA) run on a local server. The identity of STRADα and MO25α was also confirmed by LC-MS/MS sequence analysis on a Q-TOF2 mass spectrometer as described previously (Way et al., 2002).

Affinity purification of MO25α with peptides. Streptavidin-Sepharose previously equilibrated in PBS was incubated with each biotinylated peptide in a ratio of 1 μg of peptide to 1 μl of resin on a vibrating platform for 30 min, and the beads were then washed twice with PBS to remove any unconjugated peptide. The indicated cell lysates (0.5 mg) were incubated with Streptavidin-Sepharose peptide conjugate (5 μl) for 1 h at 4° C. on a vibrating platform. The beads were washed twice with 1 ml of Lysis Buffer containing 150 mM NaCl and twice with 1 ml of Buffer A, and then resuspended in a volume of 20 μl of Buffer A to which 5 μl of SDS sample buffer was added. The samples were then subjected to electrophoresis and immunoblotted for MO25α as described above.

Biacore analysis. Binding was analysed in a BiaCore 3000 system (BiaCore AB, Stevenage, UK). The indicated biotinylated peptides were bound to a streptavidin-coated Sensor chip (SA) (20 response units, RU). Various concentrations of bacterially-expressed MO25α previously dialysed in buffer HBS-EP (10 mM HEPES pH 7.4, 0.15 M NaCl, 3 mM EDTA, 0.005% (v/v) polysorbate-20), were injected over the immobilised peptides at a flow rate of 30 μl/min. Interactions between each peptide and the MO25α protein were analysed over a range of MO25α concentrations of 6.25-3200 nM and steady state binding was determined at each concentration. Dissociation of MO25α from each peptide was monitored over a 1 min period. Regeneration of the Sensor chip surface between each injection was performed with 3 consecutive 5-μl injections of 10 mM NaOH, 1 M NaCl.

LKB1 autophosphorylation and phosphorylation of MBP. 0.5 μg of wild-type or catalytically inactive GST-LKB1 of the indicated complexes of LKB1 (FIG. 8) that had been affinity purified on glutathione-Sepharose from 293 cells, was incubated in a 30-μl reaction mixture containing 50 mM Tris-HCl (pH 7.5), 0.1% 2-mercaptoethanol, 0.1 mM EGTA, 10 mM manganese chloride, 0.5 μM microcystin-LR, and 100 μM [γ-³²P]ATP (1000 cpm/pmol) in the presence of 3.3 μg of bovine MBP. After incubation for 40 min at 30° C., the reactions were terminated by the addition of 8 μl of sample Buffer. 30 μl was electrophoresed on SDS-polyacrylamide gels, which were then dried and analysed by autoradiography to quantify LKB1 autophosphorylation, MBP and STRADα phosphorylation. 5 μl of each reaction was diluted in 45 μl of 1× SDS Sample Buffer. Of the resulting solution, 10 μl were used for immunoblotting with the anti-GST (recognises LKB1), anti-Flag (recognises STRADα and STRADβ) and anti-Myc (recognises MO25α and MO25β) antibodies. 30 μl of the sample was used for immunoblotting with the T65-P MBP antibody.

Localisation studies. HeLa cells were cultured to 50% confluence on glass coverslips (no. 1.5) on 10-cm diameter dishes and transfected with a total of 2 μg of a construct encoding wild-type GFP-LKB1 together with the indicated pCMV5 constructs using Effectene transfection reagent (Qiagen) according to the manufacturers protocol. A duplicate set of dishes was used for each condition. The cells were washed with PBS 20 h post-transfection, and were fixed for 10 min in freshly prepared 4% (v/v) paraformaldehyde in PHEM Buffer (60 mM Pipes, 25 mM Hepes, 10 mM EGTA and 2 mM magnesium sulphate, pH 7.0). The cells were then washed twice with PBS and permeablised with 1% (by vol) NP40 in PBS and blocked with 10% normal goat serum. The cells were immunolabelled with both the anti-MO25α antibody and anti-Flag antibody (to detect Flag-tagged STRADα), washed in PBS and counter stained with Texas Red anti-sheep IgG and Cy5 anti-mouse IgG antibodies. The cells were imaged using a Zeiss LSM 510 META confocal microscope. Each channel was scanned independently to avoid cross talk (Multi-Tracking).

Northern Blot Analysis. A cDNA corresponding to human MO25α (NCBI Acc, N. NM_{—}016289, nucleotide residues 1550 to 2500), was ³²P-labelled by random priming using a Hi Prime DNA labelling kit (Roche). This probe was used to screen a Multiple Tissue Northern blot (Clontech, PT1200-1) using Rapid-Hyb Buffer (Amersham Pharmacia) according to the protocol provided by the manufacturer. Following autoradiography, the probe was stripped and the blot was then re-hybridised as above with a β-actin probe provided by the manufacturer.

### REFERENCES

Alessi, D. R., Caudwell, F. B., Andjelkovic, M., Hemmings, B. A. and Cohen, P. (1996) Molecular basis for the substrate specificity of protein kinase B; comparison with MAPKAP kinase-1 and p70 S6 kinase. FEBS Lett, 399, 333-338.
Anderson, S., Davie, D. N., Dahlback, H., Jörnvall, H. and Russell, D. W. (1989) Cloning, structure, and expression of the mitochondrial cytochrome-P-450 sterol 26-hydroxylase, a bile-acid D biosynthetic enzyme. J. Biol. Chem., 264, 8222-8229.
Harlow, E. and Lane, D. (1988) Antibodies a Laboratory Manual. Cold Spring Harbor Laboratory Publications, New York.
Lizcano, J. M., Deak, M., Morrice, N. and Alessi, D. R. (2002) Identification of the residues of S6K, SGK and PKB that are phosphorylated by NEK6. Manuscript in Preparation.
Sanchez, I., Hughes, R. T., Mayer, B. J., Yee, K., Woodgett, J. R., Avruch, J., Kyriakis, J. M. and Zon, L. I. (1994) Role of SAPK/ERK kinase-1 in the stress-activated pathway regulating transcription factor c-Jun. Nature, 372, 794-798.
Way, G., Morrice, N., Smythe, C. and O'Sullivan, A. J. (2002) Purification and identification of secernin, a novel cytosolic protein that regulates exocytosis in mast cells. Mol Biol Cell, 13, 3344-3354.
Woods, Y. L., Rena, G., Morrice, N., Barthel, A., Becker, W., Guo, S., Unterman, T. G. and Cohen, P. (2001) The kinase DYRK1A phosphorylates the transcription factor FKHR at Ser329 in vitro, a novel in vivo phosphorylation site. Biochem J, 355, 597-607.
Baas, A. F., Boudeau, J., Sapkota, G. P., Smit, L., Medema, R., Morrice, N. A., Alessi, D. R. and Clevers, H. C. (2003) Activation of the tumour suppressor kinase LKB1 by the STE20-like Pseudokinase STRAD. EMBO J. 22(12), 3062-3072
Bardeesy, N., Sinha, M., Hezel, A. F., Signoretti, S., Hathaway, N. A., Sharpless, N. E., Loda, M., Carrasco, D. R. and DePinho, R. A. (2002) Loss of the Lkb1 tumour suppressor provokes intestinal polyposis but resistance to transformation. Nature, 419, 162-167.
Bidlingmaier, S., Weiss, E. L., Seidel, C., Drubin, D. G. and Snyder, M. (2001) The Cbk1p pathway is important for polarized cell growth and cell separation in Saccharomyces cerevisiae. Mol Cell Biol, 21, 2449-2462.
Boudeau, J., Deak, M., Lawlor, M. A., Morrice, N. A. and Alessi, D. R. (2003a) Heat-shock protein 90 and Cdc37 interact with LKB1 and regulate its stability. Biochem J, 370, 849-857.
Boudeau, J., Kieloch, A., Alessi, D. R., Stella, A., Guanti, G. and Resta, N. (2003b) Functional analysis of LKB1/STK11 mutants and two aberrant isoforms found in Peutz-Jeghers Syndrome patients. Hum Mutat, 21, 172.
Buchet-Poyau, K., Mehenni, H., Radhakrishna, U. and Antonarakis, S. E. (2002) Search for the second Peutz-Jeghers syndrome locus: exclusion of the STK13, PRKCG, KLK10, and PSCD2 genes on chromosome 19 and the STK11IP gene on chromosome 2. Cytogenet Genome Res, 97, 171-178.
Collins, S. P., Reoma, J. L., Gamm, D. M. and Uhler, M. D. (2000) LKB1, a novel serine/threonine protein kinase and potential tumour suppressor, is phosphorylated by cAMP-dependent protein kinase (PKA) and prenylated in vivo. Biochem J, 345 Pt 3, 673-680.
Dorland, S., Deegenaars, M. L. and Stillman, D. J. (2000) Roles for the Saccharomyces cerevisiae SDS3, CBK1 and HYM1 genes in transcriptional repression by SIN3. Genetics, 154, 573-586.
Hemminki, A. (1999) The molecular basis and clinical aspects of Peutz-Jeghers syndrome. Cell Mol Life Sci, 55, 735-750.
Hemminki, A., Markie, D., Tomlinson, I., Avizienyle, E., Roth, S., Loukola, A., Bignell, G., Warren, W., Aminoff, M., Hoglund, P., Jarvinen, H., Kristo, P., Pelin, K., Ridanpaa, M., Salovaara, R., Toro, T., Bodmer, W., Olschwang, S., Olsen, A. S., Stratton, M. R., de la Chapelle, A. and Aaltonen, L. A. (1998) A serine/threonine kinase gene defective in Peutz-Jeghers syndrome. Nature, 391, 184-187.
Jenne, D. E., Reimann, H., Nezu, J., Friedel, W., Loff, S., Jeschke, R., Muller, O., Back, W. and Zimmer, M. (1998) Peutz-Jeghers syndrome is caused by mutations in a novel serine threonine kinase. Nat Genet, 18, 38-43.
Jishage, K. I., Nezu, J. I., Kawase, Y., Iwata, T., Watanabe, M., Miyoshi, A., Ose, A., Habu, K., Kake, T., Kamada, N., Ueda, O., Kinoshita, M., Jenne, D. E., Shimane, M. and Suzuki, H. (2002) Role of Lkb1, the causative gene of Peutz-Jegher's syndrome, in embryogenesis and polyposis. Proc Natl Acad Sci USA, 99, 8903-8908.
Johnston, A. M., Naselli, G., Gonez, L. J., Martin, R. M., Harrison, L. C. and DeAizpurua, H. J. (2000) SPAK, a STE20/SPS1-related kinase that activates the p38 pathway. Oncogene, 19, 4290-4297.
Jorgensen, P., Nelson, B., Robinson, M. D., Chen, Y., Andrews, B., Tyers, M. and Boone, C. (2002) High-Resolution Genetic Mapping With Ordered Arrays of Saccharomyces cerevisiae Deletion Mutants. Genetics, 162, 1091-1099.
Karos, M. and Fischer, R. (1999) Molecular characterization of HymA, an evolutionarily highly conserved and highly expressed protein of Aspergillus nidulans. Mol Gen Genet, 260, 510-521.
Karuman, P., Gozani, O., Odze, R. D., Zhou, X. C., Zhu, H., Shaw, R., Brien, T. P., Bozzuto, C. D., Ooi, D., Cantley, L. C. and Yuan, J. (2001) The Peutz-Jegher gene product LKB1 is a mediator of p53-dependent cell death. Mol Cell, 7, 1307-1319.
Marignani, P. A., Kanai, F. and Carpenter, C. L. (2001) Lkb1 associates with brg1 and is necessary for brg1-induced growth arrest. J Biol Chem, 276, 32415-32418.
Martin, S. G. and St Johnston, D. (2003) A role for Drosophila LKB1 in anterior-posterior axis formation and epithelial polarity. Nature, 421, 379-384.
Miyamoto, H., Matsushiro, A. and Nozaki, M. (1993) Molecular cloning of a novel mRNA sequence expressed in cleavage stage mouse embryos. Mol Reprod Dev, 34, 1-7.
Miyoshi, H., Nakau, M., Ishikawa, T. O., Seldin, M. F., Oshima, M. and Taketo, M. M. (2002) Gastrointestinal hamartomatous polyposis in Lkb1 heterozygous knockout mice. Cancer Res, 62, 2261-2266.
Nakau, M., Miyoshi, H., Seldin, M. F., Imamura, M., Oshima, M. and Taketo, M. M. (2002) Hepatocellular carcinoma caused by loss of heterozygosity in lkb1 gene knockout mice. Cancer Res, 62, 4549-4553.
Nishigaki, K., Thompson, D., Yugawa, T., Rulli, K., Hanson, C., Cmarik, J., Gutkind, J. S., Teramoto, H. and Ruscetti, S. (2003) Identification and characterization of a novel Ste-20/GCK-related kinase, PAP kinase (PAPK). J Biol Chem.
Nozaki, M., Onishi, Y., Togashi, S. and Miyamoto, H. (1996) Molecular characterization of the Drosophila Mo25 gene, which is conserved among Drosophila, mouse, and yeast. DNA Cell Biol, 15, 505-509.
Resta, N., Stella, A., Susca, F. C., Di Giacomo, M., Forleo, G., Miccolis, I., Rossini, F. P., Genuardi, M., Piepoli, A., Grammatico, P. and Guanti, G. (2002) Two novel mutations and a new STK11/LKB1 gene isoform in Peutz-Jeghers patients. Hum Mutat, 20, 78-79.
Rossi, D. J., Ylikorkala, A., Korsisaari, N., Salovaara, R., Luukko, K., Launonen, V., Henkemeyer, M., Ristimaki, A., Aaltonen, L. A. and Makela, T. P. (2002) Induction of cyclooxygenase-2 in a mouse model of Peutz-Jeghers polyposis. Proc Natl Acad Sci USA, 99, 12327-12332.
Sanna, M. G., da Silva Correia, J., Luo, Y., Chuang, B., Paulson, L. M., Nguyen, B., Deveraux, Q. L. and Ulevitch, R. J. (2002) ILPIP, a novel anti-apoptotic protein that enhances XIAP-mediated activation of JNK1 and protection against apoptosis. J Biol Chem, 277, 30454-30462.
Sapkota, G. P., Boudeau, J., Deak, M., Kieloch, A., Morrice, N. and Alessi, D. R. (2002a) Identification and characterization of four novel phosphorylation sites (Ser31, Ser325, Thr336 and Thr366) on LKB1/STK11, the protein kinase mutated in Peutz-Jeghers cancer syndrome. Biochem J, 362, 481-490.
Sapkota, G. P., Deak, M., Kieloch, A., Morrice, N., Goodarzi, A. A., Smythe, C., Shiloh, Y., Lees-Miller, S. P. and Alessi, D. R. (2002b) Ionizing radiation induces ataxia telangiectasia mutated kinase (ATM)-mediated phosphorylation of LKB1/STK11 at Thr-366. Biochem J, 368, 507-516.
Sapkota, G. P., Kieloch, A., Lizcano, J. M., Lain, S., Arthur, J. S., Williams, M. R., Morrice, N., Deak, M. and Alessi, D. R. (2001) Phosphorylation of the protein kinase mutated in Peutz-Jeghers Cancer Syndrome, LKB1/STK11, at Ser431 by p90RSK and cAMP-dependent protein kinase, but not its farnesylation at Cys433, is essential for LKB1 to suppress cell growth. J Biol Chem, 276, 19469-19482.
Smith, C. M., Radzio-Andzelm, E., Madhusudan, Akamine, P. and Taylor, S. S. (1999) The catalytic subunit of cAMP-dependent protein kinase: prototype for an extended network of communication. Prog Biophys Mol Biol, 71, 313-341.
Smith, D. P., Rayter, S. I., Niederlander, C., Spicer, J., Jones, C. M. and Ashworth, A. (2001) LIP1, a cytoplasmic protein functionally linked to the Peutz-Jeghers syndrome kinase LKB1. Hum Mol Genet, 10, 2869-2877.
Songyang, Z., Fanning, A. S., Fu, C., Xu, J., Marfatia, S. M., Chishti, A. H., Crompton, A., Chan, A. C., Anderson, J. M. and Cantley, L. C. (1997) Recognition of unique carboxyl-terminal motifs by distinct PDZ domains. Science, 275, 73-77.
Tamari, M., Daigo, Y. and Nakamura, Y. (1999) Isolation and characterization of a novel serine threonine kinase gene on chromosome 3p22-21.3. J Hum Genet, 44, 116-120.
Tiainen, M., Vaahtomeri, K., Ylikorkala, A. and Makela, T. P. (2002) Growth arrest by the LKB1 tumor suppressor: induction of p21(WAF1/CIP1). Hum Mol Genet, 11, 1497-1504.
Tiainen, M., Ylikorkala, A. and Makela, T. P. (1999) Growth suppression by Lkb1 is mediated by a G(1) cell cycle arrest. Proc Natl Acad Sci USA, 96, 9248-9251.
Watts, J. L., Morton, D. G., Bestman, J. and Kemphues, K. J. (2000) The C. elegans par-4 gene encodes a putative serine-threonine kinase required for establishing embryonic asymmetry. Development, 127, 1467-1475.
Ylikorkala, A., Rossi, D. J., Korsisaari, N., Luukko, K., Alitalo, K., Henkemeyer, M. and Makela, T. P. (2001) Vascular abnormalities and deregulation of VEGF in Lkb1-deficient mice. Science, 293, 1323-1326.
Yoo, L. I., Chung, D. C. and Yuan, J. (2002) LKB1 A master tumour suppressor of the small intestine and beyond. Nat Rev Cancer, 2, 529-535.

### EXAMPLE 2

### Complexes Between the LKB1 Tumour Suppressor, Stradα/β and Mo25α/β are Upstream Kinases in the AMP-Activated Protein Kinase Cascade

The AMP-activated protein kinase (AMPK) cascade is a sensor of cellular energy charge that acts as a “metabolic master switch” and inhibits cell proliferation. Activation requires phosphorylation within the activation loop (Thr-172) by upstream kinases (AMPKKs) that have not been identified. Recently we identified three related protein kinases acting upstream of the yeast homologue of AMPK, which do not have obvious mammalian homologues. However, a closely related mammalian kinase is LKB1, for which no physiological substrates have been identified. LKB1 acts as a tumour suppressor and is mutated in human Peutz-Jeghers syndrome. We show in Example 1 that it exists as a complex with two accessory subunits, STRADα/β and MO25α/β.

We report that: (i) two AMPKK activities purified from rat liver contain LKB1, STRADα and MO25α, and can be immunoprecipitated using anti-LKB1 antibodies; (ii) both endogenous and recombinant complexes of LKB1, STRADα/β and MO25α/β activate AMPK via phosphorylation of Thr-172, (iii) catalytically active LKB1 and STRADα or STRADβ and MO25α or MO25β are required for full activity; (iv) the drugs AICA riboside and phenformin do not activate AMPK in HeLa cells (which lack LKB1), but this can be restored by stably expressing wild type, but not catalytically inactive, LKB1 in these cells.

These results provide the first description of a physiological substrate for the LKB1 tumour suppressor and suggest that it functions as an upstream regulator of AMPK. Our findings indicate that the formation of tumours in Peutz-Jeghers syndrome could result from deficient activation of AMPK as a consequence of LKB1 inactivation.

Results

Resolution of Two AMPKKs from Rat Liver Extracts

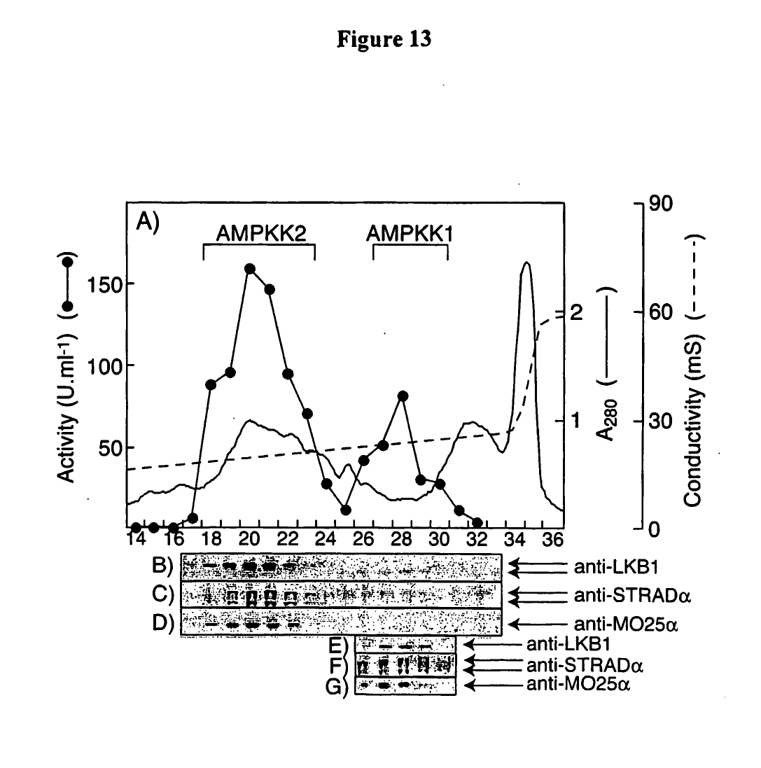

While experimenting with different conditions to optimise recovery at the Q-Sepharose step of our previous purification protocol for AMPKK [14), we found that we were able to resolve two peaks of activity (FIG. 13A). Because the second peak appears to correspond to the AMPKK originally purified [14], we refer to it as AMPKK1, with the first peak eluting from the column termed AMPKK2, The AMPKK assays in FIG. 13A were conducted using as a substrate a bacterially expressed glutathione-S-transferase (GST) fusion of the catalytic domain of rat AMPKα1. This is more convenient than the native rat liver AMPK heterotrimer used previously (14] because the AMPKα1 catalytic domain is already completely dephosphorylated and therefore inactive when expressed in bacteria. It is also possible to carry out the assays with the substrate bound to glutathione-Sepharose beads, allowing any endogenous AMPK in the AMPKK fraction that might otherwise interfere to be washed away. On size exclusion chromatography on Sephacryl S-200, AMPKK1 and AMPKK2 eluted as proteins of large but distinct size. By comparison with standards, the Stokes radii for AMPKK1 and AMPKK2 were estimated as 5.2 and 5.7 nm respectively. Ca2+ and calmodulin stimulated neither AMPKK1 nor AMPKK2, and neither were immunoprecipitated using an antibody against CAMKK (not shown).

AMPKK1 and AMPKK2 Both Contain LKB1, STRADα and MO25α

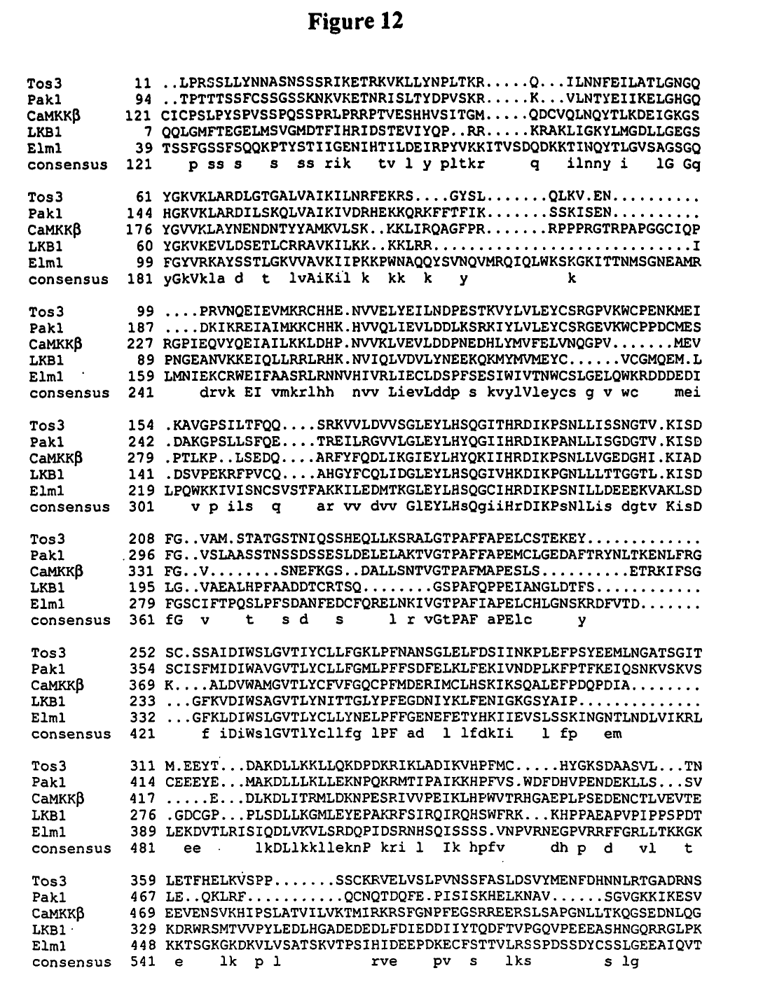

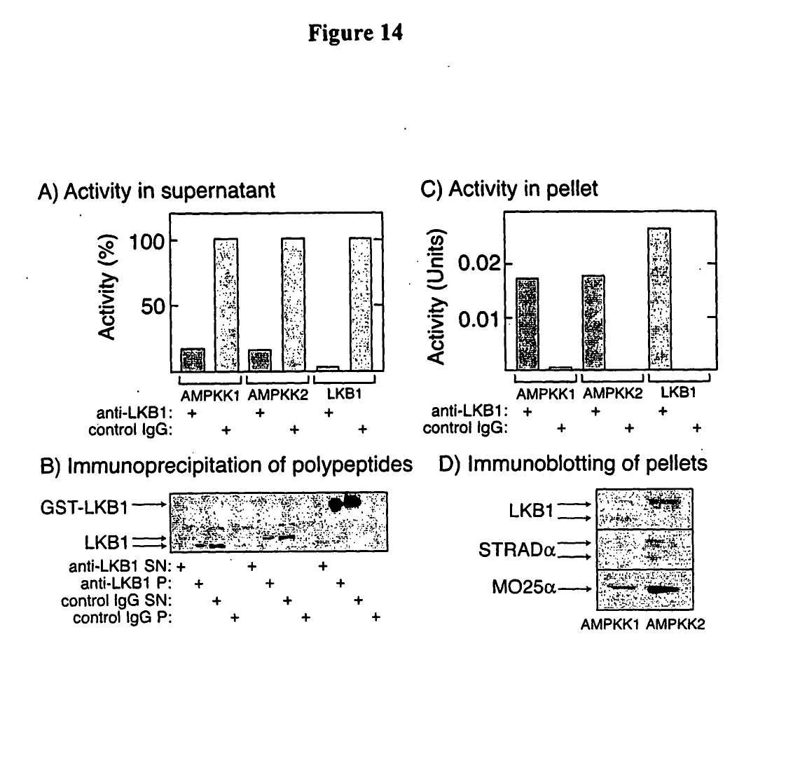

To examine whether either of these two fractions might contain LKB1, we probed blots of fractions across the Q-Sepharose chromatography using antibodies against LKB1, STRADα and MO25α (FIG. 13B-D). This revealed that the activity of AMPKK2 correlated with the presence of the ˜50 kDa polypeptide of LKB1 (≈50 kDa) as well as those of STRADα (≈45/48 kDa) and Mo25α (≈40 kDa) in fractions 18-22^{˜}. STRADα was detected as a doublet as reported previously [30]; the reason for this behaviour is not known. We did not obtain any signal of the correct molecular mass in these fractions using anti-MO25β antibody (not shown), consistent with our observations that the MO25β isoform is not expressed in mouse liver (Example 1). We also obtained a faint signal for LKB1 and STRADα in fractions 27-29 that contained AMPKK1, but at this loading MO25α was below the limit of detection in these fractions. However, the presence of LKB1, STRADα and MO25α in AMPKK1 was confirmed by analysing a higher loading of fractions 26-30 (FIG. 12E-G). An interesting finding was that the LKB1 polypeptide migrated with a slightly faster mobility in AMPKK1 compared with AMPKK2: this is evident by inspection of FIG. 13B (see also FIGS. 14B and 14D).

AMPKK Activity can be Immunoprecipitated from AMPKK1 and AMPKK2

Using anti-LKB1 antibody but not a pre-immune control IgG, we were able to immunoprecipitate AMPKK activity from both AMPKK1 and AMPKK2. FIG. 14A shows results of an experiment where the amount of kinase kinases was limiting and the antibody was in excess, and shows that we were able to remove>80% of the activity from the peak fractions containing AMPKK1 and AMPKK2 by immunoprecipitating with anti-LKB1 antibody, while no activity was removed using a pre-immune control immunoglobulin. We could remove>95% of the AMPKK activity of a recombinant GST-tagged LKB1-STRADα-MO25α complex (see next section) under the same conditions. The small amount of activity remaining in the supernatants of the AMKK1 and AMPKK2 fractions could be accounted for by the fact that the immunoprecipitation was not quantitative in these cases, with about 20% of the LKB1 polypeptide remaining in the supernatant (FIG. 14B).

Due to the small amount of AMPKK1 and AMPKK2 used in this experiment, it proved difficult to analyse the pellets for AMPKK activity and the presence of the other polypeptides. We therefore repeated the experiment with more kinase kinase and less antibody (the amount of antibody was now limiting) and analysed the pellets only. This showed that we could recover almost as much AMPKK activity in the pellet from the peak fractions containing AMPKK1 and AMPKK2 as we could from the recombinant LKB1-STRADα-MO25α complex, with no activity being recovered using pre-immune control immunoglobulin (FIG. 14C). Western blotting of the AMPKK1 and AMPKK2 pellets showed that they contained LKB1, STRADα and MO25α, although the STRADα doublet was very faint in the AMPKK1 pellet (FIG. 14D).

Recombinant LKB1-STRAD-MO25 Complexes Activate AMPKα1 Catalytic Domain in Cell-Free Assays

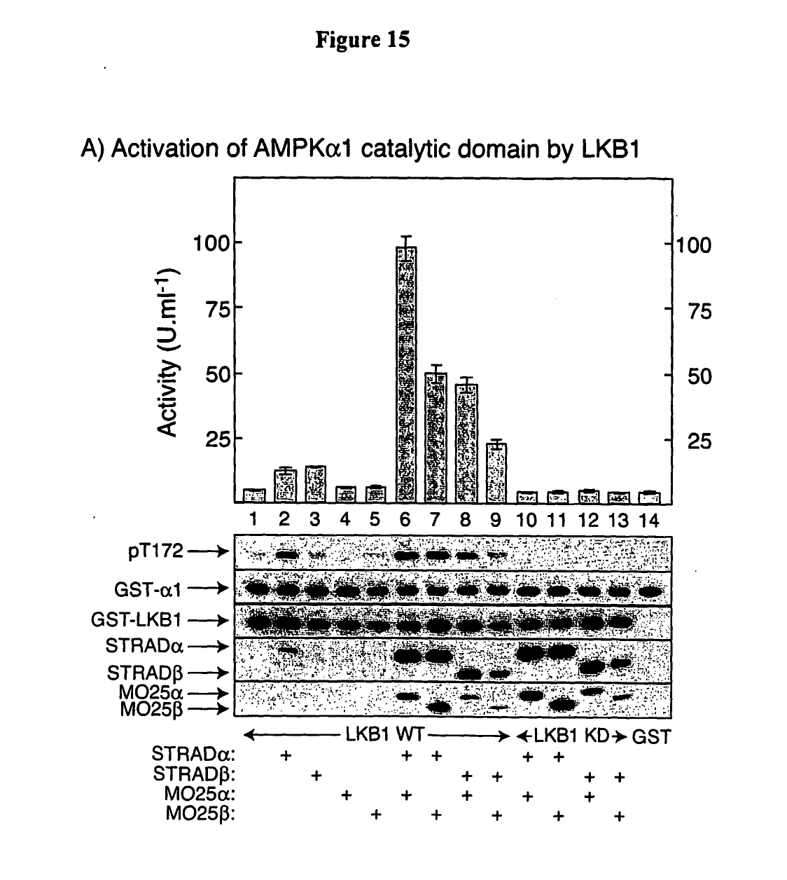

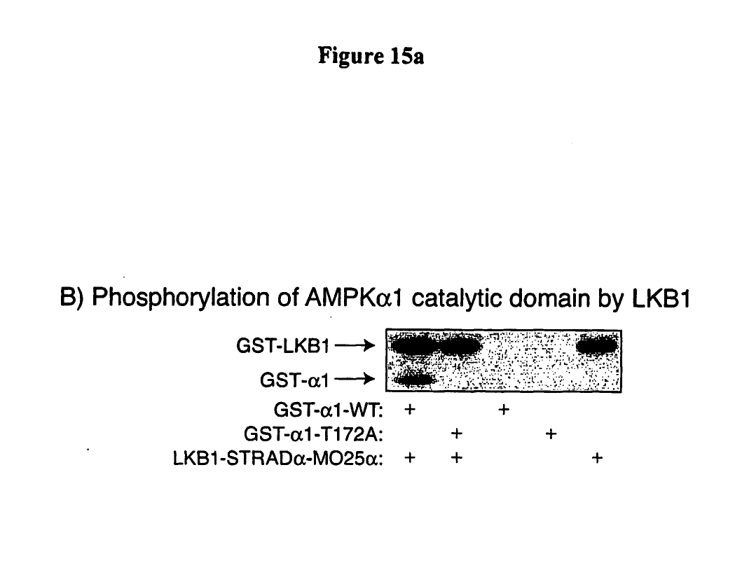

To examine whether LKB1 activated AMPK on its own or whether the accessory subunits STRADα/β and Mo25α/β were required, we expressed GST-tagged LKB1, FLAG-tagged STRADα/β and myc-tagged Mo25α/β in various combinations in HEK-293T cells, and purified the complexes on glutathione-Sepharose. We also used a GST-tagged kinase-inactive mutant of LKB1 (D194A), and a plasmid expressing GST alone, as controls. The purified complexes were incubated with the AMPKα1 catalytic domain in the presence of MgATP, and activation of the catalytic domain as well as phosphorylation of Thr172 (using a phosphospecific anti-pT172 antibody) was measured. FIG. 15A shows that LKB1 alone did not significantly increase the activity, or phosphorylation of Thr172, of the AMPKα1 catalytic domain above the basal activity observed in the presence of GST alone (compare lanes 1 and 14). The same result was obtained with LKB1 that had been co-expressed with MO25α or MO25β (lanes 4 and 5), which was expected as these proteins do not interact with LKB1 in the absence of STRADα/β (Example 1). An LKB1-STRADα complex did give a small but significant activation, and Thr172 phosphorylation, of the AMPKα1 catalytic domain above the basal value (compare lanes 1 and 2). However to produce a large activation and phosphorylation of the AMPKα1 catalytic domain, a heterotrimeric complex containing LKB1, STRADα or STRADβ, and MO25α or MO25β was required (lanes 6 to 9). With the heterotrimeric complexes the degree of activation was in the order LKB1-STRADα-MO25α>LKB1-STRADα-MO25β=LKB1-STRADβ-MO25α>LKB1-STRADβ-MO25β. Importantly, the ability of LKB1-STRAD-MO25 complexes to activate AMPKα1 was entirely dependent on LKB1 catalytic activity, because complexes of a catalytically inactive mutant of LKB1 D194A) with the various combinations of STRADα/β and MO25α/β lanes 10-13), were unable to activate or phosphorylate AMPKα1. The degree of activation obtained with these various complexes of wild type LKB1 correlated well with the phosphorylation of Thr-172. Probing with in antibody against the whole AMPKα1 catalytic domain (labelled GST-αα1-KD in FIG. 15A) confirmed that the loading was uniform in these samples. The bottom three panels in FIG. 15A confirm that the relevant STRAD and MO25 subunit co-precipitated with LKB1 when DNAs encoding these subunits had been co-transfected. When STRADα was co-expressed with LKB1 in the absence of an MO25 subunit, the amount of STRADα subunit co-precipitated with LKB1 was reduced (lanes 2 and 3). This is consistent with previous findings that the presence of an MO25 subunit stabilizes LKB1-STRAD complexes (Example 1).

FIG. 15B provides evidence that Thr-172 was the only site on the AMPKα1 catalytic domain phosphorylated by the LKB1-STRADα-MO25α complex. When the two proteins were incubated together in the presence of [γ-³²P]ATP, the wild type AMPKα1 catalytic domain became ³²P-labelled, but a Thr172→Ala mutant of the AMPKα1 catalytic domain did not.

AMPKK1, AMPKK2 and Recombinant LKB1-STRAD-MO25 Complexes Also Activate Hterotrimeric AMPK Complexes

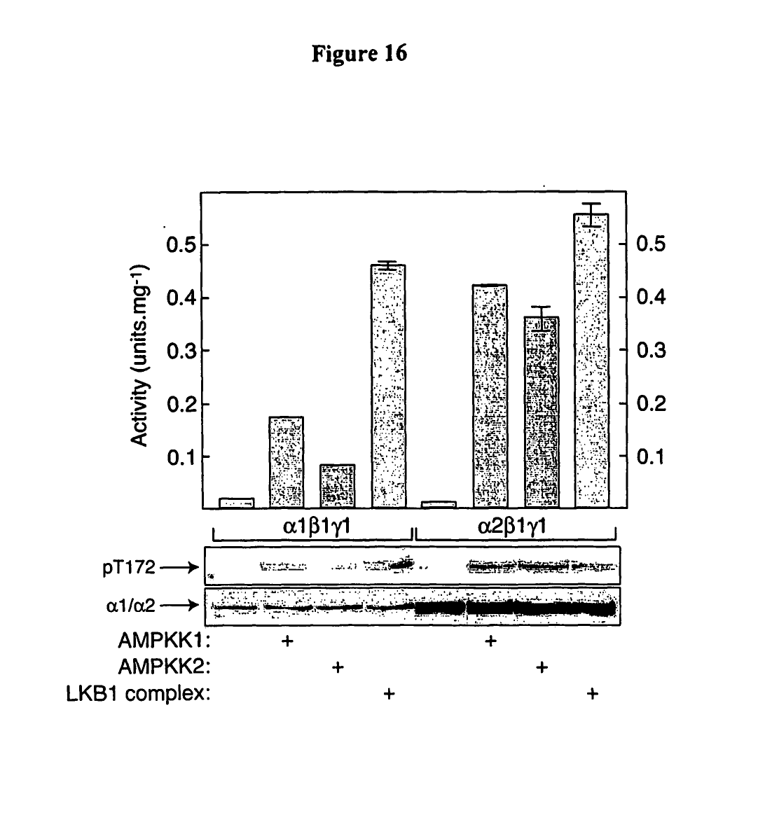

Although most of the AMPKK assays in this study were conducted using the AMPKα1 catalytic domain as substrate, FIG. 16 shows that AMPKK1, AMPKK2 and the recombinant GST-LKB1:STRADα:MO25α complex also activated heterotrimeric AMPK complexes. Plasmids encoding the α1, β1 and γ1 or α2, β1 and γ1 subunits of AMPK were co-expressed in CCL13 cells and purified by means of a myc tag on the α subunits. The recombinant AMPK complexes bound to protein G-Sepharose beads were incubated with MgATP and AMPKK1, AMPKK2 or GST-LKB1:STRADα:MO25α, the upstream kinase washed away and the activity of the AMPK complexes determined. FIG. 16 shows that both the α1β1γ1 and α2β1γ1 complexes were activated by all three upstream kinase preparations, and that this correlated with the phosphorylation of Thr-172 on the α subunits, shown using the anti-pT172 antibody (which recognises phosphorylated Thr-172 on both α1 and α2). The blot using anti-α1 and -α2 antibodies confirms uniform loading (note that a stronger signal is always obtained with the anti-α2 than the anti-α1 antibody, even when the protein loading is the same). Although the activity of AMPKK2 used was 3.5 times higher than that of AMPKK1 and only 30% lower than that of GST-LKB1-STRADα-MO25α in terms of its ability to activate the AMPKα1 catalytic domain, AMPKK2 appeared to be much less effective at activating the α1β1γ1 complex. These differences were less obvious using the α2β1γ1 complex as the substrate.

Endogenous LKB1 that Activates AMPK can be Immunoprecipitated from 293 Cells but not HeLa Cells

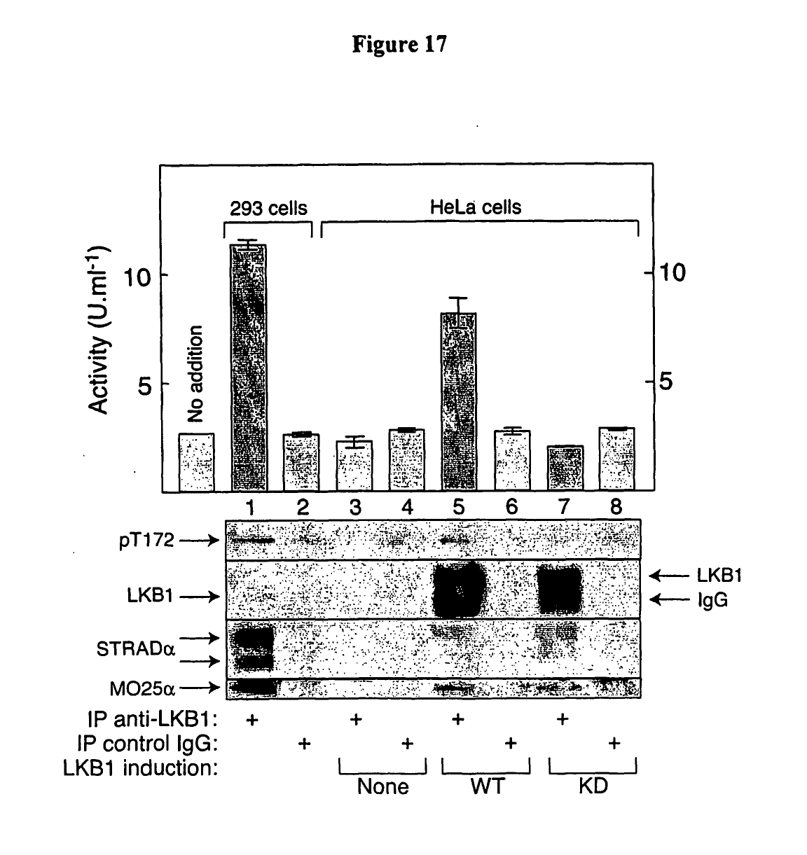

FIG. 17 shows that an activity that activated the AMPKα1 catalytic domain and phosphorylated Thr-172 could be immunoprecipitated from untransfected HEK-293T cell extract using anti-LKB1 antibody (lane 1), but not a pre-immune control immunoglobulin (lane 2). As reported previously [30, Example 1), immunoprecipitation of endogenous LKB1 resulted in the co-precipitation of STRADα and MO25α (lane 1). As a further control, we employed normal HeLa cells as an LKB1 null cell line, as it is known that LKB1 is not expressed in these cells [23]. Consistent with this, no LKB1, STRADα and MO25α subunits or AMPKK activity was immunoprecipitated from the same amount of HeLa cell extract protein using anti-LKB1 antibody (lane 3). However, AMPKK activity and Thr-172 phosphorylation, as well as detectable STRADα and MO25α subunits, were recovered following immunoprecipitation of LKB1 from HeLa cells [34], that stably express wild type LKB1 [32) (lane 5). In cells expressing a catalytically-inactive mutant of LKB1, the LKB1, STRADα and MO25α polypeptides were recovered by immunoprecipitation using anti-LKB1 antibody, but no AMPKK activity (lane 7). Although the LKB1 polypeptide was over-expressed to a large extent in the HeLa cells compared to the endogenous levels observed in 293 cells (FIG. 14, compare lanes 1 and 5), it is clear that the amount of STRADα and MO25α in the cells is limiting in these cells. There was a lower amount of AMPKK activity and Thr-172 phosphorylation, as well of co-precipitating STRADα and MO25α, in HeLa cells expressing LKB1 than in 293 cells, even though LKB1 itself was over-expressed. The AMPKK activity in the immunoprecipitates from 293 cells and HeLa cells expressing wild type LKB1 correlated with the levels of STRADα and MO25α in the complex rather than with the levels of LKB1. This further emphasises the essential nature of the STRAD and MO25 subunits in enhancing the AMPKK activity of LKB1.

Expression of LKB1 Restores Activation of AMPK in HeLa Cells

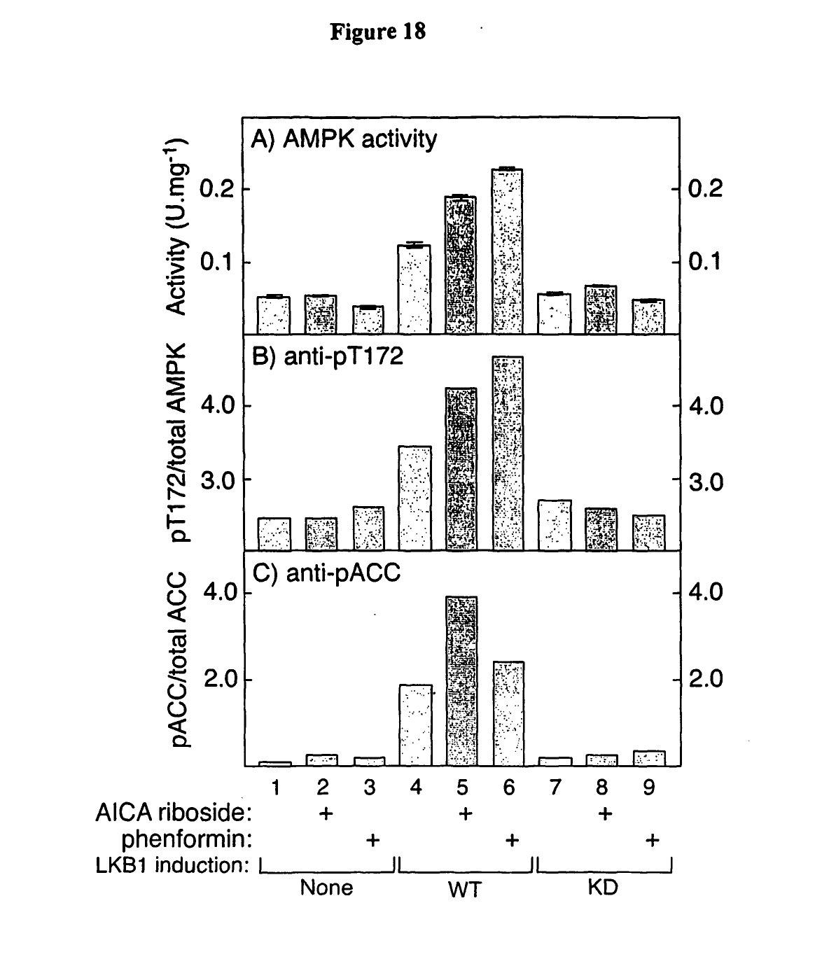

We have previously found (unpublished results) that, although AMPK is expressed in HeLa cells, it is not activated by the drugs 5-aminoimidazole-4-carboxamide (AICA) riboside and metformin, which readily activate the kinase in other cell types [33-34]. As HeLa cells do not express LKB1 [23] this could provide a potential explanation for this anomaly. To examine whether expression of recombinant LKB1 might restore the ability of HeLa cells to respond to these drugs, we used the HeLa cell that stably express wild type LKB1, or the catalytically inactive mutant [32]), as well as a control HeLa cell line not expressing LKB1. In these experiments we used phenformin, which is a close relative of metformin that we have found to activate AMPK more rapidly and more potently than metformin in other cell types. FIG. 18A shows that neither AICA riboside nor phenformin activated AMPK in control HeLa cells. However, in cells expressing wild type LKB1, but not the kinase-inactive mutant, both AICA riboside and phenformin caused a robust activation of AMPK. This correlated with the phosphorylation of Thr-172 on AMPKα, shown by probing with the anti-pT172 antibody (FIG. 18B), and also with the phosphorylation of a downstream target of AMPK, acetyl-CoA carboxylase (ACC), shown by probing with a phosphospecific antibody against Ser-79, the primary AMPK site on that protein (anti-pACC, FIG. 18C). Interestingly, stable expression of wild type LKB1 in the HeLa cells caused a small but reproducible degree of up-regulation of expression of AMPKα1 and a marked down-regulation of expression of ACC (not shown). Because the expression of these proteins was not uniform in these cells, in order to accurately quantitate their phosphorylation status we simultaneously probed single blots of lysates using either anti-pT172 and anti-α1/α2 antibodies (to detect Thr-172 phosphorylation and total AMPKα), or with anti-pACC and avidin (to detect Ser-79 phosphorylation and total ACC). The two probing reagents used in each of these dual-labelling protocols were labelled with different fluorescent dyes emitting in different regions of the infra-red (IR) spectrum, and the results were quantified in two separate channels using an IR laser scanner. In FIGS. 18B and 18C the results are expressed as ratios of the signal obtained using the phosphospecific antibody to the signal obtained for the total protein. This method corrects for different levels of expression of the proteins, and reveals that there was a good correlation between activation of AMPK and phosphorylation of Thr-172. There was also a correlation with phosphorylation of ACC, although in this case AICA riboside appeared to have a larger effect than phenformin.

Discussion

Our results provide strong evidence that LKB1-STRAD-MO25 complexes represent the major upstream kinase activities acting on AMPK. The key evidence may be summarised as follows:
1. During previous extensive efforts over 10 years to purify from rat liver extracts activities that activate dephosphorylated AMPK, ([14] and subsequent unpublished work), we have not detected any activities other than AMPKK1 and AMPKK2, at least under the assay conditions used.
2. Both AMPKK1 and AMPKK2 contained LKB1, STRADα and MO25α that were detectable by Western blotting and that correlated with the activity (FIG. 13).
3. Crucially, the ability of the AMPKK1 and AMPKK2 fractions to activate the AMPK α1 catalytic domain was almost completely eliminated by immunoprecipitation with anti-LKB1 antibody but not a control immunoglobulin. Activity was detected in the anti-LKB1 immunoprecipitates, together with the LKB1, STRADα and MO25α polypeptides, but not in the control immunoprecipitates (FIG. 14).
4. The AMPKK activity in AMPKK1 and AMPKK2 was not a contaminant that co-precipitated with anti-LKB1 antibody, because recombinant complexes of GST-LKB1, STRAD and MO25 expressed in 293 cells and purified on glutathione-Sepharose activated the AMPK α1 catalytic domain efficiently, and phosphorylated Thr-172 (FIG. 15). This activity was dependent on the integrity of the LKB1 catalytic domain, because complexes formed from catalytically inactive LKB1 with a STRAD and MO25 subunit failed to activate or phosphorylate AMPK. Phosphorylation of the α1 catalytic domain appeared to occur exclusively at Thr-172, because the wild type AMPKα1 catalytic domain, but not a T172A mutant, could be phosphorylated using [γ-³²P]ATP and the GST-LKB1-STRADα-MO25α complex.
5. Although most of the experiments in this paper were conducted using the bacterially expressed AMPK α1 catalytic domain as substrate, AMPKK1, AMPKK2 and recombinant LKB1-STRADα-MO25α complexes also activated heterotrimeric AMPK complexes (α1β1γ1 and α2β1γ1) efficiently, and phosphorylated them at Thr-172 on the α subunits (FIG. 16).
6. HeLa cells do not express LKB1 (FIG. 17) and therefore represent a natural “knock-out” cell line. The drugs AICA riboside and phenformin, which activate AMPK in other cell types via distinct mechanisms [33, 34], did not activate AMPK in HeLa cells. However, in cells stably transfected with DNA that expressed wild type LKB1 (but not a catalytically-inactive mutant) from an inducible promoter, the ability of AICA riboside and phenformin to activate AMPK, to phosphorylate Thr-172 on the AMPKα subunit, and to cause phosphorylation of a downstream target (ACC) was restored (FIG. 18). Although phenformin caused a larger activation of AMPK and phosphorylation of Thr-172 than AICA riboside in these cells, the reverse was true for phosphorylation of ACC. The reason for this is not known, but one possibility is that the two agents are differentially activating distinct pools of AMPK at different subcellular locations.

Although the LKB1 polypeptide was over-expressed in the HeLa cells, the degree of AMPK activation and the amount of STRADα and MO25α co-precipitating with LKB1 was actually lower than in 293 cells (FIG. 17). This strongly supports the findings in FIG. 15 showing that both accessory subunits are necessary to enhance the ability of LKB1 to activate AMPK, and also show that the active form of LKB1 is not being over-expressed compared to 293 cells. The results in the HeLa cells suggest that LKB1 and its accessory subunits are both necessary and sufficient for activation of AMPK in intact cells. However, they do not formally prove this, because if there are other kinases acting upstream of AMPK, it is conceivable that these are also deleted in HeLa cells. Our numerous attempts at purification of AMPKKs from rat liver extracts have only ever detected AMPKK1 and AMPKK2, which now appear to both represent LKB1-STRADα-MO25α complexes. However, as in any protein purification protocol the recovery of activity is not quantitative, and we cannot rule out the possibility that other upstream kinases could have been missed. We would also not detect any upstream kinase that was not active under the assay conditions used. For example, the assays were conducted in the presence of EGTA, so we would not detect any Ca²⁺-dependent protein kinases. We have already shown that a Ca²⁺/calmodulin-dependent protein kinase kinase (CaMKK) from pig brain can activate AMPK in cell-free assays [17], although it was much more effective at activating calmodulin-dependent protein kinase I, and there is no evidence that it phosphorylates AMPK in vivo.

Both AMPKK1 and AMPKK2 appear to contain LKB1, STRADα and MO25α, and thus it is not clear at present why they resolve on Q-Sepharose chromatography. One interesting difference was that the LKB1 polypeptide in AMPKK1 migrated significantly faster on SDS-PAGE than that in AMPKK2 (FIGS. 13 and 14). LKB1 is know to be phosphorylated at up to eight sites, and it is also farnesylated at Cys-433 near the C-terminus (34-37]. We therefore suspect that the difference in mobility may be due to a difference in covalent modification. Another difference between AMPKK1 and AMPKK2 was their Stokes radii estimated by size exclusion chromatography (5.7 versus 5.2 nm respectively). We previously estimated the Stokes radius for AMPKK1 to be 5.6 nm, and combining this with a sedimentation coefficient of 8.5S obtained by glycerol gradient centrifugation had obtained an estimate of 195 kDa for the molecular mass [14]. Assuming that AMPKK1 and AMPKK2 have a similar shape, this would yield a molecular mass of about 175 kDa for AMPKK2, which is reasonably close to the calculated molecular mass (not counting covalent modifications) of a 1:1:1 LKB1-STRADα-MO25α complex of 140 kDa. Although we cannot rule out the possibility that AMPKK1 contains an additional associated protein, it is possible that differences in covalent modification between AMPKK1 and AMPKK2 might affect the shape of the complex and hence the Stokes radius. Consistent with the behaviour of the AMPKK1 and AMPKK2 complexes on size exclusion chromatography, endogenous LKB1 was previously shown to migrate on gel filtration with an apparent molecular mass of 150-250 kDa, in marked contrast to LKB1 expressed in E. coli, which had an apparent molecular mass of 50 kDa [38]. Our present results confirm, using a probable physiological substrate, previous findings using an artificial substrate (myelin basic protein) that a STRAD subunit stimulates the kinase activity of LKB1 [30], and that the MO25 subunit stimulates the activity further, probably by stabilizing the LKB1-STRAD complex (Example 1). No AMPKK activity was obtained with recombinant LKB1 unless a STRAD subunit was also expressed, and the activity was increased substantially by the additional presence of an MO25 subunit (FIG. 15). It was also noticeable that the amount of STRADα and STRADβ that co-precipitated with LKB1 was greatly enhanced by the co-expression of MO25α or MO25β (FIG. 15) consistent with our previous findings (Example 1). The exact mechanism by which the STRAD and MO25 subunits activate LKB1 remains unclear, but these accessory subunits introduce scope for additional regulation of the kinase. It is already known that LKB1 phosphorylates STRADα at 2 distinct sites [30], and that STRADα and MO25α form a complex that retains LKB1 in the cytoplasm (Example 1). It is also interesting to note that both AMPK and its upstream kinase are heterotrimers.

Does activation of AMPK explain the ability of LKB1 to act as a tumour suppressor and to arrest cell growth and proliferation? This certainly seems plausible, because apart from the fact that AMPK is a general inhibitor of biosynthesis [1,2], there is also accumulating evidence that it can regulate progress through the cell cycle. For example, activation of AMPK inhibits proliferation of HepG2 cells by stabilizing p53 [13], and reduces the level of the RNA binding protein HuR in the cytoplasm, decreasing the stability of mRNAs encoding cyclins A and B1 [12]. Interestingly, expression of LKB1 in G361 cells that do not express the kinase causes an arrest in G1 phase that is associated with an induction of p21 and is dependent on p53 [23, 24].

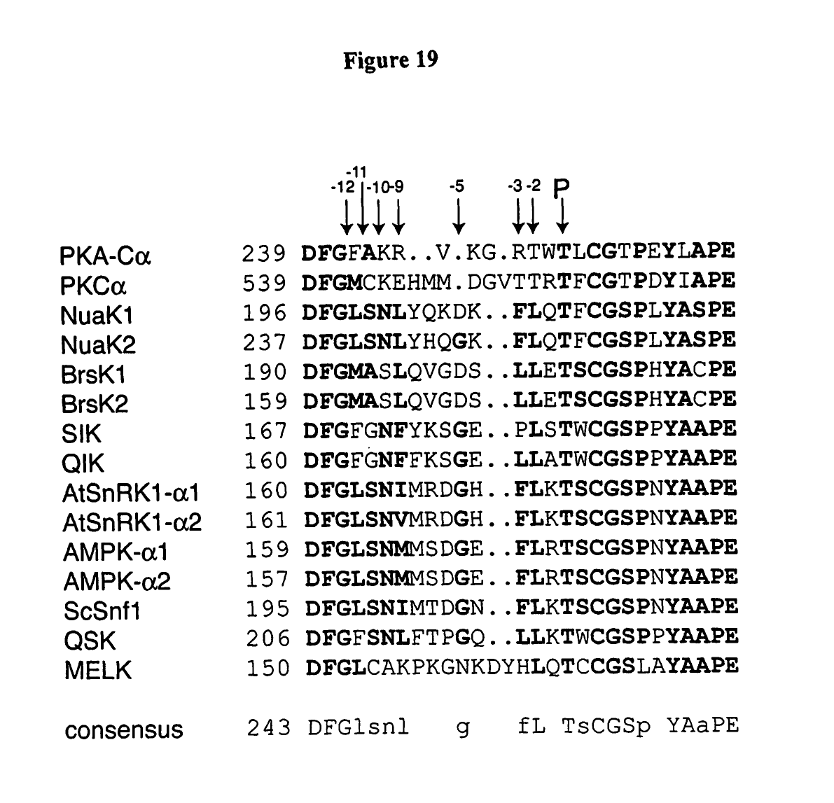

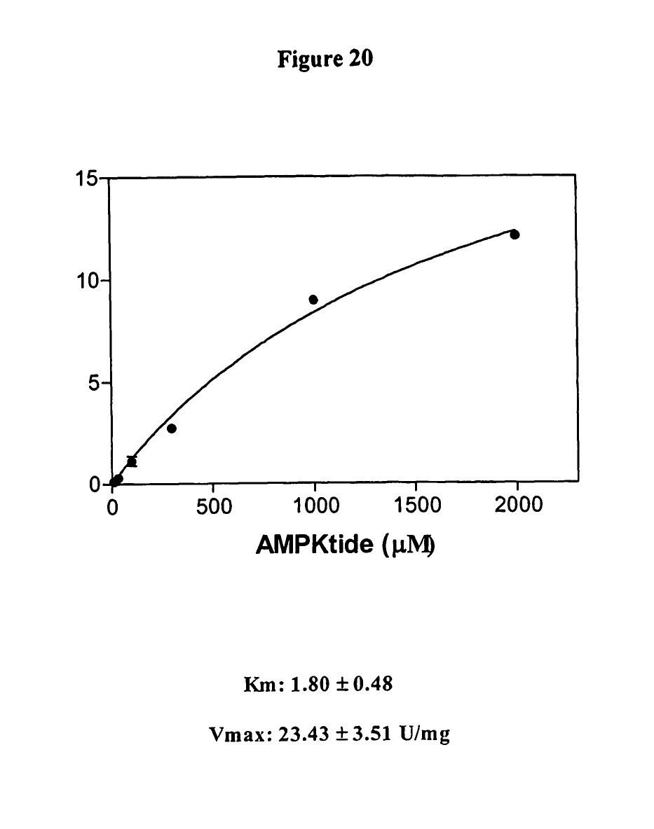

Another exciting possibility is that LKB1-STRAD-MO25 complexes might also act as upstream kinases for other protein kinases, in the same manner that PDK1 phosphorylates threonine residues in the activation loop of a number of kinases of the “AGC” sub-family [39]. A dendrogram showing the relationships between catalytic domain sequences of 518 human protein kinases encoded in the human genome [40] shows that the AMPKα1 and AMPKα2 subunits lie on a small sub-branch also containing eight other protein kinases (NuaK1, NuaK2, BrsK1, BrsK2, SIK, QIK, QSK and MELK; see [41] for details), most of which have either not been studied previously or about which very little is known. An alignment of the activation loop sequences of these kinases is shown in FIG. 19, together with those of the close relative “AGC” sub-family catalytic Ca subunit of cyclic AMP-dependent protein kinase (PKAα, which can be phosphorylated in the activation loop by autophosphorylation) and the a isoform of protein kinase C (PKCα), which is activated by phosphorylation in the activation loop by PDK1. Also included in FIG. 19 are the activation loop sequences of the Arabidopsis thaliana (AtSnRK1α1, AtSnRKα2) and Saccharomyces cerevisiae (ScSnf1) homologues of AMPK, both of which have been shown previously to be activated by rat liver AMPKK in cell-free assays [42, 43]. All of the sequences are well conserved on the C-terminal side of the threonine residue that is the target for phosphorylation, but the AMPK/Snf1 subfamily are more highly conserved on the N-terminal side between the invariant “DFG” motif and the threonine residue. Sequence features that are conserved in the AMPK/SNF1 subfamily but not in the “AGC” family include pairs of hydrophobic residues at the −3, −2 and the −9, −8 positions. NuaK1 and NuaK2 and the human, plant and yeast AMPK/SNF1 kinases also have a conserved “LSN” motif at the −12 to −10 positions. It remains to be determined whether the other human kinases in the AMPK sub-family are activated by phosphorylation of the equivalent activation loop threonine residue, and whether they might also be downstream targets for LKB1/STRAD/MO25 complexes. If this is the case, these other kinases could mediate some of the tumour suppressor functions of LKB1. Since the catalytic domains in this sub-family are closely related, but the other regions tend to be more variable, one could speculate that these kinases may be activated by different stresses or other stimuli via phosphorylation by LKB1/STRAD/MO25 complexes, and together may mediate the diverse cellular effects that are triggered by LKB1. It is also possible that they might share some common downstream targets with AMPK.

Conclusions

Our results provide strong evidence, both in cell-free assays and in intact cells, that complexes between LKB1, STRADα/β and MO25α/β constitute the long sought after upstream kinases that activate AMPK via phosphorylation at Thr-172 in the activation loop. Because it is already known that pharmacological activation of AMPK causes a general inhibition of biosynthesis, as well as a p53-dependent arrest in G1 phase, activation of AMPK by LKB1 might explain, at least in part, the ability of the latter to act as a tumour suppressor. LKB1 may also act as an upstream kinase for other members of the AMPK/SNF1 sub-family of protein kinases.

Material and Methods

Materials, Proteins and Antibodies

Protein G-Sepharose and prepacked Q-Sepharose columns were from Amersham Pharmacia Biotech, UK. GST-AMPKα1-catalytic domain was expressed in E. coli and purified as described previously [44]. Sheep antibodies against the α1 and α2 subunits of AMPK [45], human LKB1, MO25α and MO25β (Example 1), and phosphospecific antibody against the Thr-172 site on the AMPK α subunit (anti-pT172) [46] were described previously. Sheep antibody against AMPKα1-catalytic domain was raised against the peptide CDPMKRATpIKDIRE (cysteine+residues 252-264 of rat α1, Tp=phosphothreonine) using methods described for anti-pT172 [46]. Although designed as a phosphospecific antibody, it recognizes GST-AMPKα1-catalytic domain expressed in bacteria and recognition is not affected by protein phosphatase treatment. The monoclonal antibody against STRADα was described previously [30]. Sources of other materials and proteins were describe previously [14].

Enzyme Assays

AMPK [14]and AMPKK [34] were assayed, and units defined, as described previously. Rapid lysis of cells for AMPK assays was as described previously [47]. Assays were carried out in triplicate and results are expressed as mean ± standard deviation.

Purification of AMPKK1 and AMPKK2

AMPKK was purified to the Blue-Sepharose stage as described previously [14]. The flow-through from this column was adjusted to 160 mM NaCl by dilution in buffer A (50 mM Hepes, 10% (w/v) glycerol, 0.02% (w/v) Brij-35, 1 mM EDTA, 1 mM EGTA, 1 mM DTT, 1 mM benzamidine, 0.1 mM PMSF, 1 μg/ml soybean trypsin inhibitor), and applied to a high performance Q-Sepharose HiLoad 16/10 column in buffer A plus 160 mM NaCl at 3 ml/min. The column was washed in buffer A plus 160 mM NaCl until the A₂₈₀ was <0.05, and AMPKK activity was then eluted with a linear gradient (120 ml) from 160-400 mM NaCl in buffer A.

Expression of Recombinant LKB1 Complexes in HEK-293T Cells

Various combinations of GST-tagged LKB1, FLAG-tagged STRADα or STRADβ, and myc-tagged MO25α or MO25β were expressed in HEK-293T cells and the complexes purified on glutathione-Sepharose as described in Example 1.

Phosphorylation of GST-α1 Catalytic Domain Using [γ-³²P]ATP

GST-α1 catalytic domain (50 μg/ml), either wild type or a T172A mutant [44] was incubated for 30 min at 30° C. with 5 mM MgCl₂ and [γ-³²P]ATP (200 μM; ˜750 cpm/pmole) in the presence or absence of the GST-LKB1:STRADα:MO25α complex (20 units/ml). The reaction was terminated by the addition of SDS sample buffer (Invitrogen), the polypeptides resolved by SDS-PAGE and the dried gel subjected to autoradiography.

Preparation of Recombinant AMPK Hterotrimers

Plasmids encoding myc-α1, β1 and γ1 were co-expressed in CCL13 cells (45], and cells harvested by the rapid lysis method [47]. Lysates were immunoprecipitated with anti-myc antibody and resuspended in 50 mM NaHEPES, 1 mM dithiothreitol, 0.02% (w/v) Brij-35, pH 7.5.

Immunoprecipitation of Endogenous LKB1

Immunoprecipitation of endogenous LKB1 using anti-human antibody and protein G-Sepharose is described in Example 1. The kinase kinase assays were conducted using GST-α1-catalytic domain as substrate in shaking incubators as described previously for immunoprecipitate assays of AMPK [47].

HeLa Cells Expressing LKB1

The generation of HeLa cells stably expressing inducible (Tet-ON) wild type or kinase-inactive mutant LKB1, and conditions for their culture, has been described previously [32].

Protein Analysis and Electrophoresis

Protein concentration was determined using the dye-binding method of Bradford [48]. SDS-PAGE was achieved using precast Bis-Tris 412% gradient polyacrylamide gels, in the MOPS buffer system (Invitrogen), except for analysis of acetyl-CoA carboxylase, where pre-cast 3-8% Tris-acetate gels (Invitrogen) were used. Proteins were transferred to nitrocellulose membranes (BioRad) using the Xcell II Blot Module (Invitrogen).

Detection of Western Blots by Infra-Red Imaging

To analyse phosphorylation of ACC, samples were analysed by SDS-PAGE, transferred to nitrocellulose (BioRad) and incubated in Odyssey™ Blocking buffer containing 0.2% Tween-20 for 1 h. Anti-pACC antibody (1.46 μg/ml in blocking buffer) was then added and left shaking for 1 h. The membranes were washed 6×5 min with TBS (10 mM Tris-HCl, pH 7.4, 0.5M NaCl) plus Tween-20 (0.2%). The membranes were immersed in blocking buffer containing 0.2% Tween-20 and 1 μg/ml anti-sheep IgG conjugated to JR dye 680 (Molecular Probes, Leiden, The Netherlands) and 1 μg/ml streptavidin conjugated to IR Dye 800 (Rockland Inc., Philadelphia, Pa.) and left shaking for 1 h, protected from light. The membranes were then washed 6×5 min using TBS-Tween (0.2%) and 1×5 min in PBS. The membranes were scanned in two different channels using the Odyssey™ IR imager, the results quantified using Odyssey™ software and expressed as a ratio of the signal obtained with the pACC antibody to that obtained with streptavidin. Analysis of phosphorylation of GST-α1 catalytic domain was similar except that the 4-12% Bis-Tris gels were used, and the membranes were simultaneously probed for 1 h with the sheep anti-pT172 and anti-AMPKα1-catalytic domain antibodies, directly labelled with the IR dye 680 and IR Dye 800 respectively, according to manufacturers' instructions.

### REFERENCES

1. Hardie, D G, Scott, J W, Pan, D A, Hudson, E R: Management of cellular energy by the AMP-activated protein kinase system. FEBS Lett. 2003 546, 113-120.
2. Hardie, D G, Hawley, S A: AMP-activated protein kinase: the energy charge hypothesis revisited. BioEssays 2001, 23: 1112-1119.
3. Coven, D L, Hu, X, Cong, L, Bergeron, R, Shulman, G I, Hardie, D G, Young, L H: Physiologic role of AMP-activated protein kinase (AMPK) in the heart: graded activation during exercise. Am J Physiol Endocrinol Metab 2003,
4. Horman, S et al.: Activation of AMP-activated protein kinase leads to the phosphorylation of Elongation Factor 2 and an inhibition of protein synthesis. Current Biol. 2002, 12: 1419-1423.
5. Krause, U, Bertrand, L, Hue, L: Control of p70 ribosomal protein S6 kinase and acetyl-CoA carboxylase by AMP-activated protein kinase and protein phosphatases in isolated hepatocytes. Eur. J. Biochem. 2002, 269: 3751-3759.
6. Bolster, D R, Crozier, S J, Kimball, S R, Jefferson, L S: AMP-activated protein kinase suppresses protein synthesis in rat skeletal muscle through downregulated mTOR signaling. J. Biol. Chem. 2002, 277: 23977-23980.
7. Meisse, D, Van de Casteele, M, Beauloye, C, Hainault, I, Kefas, B, Rider, M, Foufelle, F, Hue, L: Sustained activation of AMP-activated protein kinase induces c-Jun N-terminal kinase activation and apoptosis in liver cells. FEBS Lett. 2002, 526: 38-42.
8. Kefas, B A, Cai, Y, Ling, Z, Heimberg, H, Hue, L, Pipeleers, D, Van De Casteele, M: AMP-activated protein kinase can induce apoptosis of insulin-producing MIN6 cells through stimulation of c-Jun-N-terminal kinase. J. Mol. Endocrinol. 2003, 30: 151-161.
9. Kefas, B A, Heimberg, H, Vaulont, S, Meisse, D, Hue, L, Pipeleers, D, Van De Casteele, M: AICA-riboside induces apoptosis of pancreatic beta cells through stimulation of AMP-activated protein kinase. Diabetologia 2003, 46: 250-254.
10. Stefanelli, C, Stanic, I, Bonavita, F, Flamigni, F, Pignatti, C, Guarnieri, C, Caldarera, C M: Inhibition of glucocorticoid-induced apoptosis with 5-aminoimidazole-4-carboxamide ribonucleoside, a cell-permeable activator of AMP-activated protein kinase. Biochem. Biophys. Res. Commun. 1998, 243: 821-826.
11. Blazquez, C, Geelen, M J, Velasco, G, Guzman, M: The AMP-activated protein kinase prevents ceramide synthesis de novo and apoptosis In astrocytes. FEBS Lett. 2001, 489: 149-153.
12. Wang, W et al.: AMP-activated kinase regulates HuR subcellular localization. Mol. Cell. Biol. 2002, 27: 3425-3436.
13. Imamura, K, Ogura, T, Kishimoto, A, Kaminishi, M, Esumi, H: Cell Cycle Regulation via p53 Phosphorylation by a 5′-AMP Activated Protein Kinase Activator, 5-Aminoimidazole-4-Carboxamide-1-beta-d-Ribofuranoside, in a Human Hepatocellular Carcinoma Cell Line. Biochem. Biophys. Res. Commun. 2001, 287: 562-567.
14. Hawley, S A, Davison, M, Woods, A, Davies, S P, Beri, R K, Carling, D, Hardie, D G: Characterization of the AMP-activated protein kinase kinase from rat liver, and identification of threonine-172 as the major site at which it phosphorylates and activates AMP-activated protein kinase. J. Biol. Chem. 1996, 271: 27879-27887.
15. Sutherland, C M, Hawley, S A, Stark, M J R, Hardie, D G: Identification of Elm1p as one of three upstream kinases for the Saccharomyces cerevisiae SNF1 complex. Curr. Biol. 2003: submitted.
16. Nath, N, McCartney, R R, Schmidt, M C: Yeast Pak1 kinase associates with and activates Snf1 . Mol. Cell. Biol. 2003, 23: 3909-3917.
17. Hawley, S A, Selbert, M A, Goldstein, E G, Edelman, A M, Carling, D, Hardie, D G: 5′-AMP activates the AMP-activated protein kinase cascade, and Ca²⁺/calmodulin the calmodulin-dependent protein kinase I cascade, via three independent mechanisms. J. Biol. Chem. 1995, 270: 27186-27191.
18. Hemminki, A et al.: A serine/threonine kinase gene defective in Peutz-Jeghers syndrome. Nature 1998, 391: 184-187.
19. Jenne, D E, Reimann, H, Nezu, J, Friedel, W, Loff, S, Jeschke, R, Muller, O, Back, W, Zimmer, M: Peutz-Jeghers syndrome is caused by mutations in a novel serine threonine kinase. Nat. Genet. 1998, 18: 38-43.
20. Hemminki, A: The molecular basis and clinical aspects of Peutz-Jeghers syndrome. Cell. Mol. Life Sci. 1999, 55: 735-750.
21. Giardiello, F M, Brensinger, J D, Tersmette, A C, Goodman, S N, Petersen, G M, Booker, S V, Cruz-Correa, M, Offerhaus, J A: Very high risk of cancer in familial Peutz-Jeghers syndrome. Gastroenterology 2000, 119: 1447-1453.
22. Boudeau, J, Sapkota, O, Alessi, D R: LKB1, a protein kinase regulating cell proliferation and polarity. FEBS Lett. 2003, 546: 159-165.
23. Tiainen, M, Ylikorkala, A, Makela, T P: Growth suppression by LKB1 is mediated by a G(1) cell cycle arrest. Proc. Natl. Acad. Sci. USA 1999,96:9248-9251.
24. Tiainen, M, Vaahtomeri, K, Ylikorkala, A, Makela, T P: Growth arrest by the LKB1 tumor suppressor: induction of p21(WAF1/CIP1). Hum. Mol. Genet. 2002, 11:1497-1504.
25. Jishage, K et al.: Role of Lkb1, the causative gene of Peutz-Jegher's syndrome, in embryogenesis and polyposis. Proc. Natl. Acad. Sci. USA 2002, 99: 8903-8908.
26. Bardeesy, N, Sinha, M, Hezel, A F, Signoretti, S, Hathaway, N A, Sharpless, N E, Loda, M, Carrasco, D R, DePinho, R A: Loss of the LKB1 tumour suppressor provokes intestinal polyposis but resistance to transformation. Nature 2002, 419: 162-167.
27. Miyoshi, H, Nakau, M, Ishikawa, T O, Seldin, M F, Oshima, M, Taketo, M M: Gastrointestinal hamartomatous polyposis In LKB1 heterozygous knockout mice. Cancer Res. 2002, 62: 2261-2266.
28. Rossi, D J et al.: Induction of cyclooxygenase-2 in a mouse model of Peutz-Jeghers polyposis. Proc. Natl. Acad. Sci. USA 2002, 99: 12327-12332.
29. Nakau, M, Miyoshi, H, Seldin, M F, Imamura, M, Oshima, M, Taketo, M M: Hepatocellular carcinoma caused by loss of heterozygosity in LKB1 gene knockout mice. Cancer Res. 2002, 62: 4549-4553.
30. Baas, A F, Boudeau, J, Sapkota, G P, Smit, L, Medema, R, Morrice, N A, Alessi, D R, Clevers, H C: Activation of the tumour suppressor kinase LKB1 by the STE20-like pseudokinase STRAD. EMBO J. 2003, 22: 3062-3072.
31. Boudeau, J, Baas, A F, FDeak, M, Morrice, N A, Kieloch, A, Schutowski, M, Prescott, A R, Clevers, H C, Alessi, D R: MO25 isoforms interact with the STE20-related psedokinase STRADα/p and enhance their ability to bind, activate and localise the LKB1 tumour suppressor in the cytoplasm. EMBO J. 2003: submitted; Example 1.
32. Corton, J M, Gillespie, J G, Hawley, S A, Hardie, D G: 5-Aminoimidazole-4-carboxamide ribonucleoside: a specific method for activating AMP-activated protein kinase in intact cells? Eur. J. Biochem. 1995, 229: 558-565.
33. Hawley, S A, Gadalla, A E, Olsen, G S, Hardie, D G: The anti-diabetic drug metformin activates the AMP-activated protein kinase cascade via an adenine nucleotide-independent mechanism. Diabetes 2002, 51: 2420-2425.
34. Sapkota, G P, Deak, M, Kieloch, A, Morrice, N, Goodarzi, A A, Smythe, C, Shiloh, Y, Lees-Miller, S P, Alessi, D R: Ionizing radiation induces ataxia telangiectasia mutated kinase (ATM)-mediated phosphorylation of LKB1/STK11 at Thr-366. Biochem. J. 2002, 368: 507-516.
35. Collins, S P, Reoma, J L, Gamm, D M, Uhler, M D: LKB1, a novel serine/threonine protein kinase and potential tumour suppressor, is phosphorylated by cAMP-dependent protein kinase (PKA) and prenylated in vivo. Biochem. J. 2000, 345 Pt 3: 673-680.
36. Sapkota, G P, Kieloch, A, Lizcano, J M, Lain, S, Arthur, J S, Williams, M R, Morrice, N, Deak, M, Alessi, D R: Phosphorylation of the protein kinase mutated In Peutz-Jeghers cancer syndrome, LKB1/STK11, at Ser431 by p90(RSK) and cAMP-dependent protein kinase, but not its farnesylation at Cys(433), is essential for LKB1 to suppress cell growth. J. Biol. Chem. 2001, 276: 19469-19482.
37. Sapkota, G P, Boudeau, J, Deak, M, Kieloch, A, Morrice, N, Alessi, D R: Identification and characterization of four novel phosphorylation sites (Ser31, Ser325, Thr336 and Thr366) on LKB1/STK11, the protein kinase mutated in Peutz-Jeghers cancer syndrome. Biochem. J. 2002, 362: 481-490.
38. Boudeau, J, Deak, M, Lawlor, M A, Morrice, N A, Alessi, D R: Heat-shock protein 90 and Cdc37 interact with LKB1 and regulate its stability. Biochem. J. 2003, 370: 849-857.
39. Alessi, D R: Discovery of PDK1, one of the missing links in insulin signal transduction. Colworth Medal Lecture. Biochem. Soc. Trans. 2001, 29: 1-14.
40. Manning, G, Whyte, D B, Martinez, R, Hunter, T, Sudarsanam, S: The protein kinase complement of the human genome. Science 2002, 298: 1912-1934.
41. Kinase.com [http://www.kinase.com/]
42. Sugden, C, Donaghy, P G, Halford, N G, Hardie, D G: Two SNF1-related protein kinases from spinach leaf phosphorylate and inactivate HMG-CoA reductase, nitrate reductase and sucrose phosphate synthase in vitro. Plant Physiol. 1999, 120: 257-274.
43. Wilson, W A, Hawley, S A, Hardie, D G: The mechanism of glucose repression/derepression in yeast: SNF1 protein kinase is activated by phosphorylation under derepressing conditions, and this correlates with a high AMP:ATP ratio. Current Biol. 1996, 6: 1426-1434.
44. Scott, J W, Norman, D G, Hawley, S A, Kontogiannis, L, Hardie, D G: Protein kinase substrate recognition studied using the recombinant catalytic domain of AMP-activated protein kinase and a model substrate. J. Mol. Biol. 2002, 317: 309-323.
45. Woods, A, Salt, 1, Scott, J, Hardie, D G, Carling, D: The a1 and a2 isoforms of the AMP-activated protein kinase have similar activities in rat liver but exhibit differences in substrate specificity in vitro. FEBS Lett. 1996, 397: 347-351.
46. Sugden, C, Crawford, R M, Halford, N G, Hardie, D G: Regulation of spinach SNF1-related (SnRK1) kinases by protein kinases and phosphatases is associated with phosphorylation of the T loop and is regulated by 5′-AMP. Plant J. 1999, 19: 1-7.
47. Hardie, D G, Salt, I P, Davies, S P: Analysis of the role of the AMP-activated protein kinase in the response to cellular stress. Methods Mol. Biol. 2000, 99: 63-75.
48. Bradford, M M: A rapid and sensitive method for the quantitation of microgram quantities of protein utilizing the principle of protein-dye binding. Anal. Biochem. 1976, 72: 248-254.
49. Devereux, J, Haeberli, P, Smithies, O: A comprehensive set of sequence analysis programs for the VAX. Nuc. Acids Res. 1984, 12: 387-395.
50. BOXSHADE v3.21 [http://www.ch.embnet.org/software/BOX_form.html]

### EXAMPLE 3

### Peptide Substrate for LKB1

A kinase assay for LKB1 can be performed using a peptide substrate, for example using the T-loop peptide of AMPK whose sequence is

LSNMMSDGEFLRTSCGSPNRRR

(HUMAN AMPKα1 pha-1 Residues 163-181 with 3 additional Arg residues added to the C-terminal to enable binding to P81 paper).

An assay may be performed as follows. Other assays using such a substrate peptide may be used, for example in a high throughput format. The standard assay (50 μl) contained: 0.3 μg of purified GST-LKB1-STRADα-MO25α complex, 50 mM Tris/HCl pH 7.5, 0.1 mM EGTA, 0.1% (by vol) 2-mercaptoethanol, 10 mM magnesium acetate, 0.1 mM [γ³²P]ATP (˜200 cpm/pmol) and T-loop peptide (LSNMMSDGEFLRTSCGSPRRR, 10-2000 μM). The assays were carried out for 30 min at 30° C., then terminated by pipetting the reaction mixture onto a 1 cm square of p81 paper, which was washed with 6 changes of 250 ml of 50 mM phosphoric acid (2 min each wash), the paper washed once in 200 ml of acetone and dried. Radioactivity bound to the p81 paper was quantitated by Cherenkov counting. 1 unit of activity is the amount of enzyme that catalyses the phosphorylation of 1 nmol of peptide per minute.

The Kₘ for the peptide was estimated to be 1.80±0.48 and the Vₘₐₓ 23.4±3.5

### EXAMPLE 4

### Activation of AMPK-Related Kinases by LKB1

12 other protein kinases (NUAK1, NUAK2, BRSK1, BRSK2, QIK, QSK, SIK, MARK1, MARK2, MARK3, MARK4 and MELK) are closely related to the AMP-activated protein kinase (AMPK). Here we demonstrate that LKB1 can phosphorylate the T-loop of all members of the AMPK-subfamily, (not demonstrated for MELK), increasing their activity >50-fold. LKB1 catalytic activity and the presence of MO25 and STRAD are required to activate all the AMPK-related kinases. Mutation of the T-loop. Thr phosphorylated by LKB1 to Ala prevented activation, while mutation to Glu produced constitutively active forms of many of the AMPK-related kinases. We developed assays to measure the activities of endogenous NUAK2, QIK, QSK, SIK, MARK1, MARK2/3 and MARK4 kinases in cells expressing LKB1, and exploited these to show that the activities of these enzymes were markedly reduced in LKB1^{-/-} fibroblasts or in HeLa cells that do not express LKB1. Interestingly, neither LKB1 activity, nor that of any of the AMPK-related kinases is stimulated by phenformin or AICA riboside, two drugs that induce the phosphorylation and activation of AMPK. Our results show that LKB1 functions as a master upstream protein kinase, regulating the activities of the AMPK-related kinases as well as AMPK. Between them, these kinases may mediate some of the physiological effects of LKB1, including its tumour suppressor function.

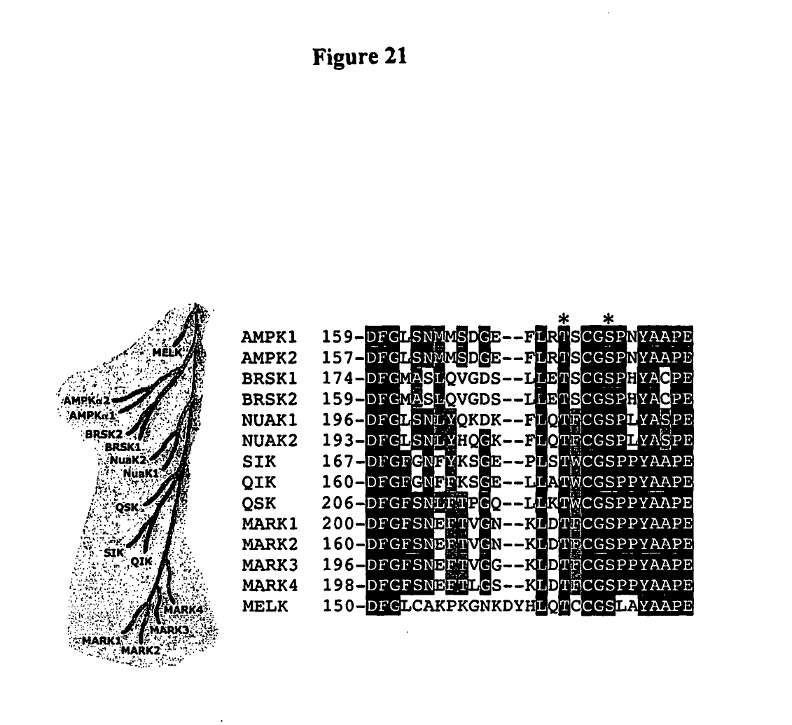

Inspection of the human kinome indicates that there are 12 protein kinases (BRSK1, BRSK2, NUAK1, NUAK2, QIK, QSK, SIK, MARK1, MARK2, MARK3, MARK4 and MELK) that are closely related to AMPKα1 and AMPKα2 (FIGS. 21A and B). The nomenclature we will employ for the “AMPK-related kinases” is based upon that used by Manning and colleagues (Manning et al., 2002). MARK3 is also known as PAR1A or C-TAK1 (Peng et al., 1998; Spicer et al., 2003), NUAK1 as ARK5 (Suzuki et al., 2003b), NUAK2 as SNARK (Lefebvre et al., 2001) and QIK as SIK2 (Horike et al., 2003). The T-loop Thr residue of AMPKα1 and AMPKα2 that is phosphorylated by LKB1, and many of the surrounding residues, are conserved in the AMPK-related kinases (FIGS. 21A & B). This indicates that LKB1 might also phosphorylate the T-loop Thr residue and hence regulate the AMPK-related kinases in the same way that PDK1 regulates the activity of a group of related AGC kinases (Alessi, 2001). The MARK kinases have been proposed to play key roles in controlling cell polarity and are known to be regulated by phosphorylation of their T-loop Thr residue (Lrewes and Nurse, 2003; Spicer et al., 2003). Studies in Drosophila initially indicated that MARK3 might lie upstream of LKB1 (Martin and St Johnston, 2003), but recent work in mammalian cells disputed this conclusion and suggested that LKB1 lies upstream of MARK3 and controls its activity (Spicer et al., 2003). Little is known about the function or mechanism of regulation of the remaining AMPK-related enzymes. In this Example we therefore sought to investigate the role that LKB1 plays in regulating the activity of the AMPK-related kinases.

Results

Activation of AMPK-related kinases by the LKB1 complex. We cloned and expressed in E. Coli the full-length versions of all of the twelve AMPK-related kinases and developed assays for these enzymes employing the AMARA AMPK peptide substrate for AMPK (Dale et al., 1995), which is not phosphorylated by the LKB1 complex (JML, data not shown). We found that all of the AMPK-related kinases purified from E. coli, phosphorylated this peptide and possessed low basal activities of <1 U/mg (1 Unit ═I nmol peptide phosphorylated per min), with the exception of MELK, which had an activity of ˜40 U/mg (FIG. 21). Following incubation of the AMPK-related kinases with the LKB1:STRAD:MO25 complex and MgATP, the activity of the AMPK-related kinases, was increased 50 to 200-fold, except for MELK whose activity was hardly increased at all (FIG. 21). Catalytically inactive LKB1 complexed to STRADα and MO25α failed to increase the basal activity of the AMPK-related kinases, indicating that the kinase activity of LKB1 was required (FIG. 21).

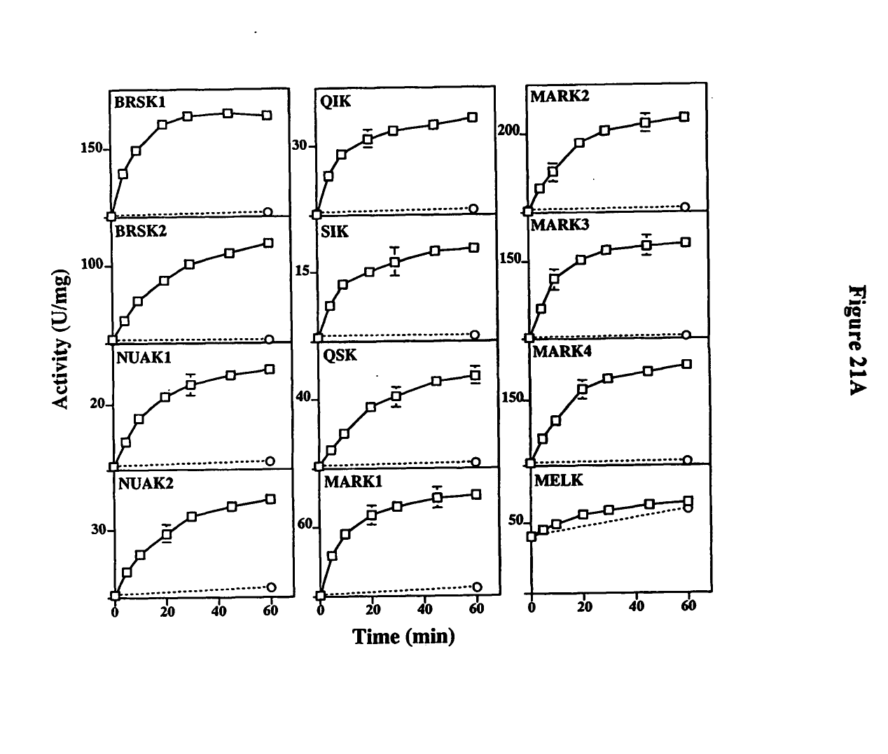

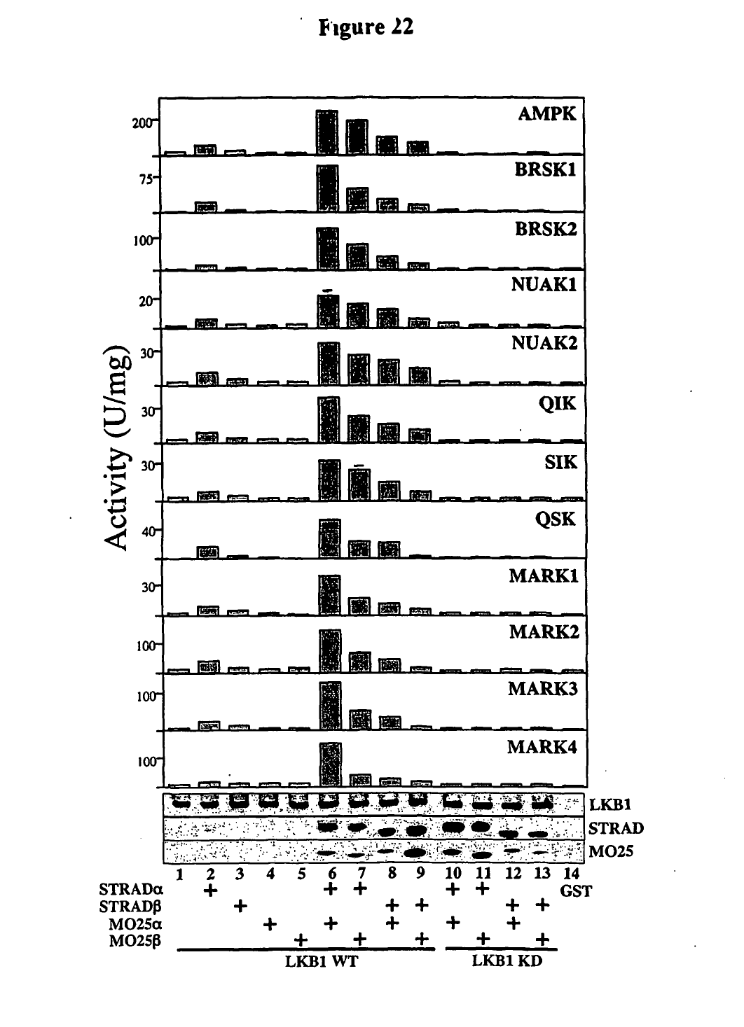

Activation of AMPK-related kinases by LKB1 requires STRAD and MO25. To determine the importance of the STRAD and MO25 subunits in enabling LKB1 to activate the AMPK-related kinases, we expressed GST-tagged LKB1, FLAG-tagged STRADα/β and myc-tagged MO25α/β in various combinations in 293 cells, and affinity purified the complexes on glutathione-Sepharose (FIG. 22). The purified complexes were incubated in the presence of MgATP with the AMPK-related kinases and their activation was measured. LKB1 on its own did not activate any of the AMPK-related kinases significantly (FIG. 22, compare lanes 1 and 14). The same result was obtained with LKB1 that had been co-expressed with MO25α or MO25β (FIG. 22, lanes 4 and 5). This was as expected, since these proteins do not interact with LKB1 in the absence of STRADα/β (Example 1; Boudeau et al., 2003a). An LKB1:STRADα complex gave a small activation (FIG. 22, compare lanes 1 and 2), but a heterotrimeric complex containing LKB1, STRADα or STRADβ, and MO25α or MO25β was required to obtain a large activation (FIG. 22, lanes 6 to 9). As previously reported for AMPKα1 (FIG. 22 and Example 2; Hawley et al., 2003), all AMPK-related kinases were activated most efficiently by the LKB1:STRADα:MO25α complex, although the relative effectiveness of different heterotrimeric complexes varied from substrate to substrate (FIG. 22). Heterotrimeric complexes containing a catalytically inactive mutant of LKB1 were unable to activate any enzyme (FIG. 22, lanes 10-13). It should be noted that when STRAD isoforms were co-expressed with LKB1 in the absence of MO25, the amount of STRAD co-precipitated with LKB1 was markedly reduced (FIG. 22 compare lanes 2 and 3 with lanes 6 and 7), as MO25 was required to stabilise the LKB1:STRAD complex (Example 1; Boudeau et al., 2003a).

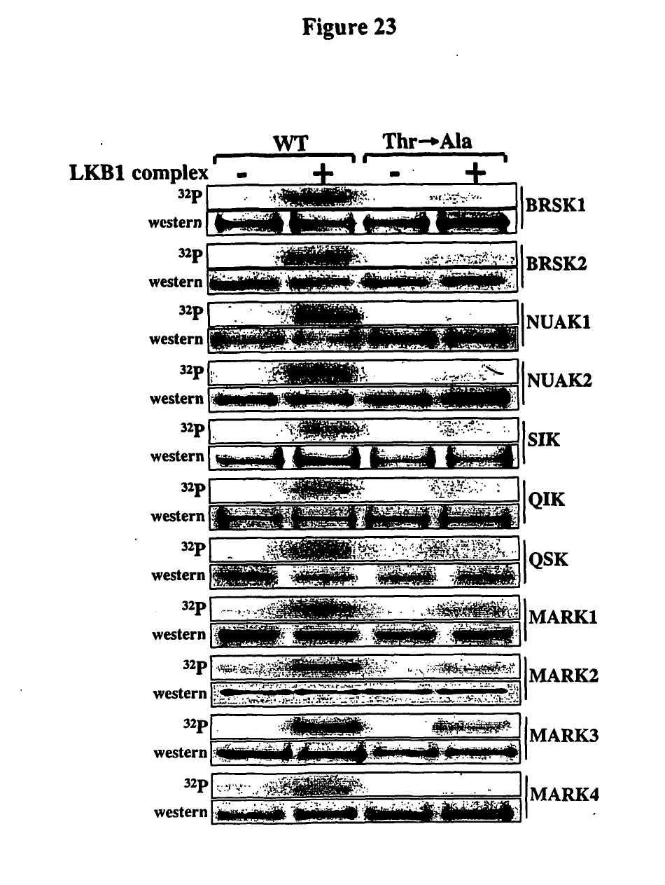

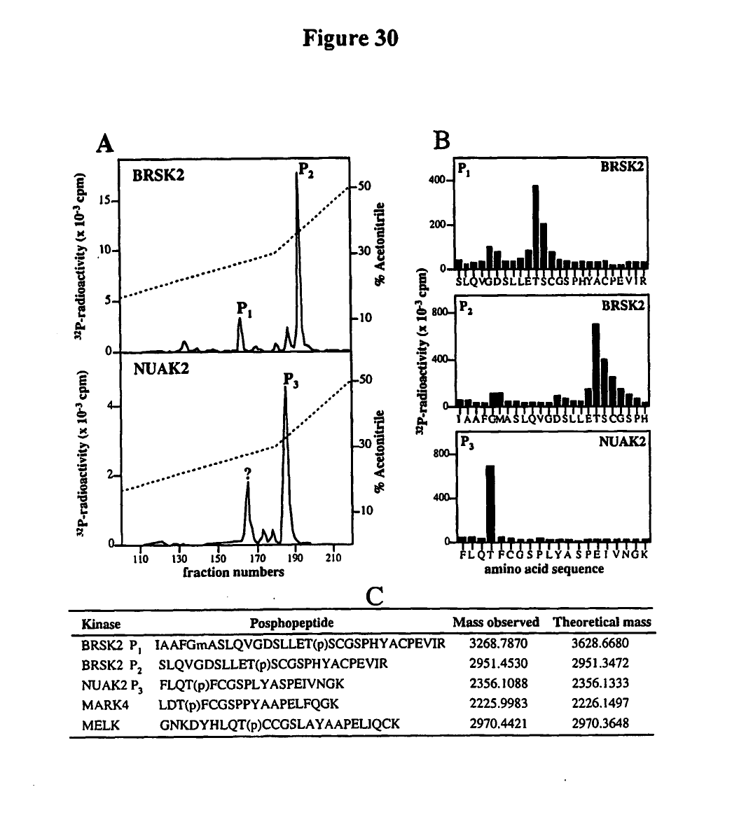

The LKB1 complex phosphorylates AMPK-related kinases at the activation loop. As the LKB1 complex was previously shown to phosphorylate AMPKα1 specifically at Thr172 in its activation loop, we first mutated the equivalent T-loop residue in the AMPK-related kinases (FIG. 21A & B) to Ala, and tested how this affected phosphorylation of these enzymes by the LKB1 complex. We demonstrated that the wild type AMPK-related kinases were phosphorylated by the LKB1 complex and mutation of the T-loop Thr residue to Ala abolished or significantly reduced phosphorylation (FIG. 23). We also performed detailed phosphorylation site analysis of catalytically inactive BRSK2 and NUAK2 mutants that had been phosphorylated in vitro by the LKB1 complex (FIG. 30). The ³²P-labelled BRSK2 and NUAK2 proteins were digested with trypsin and the resulting peptides separated by chromatography on a C₁₈ column. The major ³²P-labelled peptides were analysed by MALDI TOF-TOF mass spectrometry and solid phase Edman sequencing, which demonstrated that these enzymes were phosphorylated at their T-loop Thr residue (FIG. 30).

T-loop phosphorylation activates AMPK-related kinases. To study the role that T-loop phosphorylation of BRSK1, BRSK2, NUAK1, NUAK2, QIK, QSK, SIK and MELK plays in their regulation, the T-loop Thr residues of these enzymes were mutated to either Ala to prevent phosphorylation, or Glu to mimic phosphorylation. In all cases, the basal activity of the Ala mutants was low and their activity not increased by incubation with the LKB1 complex (FIG. 24). In contrast, the basal activity of the Glu mutants was similar to that of the wild type enzyme phosphorylated by LKB1, and was not further increased following incubation with the LKB1 complex and MgATP (FIG. 24). In the case of MELK, the Ala mutant possessed low activity and the Glu mutant was of similar activity to the wild type enzyme, suggesting that wild type MELK (like other AMPK-related kinases) required T-loop phosphorylation for activity. As wild type MELK expressed in E. coli was active MELK may be able to catalyse phosphorylation of its own T-loop Thr residue. Consistent with this, MALDI TOF-TOF analysis of wild type MELK expressed in E. coli established that the T-loop was indeed phosphorylated (FIG. 30C).

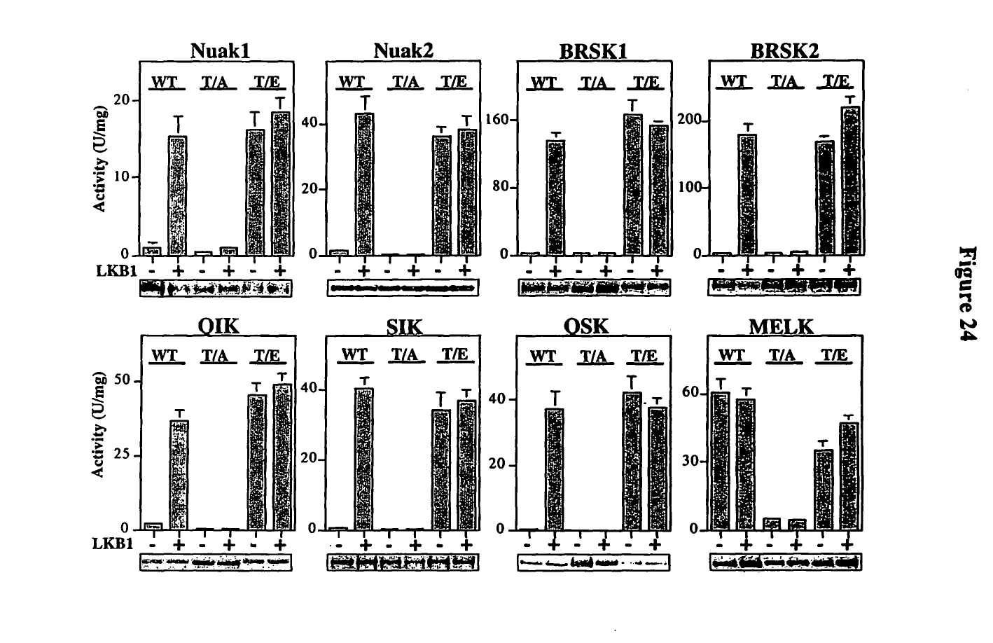

Phosphorylation and activation of the MARK kinases by LKB1. Previous studies have revealed that the MARK3 and MARK3 kinases, in addition to being phosphorylated at the T-loop Thr residue, are also phosphorylated at a nearby Ser residue (FIGS. 21A and B and (Drewes et al., 1997; Johnson et al., 1996; Timm et al., 2003)). Peptide-mapping of catalytically inactive MARK3 phosphorylated by the LKB1 complex, revealed that both the Thr211 and Ser215 residues within the T-loop were phosphorylated (FIG. 25A). Interestingly, catalytically inactive MARK3[T211A] mutant was not phosphorylated at Ser215 by LKB1, but the catalytically inactive MARK3(S215A] mutant was still phosphorylated at Thr211 (FIG. 25A). We next investigated how mutation of the T-loop Thr or Ser residue affected activation of MARK3 by the LKB1 complex (FIG. 25C). The MARK3[T211A] mutant could not be activated by the LKB1 complex but the MARK3[S215A] mutant was activated to a small extent. The MARK3[T211E] mutant possessed only ˜15% of the activity of the wild type enzyme activated by the LKB1 complex and could not be activated further by LKB1. The MARK3[S215E] mutant was inactive and could not be activated by LKB1 (FIG. 25C). As expected, we also found that mutation of the T-loop Thr to Ala in MARK1, MARK2 and MARK4 prevented their activation by LKB1 (FIG. 25D to F). Mutation of the T-loop Thr residue to Glu in MARK1, MARK2 and MARK3, resulted in either no activation or only a small activation of these enzymes (FIG. 25D to F). In contrast, the equivalent mutation in MARK4, vastly increased activity (FIG. 25F). Peptide-mapping studies and MALDI TOF-TOF mass spectrometry demonstrated that LKB1 phosphorylated the MARK4 T-loop at only the Thr residue and not the Ser residue (FIG. 25G and FIG. 30C).

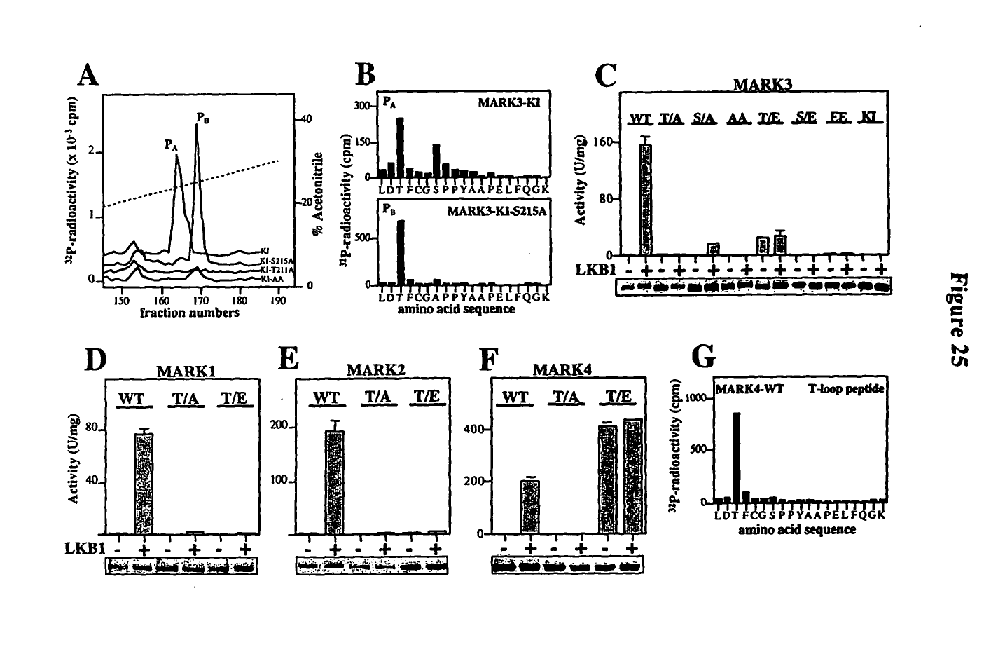

Identification of peptide substrates for LKB1. We next tested whether the LKB1 complex was able to phosphorylate peptides encompassing the activation loop of AMPKα1, BRSK2, NUAK2, SIK, MARK3 and MELK. All the peptides were phosphorylated, but with differing efficiencies (FIG. 26A). Solid phase Edman sequencing confirmed that every peptide was specifically phosphorylated by LKB1 at the T-loop Thr residue (FIG. 26B). The optimal peptide substrate derived from the T-loop of NUAK2 was phosphorylated by LKB1 with a Kₘ value of 150 μM and was a better substrate than the peptide derived from the T-loop of AMPKα1 (Kₘ, 1.4 mM) (FIG. 26A). The MELK peptide was the poorest substrate. We also assayed the different combinations of LKB1 STRADα/β and MO25α/β complexes used in FIG. 22, with the T-loop peptides as substrates, and the pattern of LKB1 activity mirrored that observed using the full-length proteins (Compare FIG. 22 and FIG. 26C). These results indicate that the T-loop peptides can be employed as bona fide substrates to measure LKB1 activity in a facile single step assay. The NUAK2 T-loop peptide was termed LKBtide.

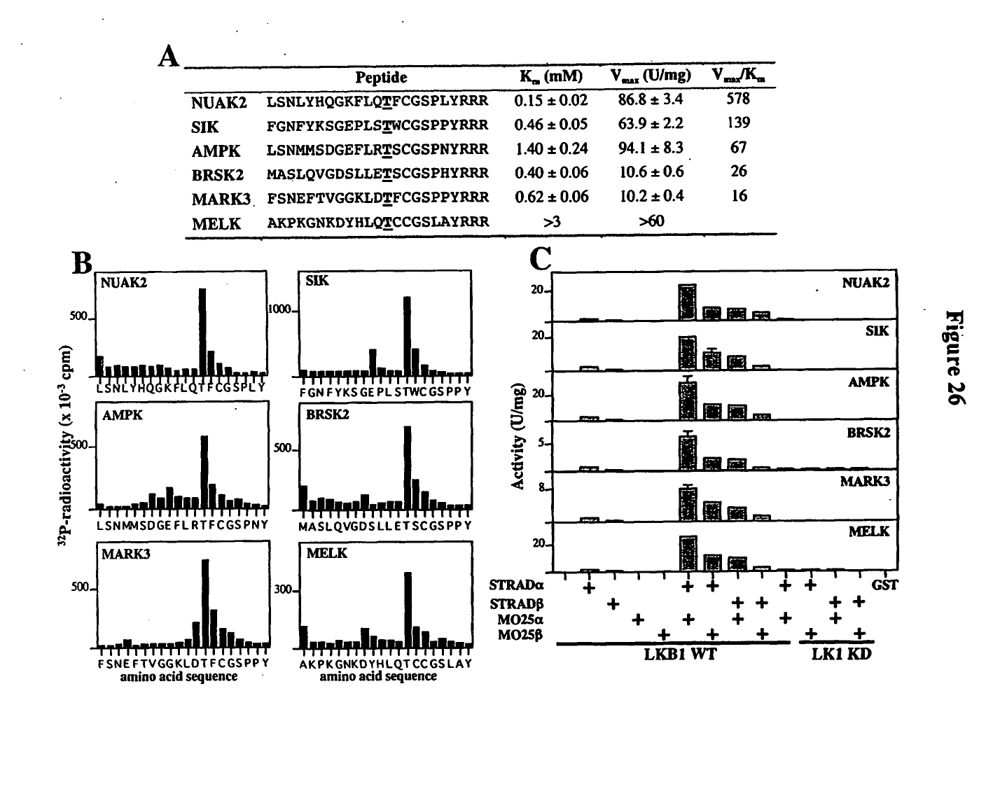

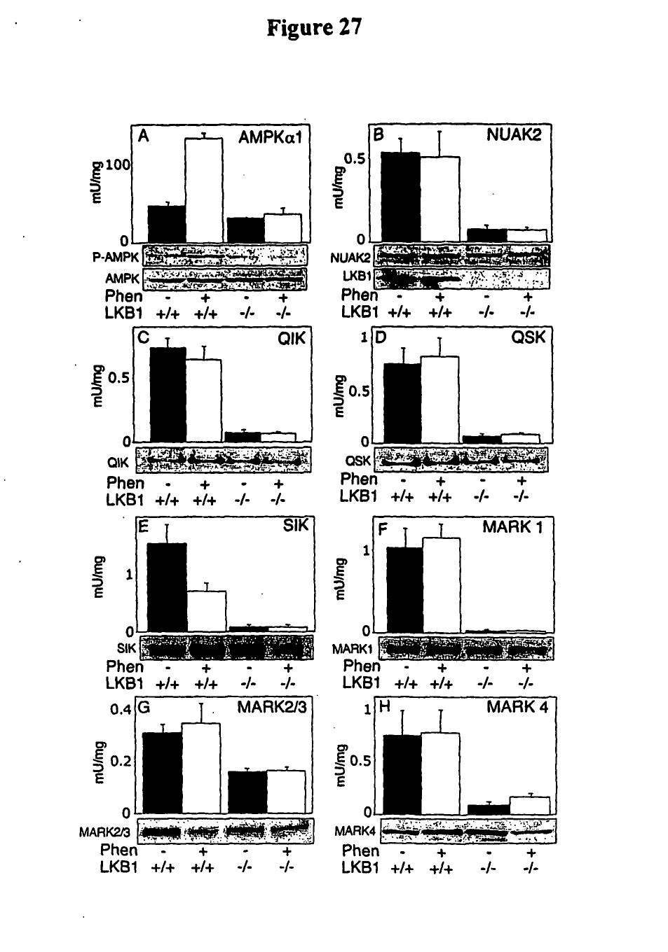

Role of LKB1 in regulating AMPK-related kinases in fibroblasts. In order to investigate the role of LKB1 in regulating AMPK-related kinases in intact cells, we generated peptide antibodies against specific sequences in the twelve AMPK-related kinases. The ability of these antibodies to immunoprecipitate the active forms of the AMPK-related kinases was assessed employing HEK-293 cell lysates in which the kinases had been over-expressed and found to be active (data not shown). Using this approach we were able to develop antibodies that selectively immunoprecipitated all the AMPK-related kinases except for MARK and MARK3, whose activity was assessed using a commercial antibody that immunoprecipitated both of these kinases, but not MARK1 and MARK4 (data not shown). Using the specific antibodies, we were able to immunoprecipitate and assay endogenously expressed NUAK2, QIK, QSK, SIK MARK1, MARK2/3 and MARK4 in LKB1^{+/+} mouse embryonic fibroblasts (MEFs) (FIG. 27). We then compared the activities of these kinases immunoprecipitated from LKB1^{+/+} and LKB1^{-/-} MEFs, and found that the activity of NUAK2, QIK, QSK, SIK, MARK1 and MARK4 was reduced between 7 to 35-fold in the LKB1^{-/-} MEFs (FIG. 27). The combined MARK2/3 activity was reduced ˜3-fold in the LKB1−/− MEFs (FIG. 27G). Immunoblotting demonstrated that the reduced activity of AMPK-related kinases in LKB1^{-/-} cells was not caused by a decrease in expression of the enzymes, which were either present at the same or slightly reduced levels in the knockout cells.

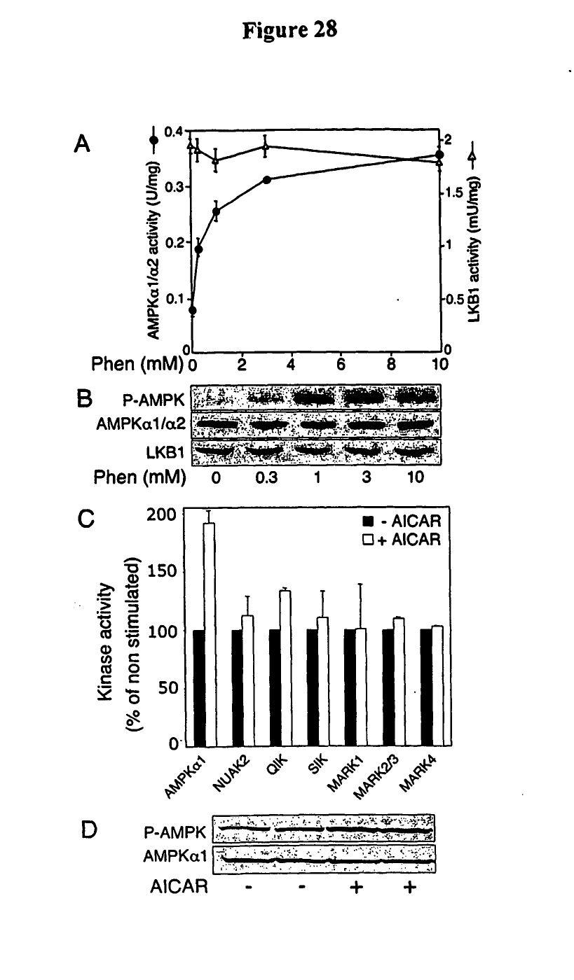

Interestingly, and in contrast to AMPKα1 (FIG. 27A), the AMPK-related kinases assayed, were not significantly stimulated phenformin (FIG. 27B to 27H). This suggested that phenformin was not triggering activation of AMPK by activating LKB1. Consistent with this, we demonstrated that increasing doses of phenformin that progressively activated AMPK (and increased the phosphorylation of Thr172), but did not stimulate LKB1 activity (FIG. 28A). The drug 5-aminoimidazole-4-carboxamide (AICA) riboside activates AMPK in intact cells by being taken up and converted by adenosine kinase to AICA riboside monophosphate, which mimics the effect of AMP on the AMPK system (Corton et al., 1995). AICA riboside activated AMPK in LKB1^{+/+} MEFs, but failed to markedly activate any of the AMPK-related kinases (FIG. 28B).

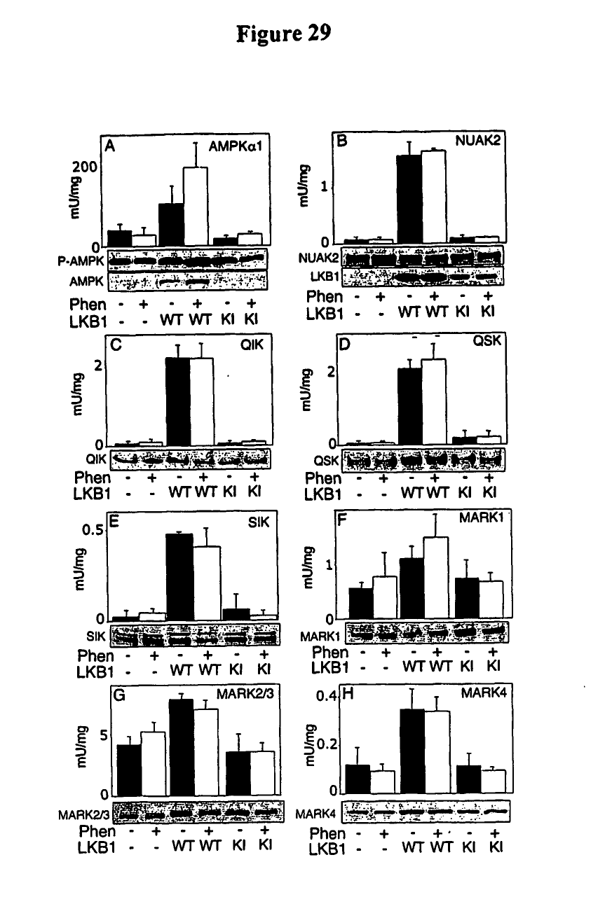

Expression of LKB1 restores activation of AMPK-related kinases in HeLa cells. To obtain further genetic evidence that LKB1 acts as an upstream regulator of the AMPK-related kinases, we compared the activity of AMPK-related kinases in HeLa cells, which do not to express LKB1, with HeLa cells stably expressing either wild type or catalytically inactive LKB1 (Example 2; Hawley et al., 2003; Sapkota et al., 2002). In control HeLa cells not expressing LKB1, or cells expressing catalytically inactive LKB1, the activity of NUAK2, QIK, QSK and SIK was 20 to 40 fold lower than that observed in HeLa cells that express wild type LKB1 (FIG. 29B to 29E). MARK1, combined MARK2/3 activity and MARK4 activity in control HeLa cells was about ˜2-3 fold lower than in cells expressing LKB1 (FIG. 29F to 29H). In contrast to AMPKα1 (FIG. 29A), none of the AMPK-related kinases were significantly activated when HeLa cells were stimulated with phenformin (FIG. 29B to 29H).

Discussion

In this Example we provide evidence that LKB1 functions to regulate the activity of 11 of the 12 members of the AMPK-related family of protein kinases. In cell-free systems we established that all AMPK-related kinases tested, with the exception of MELK, are activated over 50-fold following the phosphorylation of their T-loop Thr residue, by the LKB1:STRAD:MO25 complex. In the case of MELK, our results indicate that this enzyme requires T-loop phosphorylation for activity, but that it is able to phosphorylate its own T-loop residue. To our knowledge this is the first demonstration that the BRSK1, BRSK2, NUAK1, NUAK2, QIK, QSK, SIK as well as MELK enzymes can be activated by phosphorylation. Mutation of the T-loop T1 residue to Ala prevented the activation of all of these enzymes by the LKB1 complex, whereas its mutation to Glu was sufficient to activate BRSK1, BRSK2, NUAK1, NUAK2, SIK, QIK, QSK and MARK4 to specific activities similar to that observed for the LKB1-phosphorylated forms of these enzymes (FIG. 24). This latter finding could be exploited in future over-expression or knock-in studies to introduce constitutively active forms of these enzymes in cells to examine their cellular roles.

Previous work has indicated that MARK2 and MARK3 were phosphorylated at the T-loop Thr residue as well as at a nearby Ser residue (Drewes et al., 1997; Johnson et al., 1996). The T-loop Ser residue phosphorylated in MARK2/MARK3 is conserved in AMPKα1, AMPKα2 and all AMPK-related kinases (FIGS. 21A & B), but at least for AMPKα1 there is no evidence that this residue is phosphorylated in vivo (Woods et al., 2003b). Our analysis (FIGS. 25A and 25B), as well as that of a recent study (Timm et al., 2003), showed that phosphorylation of the T-loop Thr residue of the MARK kinases, played the most crucial role in regulating the activity of this class of kinase. The LKB1 complex phosphorylated a catalytically inactive MARK3 mutant at both the Thr and Ser T-loop residue (FIG. 25A). However, the isolated MARK3 T-loop peptide was only phosphorylated by LKB1 at the Thr residue (FIG. 26), signifying that the primary sequence of the peptide was not sufficient to enable LKB1 to phosphorylate the Ser residue. The finding that mutation of the T-loop Thr211 to Ala on MARK3 prevented phosphorylation of Ser215 by the LKB1 complex, suggests that phosphorylation of Thr211 is required for Ser215 phosphorylation. MARK4 may be regulated differently as peptide-mapping studies indicated that MARK4 was only phosphorylated at the T-loop Thr by LKB1 (FIG. 25G), and that mutation of the T-loop Thr to Glu increased activity to a much greater level than observed for other MARK isoforms (FIG. 25F). It was previously reported for MARK2 that mutation of the T-loop to Glu increased its specific activity 3-fold (Timm et al., 2003) and we have made similar findings with this mutation increasing MARK2 activity form 2.2 U/mg to 6.7 U/mg (FIG. 25D). However, in contrast to TAO1 that only activated MARK2 10-fold (Timm et al., 2003), the LKB1 complex increased MARK2 activation 200-fold, establishing that mutation of the MARK2 T-loop Thr to Glu does not markedly activate the kinase.

As observed for the activation of AMPKα1 and AMPKα2 (Hawley et al., 2003), the presence of STRAD and MO25 subunits was essential for LKB1 to activate all the AMPK-related kinases in cell free-assays. There is considerable evidence that many protein kinases rely on residues, termed docking sites, lying outside of the catalytic core, to bring the kinase and substrate together. It is possible that the STRAD and MO25 subunits play a role in facilitating the interaction with, and hence the phosphorylation by LKB1. The finding that the LKB1:STRAD:MO25 complex phosphorylated the short T-loop peptide of AMPKα1 and AMPK-related kinases, at a much higher rate than LKB1 alone, indicated that the STRAD and MO25 subunits activate LKB1 directly. However, this result does not rule out the possibility that STRAD and MO25 also play roles in substrate recognition.

Employing LKB1^{-/-} MEFs and/or HeLa cells that lack LKB1, we were able to demonstrate that the activities of endogenously expressed NUAK1, NUAK2, QIK, QSK, and SIK were 7 to 40-fold lower than in LKB^{+/+} MEFs or HeLa cells that stably express wild type LKB1. This provides genetic evidence that LKB1 is rate-limiting in activation of these enzymes in intact cells. However, our data do not rule out the possibility that other kinases can regulate the activity of the AMPK-related kinases in vivo in addition to LKB1. Recently the TAO1 kinase was purified from pig brain as the major activity that phosphorylated the T-loop Thr residue of MARK2, resulting in its activation (Timm et al., 2003). Although TAO1 also activated MARK2 in over-expression studies (Timm et al., 2003), thus far no genetic evidence indicating how the lack of TAO1 affected the activity of MARK family kinases in vivo, has been reported. Such evidence may be hard to acquire, as mammalian cells possess 3 closely related TAO isoforms (Manning et al., 2002). Lack of specific immunoprecipitating antibodies meant that we were unable to establish how a deficiency of LKB1 affected the individual activities of MARK2 or MARK3. However, the finding that the combined activity of MARK2 and MARK3 in LKB1^{-/-} and control HeLa cells was substantial, implies that other kinases, such as TAO1, may regulate the activities of these enzymes. It should be noted that LKB1 and TAO1 share no obvious amino acid sequence homology and lie in distinct regions of the human kinase dendrogram (Manning et al., 2002). TAO1 belongs to the STE20 group of kinases and is therefore related to STRADα and STRADβ, but whether this is significant is not known.

The MARK (MAP/microtubule affinity regulating kinase) family of kinases are the most studied AMPK-related kinase family members and are thought to play key roles in establishing cell polarity. For example genetic studies indicate that MARK homologues control partitioning of the C. elegans zygote (Guo and Kemphues, 1995) and embryonic axis formation in Drosophila (Shulman et al., 2000; Tomancak et al., 2000). In neuronal cells, MARK kinases phosphorylate the neuronal microtubule associated protein Tau, resulting in the destabilisation of microtubules (Drewes et al., 1997). Interestingly, the counterpart of mammalian LKB1 in C. elegans (Watts et al., 2000), termed PAR4, was originally identified as a member of the maternally expressed PAR (partitioning defective) gene family, required for establishing cell polarity during the first cycle of C. elegans embryogenesis (Kemphues et al., 1988). Maternal lethal mutations in the gene encoding C elegans PAR4, have been shown to affect several aspects of cell polarity (Morton et al., 1992). These lead to phenotypes similar to those observed in C elegans mutated in the PAR1 gene, which encodes the homologue of MARK3 (Guo and Kemphues, 1995). More recently, both the Drosophila (Martin and St Johnston, 2003) and Xenopus (Ossipova et al., 2003) homologues of human LKB1 were shown to play important roles in regulating cell polarity. Originally, it was suggested that Drosophila LKB1 functioned downstream of PAR1/MARK3, as over-expression of Drosophila LKB1 suppressed the polarity phenotype of PAR1/MARK3 mutants (Martin and St Johnston, 2003). However, in HeLa cells that do not express LKB1, MARK3 was found to exist in a dephosphorylated state and reintroduction of LKB1 promoted phosphorylation of MARK3 at its T-loop (Spicer et al., 2003), signifying that LKB1 was upstream of MARK3. One explanation that would reconcile the apparent discrepancy between the Drosophila and mammalian studies would be if over-expression of Drosophila LKB1 suppressed the polarity phenotype of PAR1/MARK3 mutants, through the activation of other AMPK-related kinases, which might be able to compensate for the loss of PAR1/MARK3 function.

Much less is known regarding the function of other AMPK-related kinases, i.e. BRSK1, BRSK2, NUAK1, NUAK2, QIK, QSK and SIK. Previous Northern blotting of NUAK1, NUAK2, QIK and SIK performed by other groups (see below) and analysis of EST clones (Table 2), indicated that the AMPK-related kinases with the exception of BRSK1, BRSK2 and MELK are likely to be expressed in many tissues. BRSK1 and BRSK2 EST clones were mainly derived from neuronal tissues and, after immunoblot analysis of a variety of rat tissues, these enzymes were only detected in brain and at low levels in testis (K. Sakamoto, unpublished results). To date, MELK (maternal embryonic leucine zipper kinase) has only been reported to be expressed during mammalian embryogenesis, with the strongest expression detected during maturation of oocytes and preimplantation development (Heyer et al., 1999; Heyer et al., 1997). Although our antibodies raised against BRSK1, BRSK2 and MELK readily immunoprecipitated the recombinant enzymes, we were unable to detect their activity in MEFs or HeLa cells (OG, data not shown). SIK (Salt-inducible kinase) was first cloned from the adrenal glands of rats fed a high salt diet (Wang et al., 1999). SIK mRNA was also induced by membrane depolarization in brain (Feldman et al., 2000) and recent studies have indicated that when SIK is over-expressed in cells, it might play a role in steroidogenesis (Takemori et al., 2003). The mRNA expressing QIK (also termed SIK2), was highest in adipose tissue and, in over-expression studies, QIK was reported to phosphorylate human IRS1 at Ser794 (Horike et al., 2003), the residue equivalent to Ser789 in rat IRS1, shown to be phosphorylated by AMPK (Jakobsen et al., 2001). In other over-expression studies, NUAK1 (ARK5) was shown to suppress apoptosis induced by some stimuli, including nutrient starvation (Suzuki et al., 2003a). Furthermore, it has been claimed that Akt/PKB phosphorylated NUAK1 at a C-terminal site outside of the catalytic domain, leading to a 3-fold activation of the enzyme (Suzuki et al., 2003b). NUAK2 (SNARK) was most highly expressed in the kidney, and its activity reportedly stimulated by glucose starvation of cells (Lefebvre et

Supplementary Table. Tissue distributions of ESTs encoding the
indicated AMPK-related kinases, Abbreviation No., number

| | |
|---|---|
| Tissue | No. |

BRSK1

| | |
|---|---|
| Brain | 1 |
| Nervous System | 6 |
| [symbol] of [symbol] | 3 |
| Lung tumour | 3 |
| Heart | 2 |

Other ( [symbol] , fetal eye, [symbol] ) 1 per tissue type

BKSK2

| | |
|---|---|
| Eye | 14 |
| Nervous system | 13 |
| Breast | 10 |
| Brain | 8 |
| [symbol] cells | 3 |
| Var. [symbol] cells | 7 |

NUAK1

| | |
|---|---|
| [symbol] | 18 |
| [symbol] | 7 |
| Liver/spleen | 5 |
| Eye | 4 |
| Uterus | 4 |
| Prostate | 3 |
| Var. tumour cells | 9 |

Other (lung, head/neck, breast, adipose tissue, placenta, [symbol]
cells) less than 3 per tissue type.

NUAK2

| | |
|---|---|
| Blood | 18 |
| Placenta | 6 |
| Brain | 6 |
| Ovary | 6 |
| Nervous system | 3 |
| Kidney | 2 |
| Colon | 2 |
| Eye | 2 |
| Var. tumour cells | 10 |

Other ( [symbol] , head/neck, cervix, lung)
1 per tissue type.

MELK

| Blood | 7 |
|---|---|
| Adenocarcinoma | 7 |
| Stomach | 5 |
| [symbol] B cell | 5 |
| [symbol] cell [symbol] | 4 |
| [symbol] | 4 |
| Other(cervix, eye, nervous system, intestine, heart, | |
| [symbol] , uterus, placenta) less than 3 per tissue type. | |
| QIK | |
| Eye | 17 |
| Liver/spleen | 12 |
| Blood | 14 |
| Brain | 13 |
| Breast | 10 |
| Intestine | 11 |
| Intestinal tumour | 10 |
| [symbol] | 9 |
| Nervous system | 9 |
| [symbol] | 7 |
| Lung | 7 |
| Placenta | 7 |
| Heart | 6 |
| Fibroblast | 6 |
| Kidney | 5 |
| Germ cell tumour | 5 |

Other (prostate, uterus, testis, head, pancreas,
bone, stomach) less than 5 per tissue type.

SIK

| | |
|---|---|
| Uterus | 12 |
| Adenocarcinoma | 12 |
| Intestine | 12 |
| Brain tumour | 11 |
| Breast | 8 |
| Kidney | 8 |
| Adenocyte | 7 |
| [symbol] | 7 |
| Stomach | 6 |
| Brain | 5 |
| Fetal heart | S |
| Heart | s |
| Head/neck | s |

Other (placenta, cervical tumour, [symbol] , [symbol] , lung, prostate,
marrow, eye, bladder tumour) less than 5 per tissue type.

QSK

| | |
|---|---|
| Liver/spleen | 25 |
| Lung carcinoma | 23 |
| [symbol] | 14 |
| Head/neck | 13 |
| Nervous syst. [symbol] | 11 |
| Adenocarcinoma | 10 |
| Placenta | 10 |
| [symbol] | 8 |
| Pancreas | 8 |
| Eye | 7 |
| Carcinoid | 7 |
| Heart | 6 |
| Kidney | 6 |

Other (breast, intestine, testis, lung, uterus, heart)
less than 6 per tissue type.

MARK1

| | |
|---|---|
| Brain | 17 |
| Carcinoid | 12 |
| Breast | 7 |
| Lung tumour | 6 |
| Pancreas | 5 |
| Eye | 4 |

Other (heart head/neck kidney, [symbol] , placenta)
less than 3 per tissue type.

MARK2

| | |
|---|---|
| Head/neck | 18 |
| Brain | 17 |
| Intestine | 13 |
| Adenocarcinoma [symbol] | 11 |
| Pancreas | 9 |
| Placenta | 8 |
| Eye | 8 |
| Blood | 7 |
| Melanoma | 6 |

Other (prostate, kidney, lung, bone, uterus, [symbol]
, nervous system)
less than 6 per tissue type.

MAKK3

| | |
|---|---|
| Brain | 27 |
| Nervous system | 26 |
| Liver/spleen | 23 |
| Eye | 21 |
| Placenta | 19 |
| Lung | 17 |
| Colon | 8 |
| Stomach | 7 |
| Heart | 6 |
| Uterus | 6 |
| Kidney | 6 |
| Skeletal muscle | 5 |
| Cervix | 5 |
| Blood | 5 |
| Var. tumour cells | 102 |

Other (prostate, testis, [symbol] , lymph, marrow,
breast) less than 5 per tissue type.

MARK4

| | |
|---|---|
| Brain | 13 |
| Eye | 13 |
| [symbol] tissue | 11 |
| [symbol] | 7 |
| Colon | 4 |
| Stomach | 3 |
| Breast | 3 |
| Prostate | 3 |
| Testis | 2 |
| Lung | 2 |
| Var. tumour cells | 43 |

al., 2001; Suzuki et al., 2003b). To our knowledge, no previous studies have addressed the roles of BRSK1, BRSK2 and QSK.

Clearly, further work needs to be carried out to further characterise the mechanism of regulation and function of the AMPK-related kinases. Our studies indicate that, at least in MEFs and HeLa cells that stably express LKB1, the AMPK-related kinases were not activated in response to the drugs phenformin or AICA-riboside, in contrast to AMPK. This suggests that the beneficial anti-diabetic effects of phenformin and metformin may be mediated through the activation of AMPK rather than the AMPK-related kinases. The finding that AMPK-related kinases are not activated by phenformin or AICA riboside, is consistent with our findings that these treatments do not activate LKB1 directly in LKB1^{+/+} MEFs (FIG. 28A) or in COS7 cells (Woods et al., 2003a). In future studies it will be important to define the physiological stimuli that do cause activation of the AMPK-related kinases, and establish whether these enzymes, like AMPK, possess regulatory subunits that control their activation.

A significant number of inherited forms of PJS found in certain families do not display mutations in the LKB1 gene (Buchet-Poyau et al., 2002; Resta et al., 2002), indicating that there is a second causative locus for PJS. It will clearly now be critical to find out whether PJS families that express the normal LKB1 protein have mutations in other AMPK-related kinases.

In conclusion, we have shown that LKB1 functions as a master upstream protein kinase activating 11 AMPK-related kinases in addition to AMPKα1 and AMPKα2. It is possible that these enzymes mediate some of the physiological effects previously ascribed to LKB1 and that one or more of these kinases may themselves function as tumour suppressors.

Materials. Protease-inhibitor cocktail tablets and sequencing grade trypsin were obtained from Roche. N-octyl-glucoside was from Calbiochem, and tissue culture reagents were from Life Technologies. Tetracyclin-free foetal bovine sera (FBS) was from Perbio, and other tissue culture reagents were from Biowhittaker. Precast 4-12% and 10% polyacrylamide Bis-Tris gels were obtained from Invitrogen; P81 phosphocellulose paper was from Whatman; Phenformin from Sigma and AICA riboside from TorontoXX. [γ-³²P]ATP, glutathione-Sepharose, Protein G-Sepharose were purchased from Amersham Biosciences. All peptides were synthesised by Dr Graham Bloomberg at the University of Bristol.

Antibodies.

The following antibodies were raised in sheep and affinity purified on the appropriate peptide antigen: BRSK1 (MVAGLTLGKGPESPDGDVS, residues 1-20 of human BRSK1), BRSK2 (LSWGAGLKGQKVATSYESSL, residues 655-674 of human BRSK2), NUAK1 (MEGAAAPVAGDRPDLGLGAPG residues 1-21 of human NUAK1), NUAK2 (TDCQEVTATYRQALRVCSKLT, residues 653-673 of human NUAK2), QIK (MVMADGPRHLQRGPVRVGFYD, residues 1-21 of human QIK), SIK (MVIMSEFSADPAGQGQGQQK, residues 1-20 of human SIK used for immunoprecipitation from human cells), SIK (GDCEMEDLMPCSLGTFVLVQ, residues 765-784 of human SIK used for immunoprecipitation from mouse cells), QSK (TDILLSYKHPEVSFSMEQAGV, residues 1349-1369 of human QSK), MARK1 (SGTSIAFKNIASKIANELKL, residues 776-795 of human MARK1), MARK4 (MSSRTVLAPGNDRNSDTHGT, residues 1-20 of human MARK4), MELK (MKDYDELLKYYELHETIGTG, residues 1-20 of human MELK). The specific AMPKα1) antibody employed in FIGS. 27, 28B and 29 was raised against the peptide (CTSPPDSFLDDHHLTR, residues 344-358 of rat AMPKα1) whilst the antibody recognising both AMPKα1 and AMPKα2 employed in FIG. 28A, was raised against the peptide (CDPMKRATIKDIRE residues 252 to 264 or rat AMPKα1). The anti MARK2 and MARK3 peptide antibodies we raised were not specific as they immunoprecipitated other MARK isoforms as well. The anti MARK3 antibody from Upstate Biotech (anti c-TAK #05-680), raised against the human MARK3 protein was found to immunoprecipitate MARK2 as efficiently as MARK3 but did not immunoprecipitate MARK1 and MARK4 (data not shown). The Phosphospecific antibodies recognising AMPK phosphorylated on the T-loop were generated against as described previously against the peptide (KFLRT(P)SCGSPNYA residues 168 to 180 of rat AMPKα1) (Sugden et al., 1999). The LKB1 antibody used for immunoblotting was raised in sheep against the mouse LKB1 (Sapkota et al., 2001) and that for immunoprecipitation was raised in sheep against the human LKB1 protein (Boudeau et al., 2003). Monoclonal antibody recognizing the HA epitope tag was from Roche, monoclonal antibodies recognising the GST and the FLAG epitopes were from Sigma. Anti-myc antibodies were prepared by ammonium sulphate precipitation of medium from Myc1-9E10 hybridoma cells grown in RPMI 1640 medium supplemented with 2 mM glutamine and 15% (v/v) foetal bovine serum. Secondary antibodies coupled to horseradish peroxidase used for immunoblotting were obtained from Pierce.

General methods and buffers. Restriction enzyme digests, DNA ligations, and other recombinant DNA procedures were performed using standard protocols. All mutagenesis was performed using the QuikChange site-directed mutagenesis method (Stratagene). DNA constructs used for transfection were purified from E. coli DH5α using Qiagen plasmid Mega kit according to the manufacturer's protocol. All DNA constructs were verified by DNA sequencing, which was performed by The Sequencing Service, School of Life Sciences, University of Dundee, Scotland, UK, using DYEnamic ET terminator chemistry (Amersham Biosciences) on Applied Biosystems automated DNA sequencers. Lysis Buffer contained 50 mM Tris/HCl pH 7.5, 1 mM EGTA, 1 mM EDTA, 1% (w/v) Triton-X 100, 1 mM sodium orthovanadate, 50 mM sodium fluoride, 5 mM sodium pyrophosphate, 0.27 M sucrose, 0.1% (v/v) 2-mercaptoethanol and ‘complete’ proteinase inhibitor cocktail (one tablet/50 ml). Buffer A contained 50 mM Tris/HCl pH 7.5, 0.1 mM EGTA, and 0.1% (v/v) 2-mercaptoethanol. SDS sample Buffer contained 50 mM Tris/HCl pH 6.8, 2% (w/v) SDS, 10% (v/v) glycerol, 0.005% (w/v) bromophenol blue, and 1% (v/v) 2-mercaptoethanol.

Cloning of AMPK-Related Kinases.

NUAK1. In order to obtain the coding region of human NUAK1 cDNA (NCBI Acc. NM_{—}014840) a PCR reaction was carried out using a brain cDNA library (Clontech) as a template and the sense primer 5′-actgcagccctggagcccaggaagc-3′ and the antisense primer 5′-ctagttgagcttgctgcagatctccagcgc-3′. The resulting PCR product covered the coding region of NUAK1 from amino acid residues 31-661. The cDNA of the missing N-terminal 31 amino acids was incorporated into the 5′ primer of another PCR reaction, then the HA tag was added by PCR using the sense primer 5′-actagtgccaccatgtacccatacgatgtgccagattacgccgaaggggccgccgcgcctgtggcgggg-3′ and antisense primer 5-ctagttgagcttgctgcagatctccagcgc-3′. The resulting PCR product was ligated into pCR2.1-TOPO vector (Invitrogen), and subcloned as a Spe1-Not1 fragment into pEBG2T (Sanchez et al., 1994) and as an EcoR1-EcoR1 insert into pGEX6P-1 (Amersham) expression vectors.

NUAK2. The coding region of human NUAK2 cDNA (NCBI Acc. NP_{—}112214) with an N-terminal HA tag, was amplified by PCR from IMAGE consortium EST clone 6718982 (NCBI Acc. CA487493) with primers actagtgccaccatgtacccatacgatgtgccagattacgccgagtcgctggttttcgcgcggcgctcc⁻³′ and 5′-tcaggtgagctttgagcagaccctcagtgcctg-3′. The resulting PCR product was ligated into pCR2.1-TOPO vector, sequenced, and subcloned as a Spe1-Spe1 fragment into pEBG2T and as an EcoR1-EcoR1 insert into pGEX6P-1 expression vectors.

QIK. The coding region of human QIK cDNA (NCBI Ace. XM_{—}041314) with an N-terminal HA tag was amplified from IMAGE consortium EST clone 5495545 (NCBI Ace. BM799630) using sense primers 5′-gcgtcgactacccatacgatgtgccagattacgccgtcatggcggatggcccgag-3′ or 5′-gcactagttacccatacgatgtgccagattacgccgtcatggcggatggcccgag-3′ for subsequent cloning into vectors pGEX6P-3 and pEBG2T respectively, and antisense primer 5′-gagcggccgctaattcaccaggacatacccgttgtg-3′. Amplified PCR products were ligated into pCR2.1 TOPO vector, sequenced, and subcloned as a Sal1-Not1 or Spe1-Not1 insert into pGEX6P-3 or pEBG2T respectively.

SIK. The coding region of human SIK cDNA (NCBI Acc. NM_{—}173354) with an N-terminal HA tag was amplified from IMAGE consortium EST clone 4831049 (NCBI Acc. NM_{—}173354) using primers 5′-gcggatcctacccatacgatgtgccagattacgccgttatcatgtcggagttcagcgcgg-3′ and 5-gagcggccgctcactgcaccaggacaaacgtgcc-3′. The PCR product was ligated into pCR2.1 TOPO vector, sequenced, and subcloned as a BamH1-Not1 insert into pGEX6P-1 and pEBG2T.

MELK. The coding region of human MELK cDNA (NCBI Acc. NM_{—}014791) with an N-terminal HA tag was amplified from IMAGE consortium EST clone 4547136 (NCBI Acc. BC014039) using primers 5′-gcggatcctacccatacgatgtgccagattacgccaaagattatgatgaacttctcaaatattatgaattacatg-3′ and 5′-gtgcggccgcttataccttgcagctagataggatgtcttcc-3′. The PCR product was ligated into pCR2.1 TOPO vector, sequenced, and subcloned as a BamH1-Not1 insert into pGEX6P-1 and pEBG2T.

BRSK1. Nucleotides 60-1010 of the coding region of human BRSK1 cDNA (NCBI Acc. NM_{—}032430) were amplified from IMAGE consortium clone 6154749 (NCBI Acc. BQ434571) using primers 1. 5′-ccacccccacccaccccagcacgcccaatatgtgggcccctatcggctggagaagacgctgggcaaagg-3′ and 2. 5′-cgatgcagcctctcgcggtccctgaagcagc-3′, nucleotides 60-106 being added by primer 1. Nucleotides 980-2337 of BRSK1 were amplified from the same EST clone using primers 3. 5′-gctgcttcagggaccgcgagaggctgcatcg-3′ and 4. 5′-tcagggcagaggggtcccgttggtggcc-3′. Each PCR product was ligated into pCR2.1 TOPO vector and sequenced. A single nucleotide difference compared with BRSK1 NM_{—}032430 sequence that is present in the EST clone was ‘corrected’ by site-directed mutagenesis in the pCR2.1 TOPO clone containing the 5′ half of BRSK1. The remaining 5′ 59 nucleotides of BRSK1 and the HA tag were added to the 5′ half of BRSK1 by PCR using sense primers 5. 5′-ggtgggggctctcccgcctaccacctcccccacccccacccccacccaccccagcacgcccaatatg-3′ and 6. 5′-ggatcctacccatacgatgtgccagattacgcctcgtccggggccaaggagggaggtgggggctctcccg cctacc-3′. The final PCR product was ligated into pCR2.1 TOPO vector and sequenced. Overlap PCR was then performed using the 5′ half and 3′ half of BRSK1 as templates and primer 7. 5′-gcggatcctacccatacgatgtgcc-3′ and primer 4. and the PCR product ligated into pCR2.1 TOPO vector and sequenced. Full length BRSK1 cDNA with N-terminal HA tag was then subcloned into pGEX6P-1 and pEBG2T as a BamH1-BamH1 insert.

BRSK2. The coding region of HA-tagged human BRSK2 cDNA (NCBI Ace. AF533878) was amplified by PCR from IMAGE consortium EST clone 6144640 (NCBI Acc. BU193218) using primers 5′-ggatccgccaccatgtacccatacgatgtgccagattacgccacatcgacggggaaggacggcggcgcg-3′ and 5′-gcggccgctcagaggctactctcgtagctggtggccaccttctggcccttaagccca-3′. The resulting PCR product was cloned into pCR2.1-TOPO vector, sequenced, and subcloned as a BamH1-Not1 insert into pEBG2T and pGEX6P-1 vectors.

QSK. Nucleotides 55-640 of the coding region of human QSK cDNA (sequence obtained from Sugen database www.kinase.com (Manning et al., 2002)) were amplified from IMAGE consortium EST clone 4396995 (NCBI Acc.BF983268) using primers 1. 5′-ggagccgggcccgcgggccgcctgctgcctccgcccgcgccggggtccccagccgcccccgctgccgt gtcccctgcggccggccagccg-3′ and 2. 5′-tgaagaggttactgaaaccaaaatctgctattttgatattc-3′, nucleotides 55-110 being added by primer 1. The remaining 5′ 54 nucleotides and HA tag were subsequently added by PCR using primers 3. 5′gattacgccgcggcggcggcggcgagcggagctggcggggctgccggggccgggactgggggagcc gggcccgcgggccgcctgctg-3′ and 4. 5′-gcggatcctacccatacgatgtgccagattacgccgcggcggcggcggcgagcgg-3′. The final PCR product was ligated into pCR2.1 TOPO vector and sequenced to produce pCR2.1 QSK clone 1. Nucleotides 610-865 of QSK were amplified from IMAGE consortium EST clone 6247938 (NCBI Acc. BQ685213) using primers 5. 5′-atagcagattttggtttcagtaacctcttcactcctgggcagctgctgaagacctggtgtggcagccctcccta tgctgcacctgaactc-3′ and 6. 5′-ctgtggacataaaaaatgggatgcggaactttcc-3′, nucleotides 610-674 being added by primer 5. The PCR product was ligated into pCR2.1 TOPO vector and sequenced. A single nucleotide deletion at QSK open reading frame position 696, which is present in the EST clone, was corrected by site-directed mutagenesis to produce pCR2.1 QSK clone 2. QSK PCR products from pCR2.1 QSK clones 1 and 2 were input into an overlap PCR using primers 4. and 6. This product (QSK nucleotides 4-865 with N-terminal HA tag) was ligated into pCR2.1 TOPO vector and sequenced to produce pCR2.1 QSK clone 3.

QSK nucleotides 832-4110 were amplified from IMAGE consortium EST clone 360441 (NCBI Acc. AA015726) using primers 7. 5′-ggaaagttccgcatcccattttttatgtccacag-3′ and 8. 5′-gagcggccgcttacacgcctgcctgctccatgc-3′. The PCR product was ligated into pCR2.1 TOPO vector and sequenced to produce pCR2.1 QSK clone 4.

QSK PCR products from pCR2.1 clones 3 and 4 were input into an overlap PCR using primers 4. and 8. to generate full length QSK cDNA with N-terminal HA tag. The PCR product was ligated into pCR2.1 TOPO vector and sequenced to produce pCR2.1 QSK clone 5. Full length QSK with N-terminal HA tag was subcloned from pCR2.1 QSK clone 5 as a BamH1-BamH1 insert into pGEX6P-1 and pEBG2T vectors. In order to generate the T-loop mutations in QSK, mutagenesis was performed on pCR2.1 QSK clone 3, followed by overlap PCR to generate the full length mutant forms of QSK. The overlap PCR product was ligated into pCR2.1 TOPO, sequenced, and subcloned into pGEX6P-1 and pEBG2T as a BamH1-BamH1 insert.

MARK1. Nucleotides 4-1078 of the coding region of human MARK1 cDNA (NCBI Acc. NM_{—}018650) with an N-terminal HA tag were amplified from a human brain cDNA library using primers 5′-gcgaattctacccatacgatgtgccagattacgcctcggcccggacgccattgc-3′ and 5′-catcatacttctgatttattaaggcatcatttatttc-3′. Nucleotides 1042-2388 of MARK1 were amplified from IMAGE consortium EST clone 48109 (NCBI Acc. H11850) using primers 5′-gaaataaatgatgccttaataaatcagaagtatgatg-3′ and 5′-gagtcgacttacagcttaagctcatttgctatttttgatgc-3′. Amplified PCR products were input into an overlap PCR to generate the full length HA-tagged MARK1 cDNA. The PCR product was ligated into pCR2.1 TOPO vector, sequenced, and subcloned as an EcoR1-Sal1 insert into pGEX6P-1. Full length HA-tagged MARK1 was amplified from the pCR2.1 TOPO MARK1 clone for subsequent cloning into pEBG2T as a Kpn1-Not1 insert using primers 5′-gcggtacctacccatacgatgtgccagattacgcctcggcccggacgccattgc-3′ and 5′-gagcggccgcttacagcttaagctcatttgctatttttgatgc-3′.

MARK2. The coding region of human MARK2 cDNA (NCBI Acc. NM_{—}004954) was amplified from IMAGE consortium EST clone 4591688 (NCBI Acc. BG419875) using sense primers 5′-gcgtcgactacccatacgatgtgccagattacgccattcggggccgcaactcagec-3′ or 5′-gcactagttacccatacgatgtgccagattacgccattcggggccgcaactcagcc-3′ for subsequent cloning into vectors pGEX6P-3 and pEBG2T respectively, and antisense primer 5′-gagcggccgcttaaagcttcagctcgttggctattttgg-3′. Amplified PCR products were ligated into pCR2.1 TOPO vector, sequenced, and subcloned as a Sal1-Not1 or Spe1-Not1 insert into pGEX6P-3 or pEBG2T vectors respectively.

MARK3. The coding region of human MARK3 cDNA (NCBI Acc. NM_{—}002376) with an N-terminal HA tag was amplified from IMAGE EST 5104460 (NCBI Acc. BI223323) using primers 5′-gcggccgcagccaccatgtacccatacgatgtgccagattacgcctccactaggaccccattgccaacggt ga-3′ and 5′-gcggccgcttacagctttagctcattggcaattttggaagc-3′. The resulting PCR product was cloned into pCR2.1-TOPO vector, sequenced, and subcloned as a Not1-Not1 insert into pEBG2T and pGEX6P-2 vectors.

MARK4. In order to obtain the full length coding region of human MARK4 cDNA (NCBI Acc.AK075272) two EST clones were used as templates for PCR reactions. The first 228 amino acids with an N-terminal HA tag was amplified from IMAGE consortium EST clone 6301902 (NCBI Acc.BQ709130) using primers 1. 5′-agatctgccaccatgtacccatacgatgtgccagattacgcctcttcgcggacggtgctggccccggg-3′ and 2. 5′-tgccctgaaacagctccggggcggc-3′. The remaining coding region was amplified from IMAGE consortium EST clone 5503281 (NCBI Acc. BM467107) with primers 3. 5′-gggatcgaagctggacacgttctgc-3′ and 4. 5′-gcggccgctcacactccaggggaatcggagcagccgggg-3. The resulting PCR products were used as templates in an overlap PCR reaction with primers 1. and 4. The PCR product was ligated into pCR2.1-TOPO, sequenced, then subcloned further as a Bgl2-Not1 insert into the BamH1-Not1 site of pEBG2T and pGEX6P-1 vectors.

Other DNA constructs. The DNA constructs encoding wild type GST-LKB1 or catalytically-inactive GST-LKB1 [D194A] in the pEBG-2T vector (Sapkota et al., 2001), FLAG-STRADα, FLAG-STRADβ, myc-MO25α, myc-MO25P in the pCMV5 vector (Example 1; Boudeau et al., 2003) or the kinase domain of AMPKα1[residues 1-308] in the pGEX vector (Scott et al., 2002) have been described previously.

Cell culture, stimulation and cell lysis. The generation of HeLa cells stably expressing wild type or kinase dead LKB1 has been described previously (Sapkota et al., 2002). The cells were cultured in Minimum Essential Medium Eagle supplemented with 10% (v/v) tetracycline-free-FBS, 1× non-essential amino acid solution, 1× penicillin/streptomycin solution (Invitrogen), 100 μg/ml zeocin and 5 μg/ml blasticidin (Invitrogen). The production and culture of immortalised LKB1^{+/+} and LKB^{-/-} MEF cells from E9.5 embryos, was described previously (Example 2; Hawley et al., 2003). Human kidney embryonic 293 cells were cultured in Dulbecco's modified Eagle's medium containing 10% FBS. Cells cultured on a 10 cm diameter dish in 10% (v/v) serum were left unstimulated, stimulated with 10 mM phenformin or 2 mM AICA riboside for 1 h and lysed in 1 ml of ice-cold Lysis Buffer after quick rinsing in PBS. MEF cell lysates were snap frozen in liquid nitrogen, thawed prior to use and centrifuged at 4° C. for 30 min at 23000×g to remove cell debris. HeLa cell lysates were centrifuged immediately at 4° C. for 15 min at 13 000 rpm. Protein concentrations were determined by the Bradford method with bovine serum albumin as the standard (Bradford, 1976).

Immunoblotting. Total cell lysates (10-50 μg) or immunoprecipitated protein (from 1 mg of cell lysate) were heated in SDS Sample Buffer, and subjected to SDS-PAGE and electrotransfer to nitrocellulose membranes. Membranes were then blocked in 50 mM Tris/HCl pH 7.5, 0.15M NaCl (TBS), 0.5% (by vol) Tween containing 10% (by mass) skimmed milk, and probed for 16 h at 4° C. in TBS, 0.5% (by vol) Tween, 5% (by mass) skimmed milk and 1 μg/ml of the indicated antibodies. Detection of proteins was performed using horse radish peroxidase conjugated secondary antibodies and the enhanced chemiluminescence reagent (Amersham Pharmacia Biotech, Little Chalfont, UK).

Expression and purification of AMPK-related kinases in E. coli. Unless otherwise indicated, pGEX constructs encoding full length wild type or mutant forms of GST-HA tagged AMPK-related kinases, or GST-AMPKα1 [1-308], were transformed into E. coli BL21 cells. One-litre cultures were grown at 37° C. in Luria broth containing 100 μg/ml ampicilin until the absorbance at 600 nm was 0.8. 100 μM isopropyl-β-D-galactoside was added, and the cells were cultured for a further 16 h at 26° C. For pGEX constructs encoding the wild type or mutant GST-HA-tagged QSK, induction of protein expression was carried out by culturing the cells until the absorbance at 600 nm was 1, and then 1 mM isopropyl-β-D-galactoside was added, and the cells cultured further for 1 h at 30° C. Pelleted cells were resuspended in 35 ml of ice-cold lysis buffer and lysed in one round of freeze/thawing, and the lysates were sonicated to fragment DNA. The lysates were centrifuged for 30 min at 20,000×g, and the recombinant proteins were affinity purified on glutathione-Sepharose and eluted in Buffer A containing 20 mM glutathione and 0.27 M sucrose.

Expression and purification of AMPK-related kinases In 293 cells. Twenty 10 cm diameter dishes of 293 cells were cultured and each dish transfected with 5 μg of the pEBG-2T construct encoding wild type AMPK-related kinase using a modified calcium phosphate method (Alessi et al., 1996). 36 h post-transfection, the cells were lysed in 1 ml of ice-cold Lysis Buffer, the lysates pooled and centrifuged at 4° C. for 10 min at 13,000×g. The GST-fusion proteins were purified by affinity chromatography on glutathione-Sepharose and eluted in Buffer A containing 20 mM glutathione and 0.27 M sucrose.

Expression and purification of LKB1:STRAD:MO25 complex in 293 cells. Different combinations of GST-tagged LKB1, FLAG-tagged STRADα or STRADβ, and Myc-tagged MO25α or MO25β were expressed in 293 cells and the complexes purified on glutathione-Sepharose as described previously (Example 1; Boudeau et al., 2003).

Measurement of activation of AMPK-related kinases AMPKα1 catalytic subunit and AMPK-related kinases was measured following their phosphorylation with LKB1 as follows. 1-2 μg of AMPKα1 catalytic domain or AMPK-related kinase, were incubated with or without 0.1-1 μg of wild type of the indicated LKB1 complex in Buffer A containing 5 mM MgAcetate and 0.1 mM ATP, in a final volume of 20 μl. After incubation at 30° C. for the times indicated in the figure legend, AMPKα1 catalytic domain or AMPK-related kinase activities were determined by adding 30 μl of 5 mM magnesium acetate, 0.1 mM [γ-³²P]-ATP (300 cpm/pmol) and 200 μM AMARA peptide (AMARAASAAALARRR (Dale et al., 1995)) as substrate. After incubation for 5-20 min at 30° C., incorporation of ³²P phosphate into the peptide substrate was determined by applying the reaction mixture onto P81 phosphocellulose paper and scintillation counting after washing the papers in phosphoric acid as described previously (Alessi et al., 1995). One Unit (U) of activity was defined as that which catalysed the incorporation of 1 nmol of ³²P into the substrate. Time course reactions for the second stage of the assay were performed in order to ensure that the rate of phosphorylation was occurring linearly with time. Kinetic data was analysed according to the Michaelis-Menten relationship by non-linear regression using the computer program GraphPad Prism (GraphPad Software Inc, San Diego, USA).

Mapping the sites on BRSK2, NUAK2 and MARK3 phosphorylated by the LKB1 complex. The wild type and mutant AMPK-related kinases (5 μg) were incubated for 30 min with 1-2 μg of wild type LKB1:STRADα:MO25β in Buffer A containing 5 mM magnesium acetate and 100 μM [γ-³²P]-ATP (5000 cpm/pmol) in a total reaction volume of 30 μl. After 30 min, the reactions were terminated by adding SDS to a final concentration of 1% (w/v) and dithiothreitol to 10 mM and heated at 100° C. for 1 min. After cooling, 4-vinylpyridine was added to a concentration of 50 mM, and the samples were left on a shaking platform for 30 min at room temperature to alkylate cysteine residues and then subjected to electrophoresis on a BisTris 4-12% polyacrylamide gel. The gels were stained with Coomasie R250, autoradiographed and the bands corresponding to phosphorylated AMPK-related kinases excised and cut into smaller pieces. There were washed sequentially for 15 minutes on a vibrating platform with 1 ml of the following: water, a 1:1 mixture of water and acetonitrile, 0.1 M ammonium bicarbonate, a 1:1 mixture of 0.2 M ammonium bicarbonate and acetonitrile and finally acetonitrile. The gel pieces were dried by rotary evaporation and incubated in 0.1 ml of 50 mM ammonium bicarbonate, 0.1% (by mass) n-octyl-glucoside containing 1 μg of alkylated trypsin. After 16 h, 0.1 ml of acetonitrile was added and the mixture incubated on a shaking platform for 10 min. The supernatant was removed and the gel pieces were further washed for 10 min in 0.3 ml of 50 mM ammonium bicarbonate, and 0.1% (v/v) trifluoroacetic acid. The combined supernatants, containing>90% of the ³²P-radioactivity, were chromatographed on a Vydac 218TP5215 C₁₈ column (Separations Group, Hesperia, Calif.) equilibrated in 0.1% (by vol) trifluoroacetic acid in water. The column was developed with a linear acetonitrile gradient (diagonal line) at a flow rate of 0.2 ml/min and fractions of 0.1 ml were collected.

Phosphopeptide sequence analysis. Isolated phosphopeptides were analysed on an Applied Biosystems 4700 Proteomics Analyser (MALDI-TOF-TOF) using 5 mg/ml alpha cyannocinnamic acid as the matrix. Spectra were acquired in both reflectron and linear modes and the sequence of potential phosphopeptides were confirmed by performing MALDI-MS/MS on selected masses. The characteristic loss of phosphoric acid (M-98 Da) from the parent phosphopeptide as well as the neutral loss of dehydroalanine (−69) for phosphoserine or dehydroaminobutyric acid (−83) for phosphothreonine were used to assign the position of the phosphorylation site(s). The site of phosphorylation of all the ³²P-labelled peptides was determined by solid-phase Edman degradation on an Applied Biosystems 494A sequenator of the peptide coupled to Sequelon-AA membrane (Milligen) as described previously (Campbell and Morrice, 2002).

Identification of T-loop Phosphorylation Site In MELK. The tryptic digest of GST-MELK that had been expressed and purified from E. coli was acidified with 0.25M acetic acid in 30% acetonitrile (v/v) and 2 μl (settled volume) of PHOS-select Iron chelate gel (Sigma) was added. The sample was shaken for 30 min and the resin collected in a microC18 ZipTip (Millipore), washed with 2×25 μl 0.25 M acetic acid in 30% acetonitrile (v/v) and eluted with 25 μl 0.4 M NH₄OH. The eluate was analysed by MALDI-TOF-TOF mass spectrometry and T-loop phosphopeptide was identified and sequenced by ms/ms.

Immunoprecipitation and Assay of Endogenous AMPK and AMPK-Related Kinase

0.1-1 mg of Hela or MEF lysate protein was incubated at 4° C. for 1 h on a shaking platform with 5 μg of the corresponding antibody, which had been previously conjugated to 5 μl of protein G-Sepharose. The immunoprecipitates were washed twice with 1 ml of Lysis Buffer containing 0.5 M NaCl, and twice with 1 ml of Buffer A. Phosphotransferase activity towards the AMARA peptide was then measured in a total assay volume of 50 μl as described above.

Immunoprecipitation and Assay of Endogenous LKB1 Employing LKBtide Substrate.

0.1-1 mg Hela or MEF cell lysate protein was incubated at 4° C. for 1 h on a shaking platform with 5 μl of protein G-Sepharose conjugated to 5 μg of human LKB1 antibody. The immunoprecipitates were washed twice with 1 ml of Lysis Buffer containing 0.5 M NaCl, and twice with 1 ml of Buffer A. Phosphotransferase activity towards the LKBtide peptide (LSNLYHQGKFLQTFCGSPLYRRR residues 241-260 of human NUAK2 with 3 additional Arg residues added to the C-terminal to enable binding to P81 paper), was then measured in a total assay volume of 50 μl consisting of 50 mM Tris/HCl pH 7.5, 0.1 mM EGTA, 0.1% (by vol) 2-mercaptoethanol, 10 mM magnesium acetate, 0.1 mM [γ³²P]ATP (˜200 cpm/pmol) and 200 μM LKB1tide peptide. The assays were carried out at 30° C. with continuous shaking, to keep the immunoprecipitates in suspension, and were terminated after 10 min by applying 40 μl of the reaction mixture onto p81 membranes. The p81 membranes were washed in phosphoric acid, and the incorporated radioactivity was measured by scintillation counting as described previously for MAP kinase (Alessi et al., 1995).

### REFERENCES FOR MATERIALS & METHODS SECTION

Alessi, D. R., Andjelkovic, M., Caudwell, B., Cron, P., Morrice, N., Cohen, P. and Hemmings, B. A. (1996) Mechanism of activation of protein kinase B by insulin and IGF-1. EMBO J, 15, 6541-6551.
Alessi, D. R., Cohen, P., Ashworth, A., Cowley, S., Leevers, S. J. and Marshall, C. J. (1995) Assay and expression of mitogen-activated protein kinase, MAP kinase kinase, and Raf. Methods Enzymol, 255, 279-290.
Boudeau, J., Baas, A. F., Deak, M., Morrice, N. A., Kieloch, A., Schutkowski, M., Prescott, A. R., Clevers, H. C. and Alessi, D. R. (2003) MO25alpha/beta interact with STRADalpha/beta enhancing their ability to bind, activate and localize LKB1 in the cytoplasm. EMBO J, 22, 5102-5114.
Bradford, M. M. (1976) A rapid and sensitive method for the quantitation of microgram quantities of protein utilizing the principle of protein-dye binding. Anal Biochem, 72, 248-254.
Campbell, D. G. and Morrice, N. A. (2002) Identification of Protein Phosphorylation Sites by a Combination of Mass Spectrometry and Solid Phase Edman Sequencing. J. Biomol. Techn., 13, 121-132.
Dale, S., Wilson, W. A., Edelman, A. M. and Hardie, D. G. (1995) Similar substrate recognition motifs for mammalian AMP-activated protein kinase, higher plant HMG-CoA reductase kinase-A, yeast SNF1, and mammalian calmodulin-dependent protein kinase I. FEBS Lett, 361, 191-195.
Hawley, S. A., Boudeau, J., Reid, J. L., Mustard, K. J., Udd, L., Makela, T. P., Alessi, D. R. and Hardie, D. G. (2003) Complexes between the LKB1 tumor suppressor, STRADalpha/beta and MO25alpha/beta are upstream kinases in the AMP-activated protein kinase cascade. J Biol, 2, 28.
Sapkota, G. P., Deak, M., Kieloch, A., Morrice, N., Goodarzi, A. A., Smythe, C., Shiloh, Y., Lees-Miller, S. P. and Alessi, D. R. (2002) Ionizing radiation induces ataxia telangiectasia mutated kinase (ATM)-mediated phosphorylation of LKB1/STK11 at Thr-366. Biochem J, 368, 507-516.
Sapkota, G. P., Kieloch, A., Lizcano, J. M., Lain, S., Arthur, J. S., Williams, M. R., Morrice, N., Deak, M. and Alessi, D. R. (2001) Phosphorylation of the protein kinase mutated in Peutz-Jeghers Cancer Syndrome, LKB1/STK11, at Ser431 by p90RSK and cAMP-dependent protein kinase, but not its farnesylation at Cys433, is essential for LKB1 to suppress cell growth. J Biol Chem, 276, 19469-19482.
Scott, J. W., Norman, D. G., Hawley, S. A., Kontogiannis, L. and Hardie, D. G. (2002) Protein kinase substrate recognition studied using the recombinant catalytic domain of AMP-activated protein kinase and a model substrate. J Mol Biol, 317, 309-323.
Sugden, C., Crawford, R. M., Halford, N. G. and Hardie, D. G. (1999) Regulation of spinach SNF1-related (SnRK1) kinases by protein kinases and phosphatases is associated with phosphorylation of the T loop and is regulated by 5′-AMP. Plant J, 19, 433-439.

### REFERENCES

Alessi, D. R. (2001) Discovery of PDK1, one of the missing links in insulin signal transduction. Biochem Soc Trans, 29, 1-14.
Baas, A. F., Boudeau, J., Sapkota, G. P., Smit, L., Medema, R., Morrice, N. A., Alessi, D. R. and Clevers, H. C. (2003) Activation of the tumour suppressor kinase LKB1 by the STE20-like pseudokinase STRAD. EMBO J, 22, 3062-3072.
Bardeesy, N., Sinha, M., Hezel, A. F., Signoretti, S., Hathaway, N. A., Sharpless, N. E., Loda, M., Carrasco, D. R. and DePinho, R. A. (2002) Loss of the Lkb1 tumour suppressor provokes intestinal polyposis but resistance to transformation. Nature, 419, 162-167.
Boudeau, J., Baas, A. F., Deak, M., Morrice, N. A., Kieloch, A., Schutkowski, M., Prescott, A. R., Clevers, H. C. and Alessi, D. R. (2003a) MO25alpha/beta interact with STRADalpha/beta enhancing their ability to bind, activate and localize LKB1 in the cytoplasm. EMBO J, 22, 5102-5114.
Boudeau, J., Deak, M., Lawlor, M. A., Morrice, N. A. and Alessi, D. R. (2003b) Heat-shock protein 90 and Cdc37 interact with LKB1 and regulate its stability. Biochem J, 370, 849-857.
Buchet-Poyau, K., Mehenni, H., Radhakrishna, U. and Antonarakis, S. E. (2002) Search for the second Peutz-Jeghers syndrome locus: exclusion of the STK13, PRKCG, KLK10, and PSCD2 genes on chromosome 19 and the STK11IP gene on chromosome 2. Cytogenet Genome Res, 97, 171-178.
Corton, J. M., Gillespie, J. G., Hawley, S. A. and Hardie, D. G. (1995) 5-aminoimidazole-4-carboxamide ribonucleoside. A specific method for activating AMP-activated protein kinase in intact cells? Eur J Biochem, 229, 558-565.
Dale, S., Wilson, W. A., Edelman, A. M. and Hardie, D. G. (1995) Similar substrate recognition motifs for mammalian AMP-activated protein kinase, higher plant HMG-CoA reductase kinase-A, yeast SNF1, and mammalian calmodulin-dependent protein kinase I. FEBS Lett, 361, 191-195.
Drewes, G., Ebneth, A., Preuss, U., Mandelkow, E. M. and Mandelkow, E. (1997) MARK, a novel family of protein kinases that phosphorylate microtubule-associated proteins and trigger microtubule disruption. Cell, 89, 297-308.
Drewes, G. and Nurse, P. (2003) The protein kinase kin1, the fission yeast orthologue of mammalian MARK/PAR-1, localises to new cell ends after mitosis and is important for bipolar growth. FEBS Lett, 554, 45-49.
Feldman, J. D., Vician, L., Crispino, M., Hoe, W., Baudry, M. and Herschman, H. R. (2000) The salt-inducible kinase, SIK, is induced by depolarization in brain. J Neurochem, 74, 2227-2238.
Guo, S. and Kemphues, K. J. (1995) par-1, a gene required for establishing polarity in C. elegans embryos, encodes a putative Ser/Thr kinase that is asymmetrically distributed. Cell, 81, 611-620.
Hardie, D. G., Scott, J. W., Pan, D. A. and Hudson, E. R. (2003) Management of cellular energy by the AMP-activated protein kinase system. FEBS Lett, 546, 113-120.
Hawley, S. A., Boudeau, J., Reid, J. L., Mustard, K. J., Udd, L., Makela, T. P., Alessi, D. R. and Hardie, D. G. (2003) Complexes between the LKB1 tumor suppressor, STRADalpha/beta and MO25alpha/beta are upstream kinases in the AMP-activated protein kinase cascade. J Biol, 2, 28.
Hawley, S. A., Gadalla, A. E., Olsen, G. S. and Hardie, D. G. (2002) The antidiabetic drug metformin activates the AMP-activated protein kinase cascade via an adenine nucleotide-independent mechanism. Diabetes, 51, 2420-2425.
Hemminki, A., Markie, D., Tomlinson, I., Avizienyle, E., Roth, S., Loukola, A., Bignell, G., Warren, W., Aminoff, M., Hoglund, P., Jarvinen, H., Kristo, P., Pelin, K., Ridanpaa, M., Salovaara, R., Toro, T., Bodmer, W., Olschwang, S., Olsen, A. S., Stratton, M. R., de la Chapelle, A. and Aaltonen, L. A. (1998) A serine/threonine kinase gene defective in Peutz-Jeghers syndrome. Nature, 391, 184-187.
Heyer, B. S., Kochanowski, H. and Solter, D. (1999) Expression of Melk, a new protein kinase, during early mouse development. Dev Dyn, 215, 344-351.
Heyer, B. S., Warsowe, J., Solter, D., Knowles, B. B. and Ackerman, S. L. (1997) New member of the Snf1/AMPK kinase family, Melk, is expressed in the mouse egg and preimplantation embryo. Mol Reprod Dev, 47, 148-156.
Hong, S. P., Leiper, F. C., Woods, A., Carling, D. and Carlson, M. (2003) Activation of yeast Snf1 and mammalian AMP-activated protein kinase by upstream kinases. Proc Natl Acad Sci USA, 100, 8839-8843.
Horike, N., Takemori, H., Katoh, Y., Doi, J., Min, L., Asano, T., Sun, X. J., Yamamoto, H., Kasayama, S., Muraoka, M., Nonaka, Y. and Okamoto, M. (2003) Adipose-specific expression, phosphorylation of Ser794 in insulin receptor substrate-1, and activation in diabetic animals of salt-inducible kinase-2. J Biol Chem, 278, 18440-18447.
Jakobsen, S. N., Hardie, D. G., Morrice, N. and Tornqvist, H. E. (2001) 5′-AMP-activated protein kinase phosphorylates IRS-1 on Ser-789 in mouse C2C12 myotubes in response to 5-aminoimidazole-4-carboxamide riboside. J Biol Chem, 276, 46912-46916.
Jenne, D. E., Reimann, H., Nezu, J., Friedel, W., Loff, S., Jeschke, R., Muller, O., Back, W. and Zimmer, M. (1998) Peutz-Jeghers syndrome is caused by mutations in a novel serine threonine kinase. Nat Genet, 18, 38-43.
Jishage, K. I., Nezu, J. I., Kawase, Y., Iwata, T., Watanabe, M., Miyoshi, A., Ose, A., Habu, K., Kake, T., Kamada, N., Ueda, O., Kinoshita, M., Jenne, D. E., Shimane, M. and Suzuki, H. (2002) Role of Lkb1, the causative gene of Peutz-Jegher's syndrome, in embryogenesis and polyposis. Proc Natl Acad Sci USA, 99, 8903-8908.
Johnson, L. N., Noble, M. E. and Owen, D. J. (1996) Active and inactive protein kinases: structural basis for regulation. Cell, 85, 149-158.
Kemp, B. E., Mitchelhill, K. I., Stapleton, D., Michell, B. J., Chen, Z. P. and Witters, L. A. (1999) Dealing with energy demand: the AMP-activated protein kinase. Trends Biochem Sci, 24, 22-25.
Kemphues, K. J., Priess, J. R., Morton, D. G. and Cheng, N. S. (1988) Identification of genes required for cytoplasmic localization in early C. elegans embryos. Cell, 52, 311-320.
Lefebvre, D. L., Bai, Y., Shahmolky, N., Sharma, M., Poon, R., Drucker, D. J. and Rosen, C. F. (2001) Identification and characterization of a novel sucrose-non-fermenting protein kinase/AMP-activated protein kinase-related protein kinase, SNARK. Biochem J, 355, 297-305.
Manning, G., Whyte, D. B., Martinez, R., Hunter, T. and Sudarsanam, S. (2002) The protein kinase complement of the human genome. Science, 298, 1912-1934.
Martin, S. G. and St Johnston, D. (2003) A role for Drosophila LKB1 in anterior-posterior axis formation and epithelial polarity. Nature, 421, 379-384.
Milburn, C. C., Boudeau, J., Deak, M., Alessi, D. R. and van aalten, D. M. F. (2003) Crystal structure of MO25α in complex with the C-terminus of the pseudokinase STE20-Related ADaptor (STRAD). Nature Struct. Biol., In the press.
Miyoshi, H., Nakau, M., Ishikawa, T. O., Seldin, M. F., Oshima, M. and Taketo, M. M. (2002) Gastrointestinal hamartomatous polyposis in Lkb1 heterozygous knockout mice. Cancer Res, 62, 2261-2266.
Morton, D. G., Roos, J. M. and Kemphues, K. J. (1992) par-4, a gene required for cytoplasmic localization and determination of specific cell types in Caenorbabditis elegans embryogenesis. Genetics, 130, 771-790.
Nath, N., McCartney, R. R. and Schmidt, M. C. (2003) Yeast Pak1 kinase associates with and activates Snf1 . Mol Cell Biol, 23, 3909-3917.
Ossipova, O., Bardeesy, N., DePinho, R. A. and Green, J. B. (2003) LKB1 (XEEK1) regulates Wnt signalling in vertebrate development. Nat Cell Biol, 5, 889-894.
Peng, C. Y., Graves, P. R., Ogg, S., Thoma, R. S., Byrnes, M. J., 3rd, Wu, Z., Stephenson, M. T. and Piwnica-Worms, H. (1998) C-TAK1 protein kinase phosphorylates human Cdc25C on serine 216 and promotes 14-3-3 protein binding. Cell Growth Differ, 9, 197-208.
Resta, N., Stella, A., Susca, F. C., Di Giacomo, M., Forleo, G., Miccolis, I., Rossini, F. P., Genuardi, M., Piepoli, A., Grammatico, P. and Guanti,

1. A method for identifying a compound which modulates the activation or phosphorylation of an AMP-activated protein kinase or an AMP-activated protein kinase subfamily member in a cell, the method comprising the steps of (1) determining whether a test compound modulates the protein kinase activity of LKB1 and (2) selecting a compound which modulates the protein kinase activity of LKB1, wherein the LKB1 is in a preparation comprising STRAD or MO25 or both.

2. The method of claim 1 wherein the LKB1, STRAD or MO25 is recombinant and is expressed from a recombinant nucleic acid.

3. A purified preparation comprising LKB1, STRAD and recombinant MO25 expressed from a recombinant nucleic acid.

4. The preparation of claim 3 comprising recombinant LKB1 expressed from a recombinant nucleic acid.

5. The preparation of claim 4 comprising recombinant STRAD expressed from a recombinant nucleic acid.

6. A cell capable of expressing LKB1, STRAD and overexpressed or recombinant MO25 expressed from a recombinant nucleic acid.

7. The cell of claim 6 comprising a recombinant nucleic acid encoding MO25.

8. The cell of claim 7 comprising a recombinant nucleic acid encoding LKB1.

9. The cell of claim 8 comprising a recombinant nucleic acid encoding STRAD.

10. A cell comprising LKB1, STRAD and overexpressed or recombinant MO25 expressed from a recombinant nucleic acid.

11. A cell according to claim 10 comprising recombinant LKB1 expressed from a recombinant nucleic acid.

12. A cell according to claim 10 comprising recombinant STRAD expressed from a recombinant nucleic acid.

13. (canceled)

14. A method for making a purified preparation comprising LKB1, STRAD and recombinant MO25 expressed from a recombinant nucleic acid comprising:
selecting a cell according to claim 10 and purifying the preparation from the cell.

15. (canceled)

16. The preparation of claim 3 wherein the LKB1:STRAD:MO25 ratios are 1:1:1.

17. (canceled)

18. (canceled)

19. A method for identifying a compound for modulating cellular LKB1 activity, the method comprising the steps of (1) determining whether a test compound modulates the LKB1 protein kinase activity of a preparation according to claim 3 and (2) selecting a compound which modulates said LKB1 protein kinase activity.

20. The method of claim 19 wherein the LKB1 protein kinase activity is measured using an AMPK or an AMPK subfamily member or a fragment thereof as the substrate.

21. A kit of parts comprising the preparation of claim 3.

22. A kit of parts according to claim 21 further comprising (1) an AMPK or an AMPK subfamily member, or recombinant polynucleotide encoding AMPK or AMPK subfamily member or a fragment thereof.

23. A method for overexpressing LKB1 comprising the steps of (1) selecting a cell according to claim 6 in which to overexpress LKB1 and (2) overexpressing LKB1 in the selected cell.

24. A method according to claim 23 further comprising preparing LKB1 from the cell.

25. A method for identifying a putative binding partner for MO25 comprising the steps of (1) providing an amino acid sequence of at least the C-terminal three amino acids of a test putative binding partner (2) selecting a putative binding partner having the C-terminal amino acid sequence Trp-Glu/Asp-Phe.

26. The method of claim 25 further comprising the step of determining that the selected putative binding partner binds to MO25.

27. A method for identifying a genetic difference associated with PJS (Peutz-Jeghers Syndrome) comprising the steps of (1) investigating the sequence of a gene encoding a MO25 isoform in at least one patient having PJS (2) identifying any difference between the said patient sequence and equivalent sequence from an individual without PJS.

28. A method for determining whether an individual is susceptible to PJS comprising the steps of determining whether the test individual has a genetic difference identified as associated with PJS by a method according to claim 27.

29. A method for identifying a compound which activates an AMPK or an AMPK subfamily member by a similar mechanism to metformin or phenformin or AICA riboside comprising comparing the effect of a test compound on the activation of the AMPK or the AMPK subfamily member by a preparation according to claim 3 with the effect of metformin or phenformin or AICA riboside on the activation of the AMPK or the AMPK subfamily member and selecting the compound with a similar effect.

30. (canceled)

31. The kit of parts of claim 22 wherein the AMPK subfamily member is or comprises an AMPKα1 or AMPKα2 polypeptide.

32. The kit of parts of claim 22 wherein the AMPK subfamily member is or comprises a NUAK1, NUAK2, BRSK1, BRSK2, SIK, QIK, QSK, MARK1, MARK2, MARK3, MARK4 or MELK polypeptide.

33. A peptide substrate for LKB1 comprising the amino acid sequence SEQ ID NO:23, SEQ ID NO:24, SEQ ID NO:25, SEQ ID NO:29, SEQ ID NO:31, SEQ ID NO:33, SEQ ID NO:35, SEQ ID NO:37, LSNLYHQGKFLQTFCGSPLY (SEQ ID NO:16), FGNFYKSGEPLSTWCGSPPY (SEQ ID NO:17), LSNMMSDGEFLRTSCGSPNY (SEQ ID NO:18), MASLQVGDSLLETSCGSPHY (SEQ ID NO:19), FSNEFTVGGKLDTFCGSPPY (SEQ ID NO:20), or AKPKGNKDYHLQTCCGSLAY (SEQ ID NO:21); or said amino acid sequence with from one to four substitutions therein at any position other than the underlined residue and/or a conservative substitution at the underlined residue; or at least ten contiguous residues of said sequence encompassing the underlined residue.

34. A peptide substrate for LKB1 according to claim 1 consisting of the amino acid sequence LSNLYHQGKFLQTFCGSPLY (SEQ ID NO:16), LSNLYHQGKFLQTFCGSPLYRRR (SEQ ID NO:23), SNLYHQGKFLQTFCGSPLY SEQ ID NO:24), SNLYHQGKFLQTFCGSPLYRRR (SEQ ID NO:25), FGNFYKSGEPLSTWCGSPPY (SEQ ID NO:17), FGNFYKSGEPLSTWCGSPPYRRR (SEQ ID NO:29), LSNMMSDGEFLRTSCGSPNY (SEQ ID NO:18), LSNMMSDGEFLRTSCGSPNYRRR (SEQ ID NO:31), MASLQVGDSLLETSCGSPHY (SEQ ID NO: 19), MASLQVGDSLLETSCGSPHYRRR (SEQ ID NO:33), or FSNEFTVGGKLDTFCGSPPY (SEQ ID NO: 20), FSNEFTVGGKLDTFCGSPPYRRR (SEQ ID NO: 35), AKPKGNKDYHLQTCCGSLAY (SEQ ID NO: 21), or AKPKGNKDYHLQTCCGSLAYRRR (SEQ ID NO: 37).

35. An antibody reactive with a peptide antigen having the amino acid sequence MVAGLTLGKGPESPDGDVS (SEQ ID NO: 38) (residues 1-20 of human BRSK1), LSWGAGLKGQKVATSYESSL (SEQ ID NO: 39) (residues 655-674 of human BRSK2), MEGAAAPVAGDRPDLGLGAPG (SEQ ID NO: 40) (residues 1-21 of human NUAK1), TDCQEVTATYRQALRVCSKLT (SEQ ID NO: 41) (residues 653-673 of human NUAK2), MVMADGPRHLQRGPVRVGFYD (SEQ ID NO: 42) (residues 1-21 of human QIK), MVIMSEFSADPAGQGQGQQK (SEQ ID NO: 43) (residues 1-20 of human SIK), GDCEMEDLMPCSLGTFVLVQ (SEQ ID NO: 44) (residues 765-784 of human SIK), TDILLSYKHPEVSFSMEQAGV (SEQ ID NO: 45) (residues 1349-1369 of human QSK), SGTSIAFKNIASKIANELKL (SEQ ID NO: 46) (residues 776-795 of human MARK1), MSSRTVLAPGNDRNSDTHGT (SEQ ID NO: 47) (residues 1-20 of human MARK4), MKDYDELLKYYELHETIGTG (SEQ ID NO: 48) (residues 1-20 of human MELK), CTSPPDSFLDDHHLTR (SEQ ID NO: 49) (residues 344-358 of rat AMPKα1), CDPMKRATIKDIRE (SEQ ID NO: 50) (residues 252 to 264 of rat AMPKα1).


SEQUENCE LISTING (158 sequences)

SEQ ID NO: 1 - protein, 550 aa, Homo sapiens
MATAEKQKHDGRVKIGHYILGDTLGVGTFGKVKVGKHELTGHKVAVKILNRQKIRSLDVVGKIRREIQNLKLFRHPHIIKLYQVISTPSDIFMVMEYVSGGELFDYICKNGRLDEKESRRLFQQILSGVDYCHRHMVVHRDLKPENVLLDAHMNAKIADFGLSNMMSDGEFLRTSCGSPNYAAPEVISGRLYAGPEVDIWSSGVILYALLCGTLPFDDDHVPTLFKKICDGIFYTPQYLNPSVISLLKHMLQVDPMKRATIKDIREHEWFKQDLPKYLFPEDPSYSSTMIDDEALKEVCEKFECSEEEVLSCLYNRNHQDPLAVAYHLIIDNRRIMNEAKDFYLATSPPDSFLDDHHLTRPHPERVPFLVAETPRARHTLDELNPQKSKHQGVRKAKWHLGIRSQSRPNDIMAEVCRAIKQLDYEWKVVNPYYLRVRRKNPVTSTYSKMSLQLYQVDSRTYLLDFRSIDDEITEAKSGTATPQRSGSVSNYRSCQRSDSDAEAQGKSSEVSLTSSVTSLDSSPVDLTPRPGSHTIEFFEMCANLIKILAQ

SEQ ID NO: 2 - protein, 550 aa, Homo sapiens
MATAEKQKHDGRVKIGHYILGDTLGVGTFGKVKVGKHELTGHKVAVKILNRQKIRSLDVVGKIRREIQNLKLFRHPHIIKLYQVISTPSDIFMVMEYVSGGELFDYICKNGRLDEKESRRLFQQILSGVDYCHRHMVVHRDLKPENVLLDAHMNAKIADFGLSNMMSDGEFLRTSCGSPNYAAPEVISGRLYAGPEVDIWSSGVILYALLCGTLPFDDDHVPTLFKKICDGIFYTPQYLNPSVISLLKHMLQVDPMKRASIKDIREHEWFKQDLPKYLFPEDPSYSSTMIDDEALKEVCEKFECSEEEVLSCLYNRNHQDPLAVAYHLIIDNRRIMNEAKDFYLATSPPDSFLDDHHLTRPHPERVPFLVAETPRARHTLDELNPQKSKHQGVRKAKWHLGIRSQSRPNDIMAEVCRAIKQLDYEWKVVNPYYLRVRRKNPVTSTYSKMSLQLYQVDSRTYLLDFRSIDDEITEAKSGTATPQRSGSVSNYRSCQRSDSDAEAQGKSSEVSLTSSVTSLDSSPVDLTPRPGSHTIEFFEMCANLIKILAQ

SEQ ID NO: 3 - protein, 550 aa, Homo sapiens; identical to SEQ ID NO: 2

SEQ ID NO: 4 - protein, 520 aa, Homo sapiens
GEHQLTGHKVAVKILNRQKIRSLDVVGKIKREIQNLKLFRHPHIIKLYQVISTPTDFFMVMEYVSGGELFDYICKHGRVEEMEARRLFQQILSAVDYCHRHMVVHRDLKPENVLLDAHMNAKIADFGLSNMMSDGEFLRTSCGSPNYAAPEVISGRLYAGPEVDIWSCGVILYALLCGTLPFDDEHVPTLFKKIRGGVFYIPEYLNRSVATLLMHMLQVDPLKRATIKDIREHEWFKQDLPSYLFPEDPSYDANVIDDEAVKEVCEKFECTESEVMNSLYSGDPQDQLAVAYHLIIDNRRIMNQASEFYLASSPPSGSFMDDSAMHIPPGLKPHPERMPPLIADSPKARCPLDALNTTKPKSLAVKKAKWHLGIRSQSKPYDIMAEVYRAMKQLDFEWKVVNAYHLRVRRKNPVTGNYVKMSLQLYLVDNRSYLLDFKSIDDEVVEQRSGSSTPQRSCSAAGLHRPRSSFDSTTAESHSLSGSLTGSLTGSTLSSVSPRLGSHTMDFFEMCASLITTLAR

SEQ ID NO: 5 - protein, 552 aa, Homo sapiens
MAEKQKHDGRVKIGHYVLGDTLGVGTFGKVKIGEHQLTGHKVAVKILNRQKIRSLDVVGKIKREIQNLKLFRHPHIIKLYQVISTPTDFFMVMEYVSGGELFDYICKHGRVEEMEARRLFQQILSAVDYCHRHMVVHRDLKPENVLLDAHMNAKIADFGLSNMMSDGEFLRTSCGSPNYAAPEVISGRLYAGPEVDIWSCGVILYALLCGTLPFDDEHVPTLFKKIRGGVFYIPEYLNRSVATLLMHMLQVDPLKRATIKDIREHEWFKQDLPSYLFPEDPSYDANVIDDEAVKEVCEKFECTESEVMNSLYSGDPQDQLAVAYHLIIDNRRIMNQASEFYLASSPPSGSFMDDSAMHIPPGLKPHPERMPPLIADSPKARCPLDALNTTKPKSLAVKKAKWHLGIRSQSKPYDIMAEVYRAMKQLDFEWKVVNAYHLRVRRKNPVTGNYVKMSLQLYLVDNRSYLLDFKSIDDEVVEQRSGSSTPQRSCSAAGLHRPRSSFDSTTAESHSLSGSLTGSLTGSTLSSVSPRLGSHTMDFFEMCASLITTLAR

SEQ ID NO: 6 - protein, 433 aa, Homo sapiens
MEVVDPQQLGMFTEGELMSVGMDTFIHRIDSTEVIYQPRRKRAKLIGKYLMGDLLGEGSYGKVKEVLDSETLCRRAVKILKKKKLRRIPNGEANVKKEIQLLRRLRHKNVIQLVDVLYNEEKQKMYMVMEYCVCGMQEMLDSVPEKRFPVCQAHGYFCQLIDGLEYLHSQGIVHKDIKPGNLLLTTGGTLKISDLGVAEALHPFAADDTCRTSQGSPAFQPPEIANGLDTFSGFKVDIWSAGVTLYNITTGLYPFEGDNIYKLFENIGKGSYAIPGDCGPPLSDLLKGMLEYEPAKRFSIRQIRQHSWFRKKHPPAEAPVPIPPSPDTKDRWRSMTVVPYLEDLHGADEDEDLFDIEDDIIYTQDFTVPGQVPEEEASHNGQRRGLPKAVCMNGTEAAQLSTKSRAEGRAPNPARKACSASSKIRRLSACKQQ

SEQ ID NO: 7 - protein, 433 aa, Homo sapiens; identical to SEQ ID NO: 6

SEQ ID NO: 8 - protein, 433 aa, Homo sapiens; identical to SEQ ID NO: 6

SEQ ID NO: 9 - protein, 431 aa, Homo sapiens [sequence omitted]

SEQ ID NO: 10 - protein, 418 aa, Homo sapiens
MSLLDCFCTSRTQVESLRPEKQSETSIHQYLVDEPTLSWSRPSTRASEVLCSTNVSHYELQVEIGRGFDNLTSVHLARHTPTGTLVTIKITNLENCNEERLKALQKAVILSHFFRHPNITTYWTVFTVGSWLWVISPFMAYGSASQLLRTYFPEGMSETLIRNILFGAVRGLNYLHQNGCIHRSIKASHILISGDGLVTLSGLSHLHSLVKHGQRHRAVYDFPQFSTSVQPWLSPELLRQDLHGYNVKSDIYSVGITACELASGQVPFQDMHRTQMLLQKLKGPPYSPLDISIFPQSESRMKNSQSGVDSGIGESVLVSSGTHTVNSDRLHTPSSKTFSPAFFSLVQLCLQQDPEKRPSASSLLSHVFFKQMKEESQDSILSLLPPAYNKPSISLPPVLPWTEPECDFPDEKDSYWEF

SEQ ID NO: 11 - protein, 341 aa, Homo sapiens
MPFPFGKSHKSPADIVKNLKESMAVLEKQDISDKKAEKATEEVSKNLVAMKEILYGTNEKEPQTEAVAQLAQELYNSGLLSTLVADLQLIDFEGKKDVAQIFNNILRRQIGTRTPTVEYICTQQNILFMLLKGYESPEIALNCGIMLRECIRHEPLAKIILWSEQFYDFFRYVEMSTFDIASDAFATFKDLLTRHKLLSAEFLEQHYDRFFSEYEKLLHSENYVTKRQSLKLLGELLLDRHNFTIMTKYISKPENLKLMMNLLRDKSRNIQFEAFHVFKVFVANPNKTQPILDILLKNQAKLIEFLSKFQNDRTEDEQFNDEKTYLVKQIRDLKRPAQQEA

SEQ ID NO: 12 - protein, 337 aa, Homo sapiens
MKKMPLFSKSHKNPAEIVKILKDNLAILEKQDKKTDKASEEVSKSLQAMKEILCGTNEKEPPTEAVAQLAQELYSSGLLVTLIADLQLIDFEGKKDVTQIFNNILRRQIGTRSPTVEYISAHPHILFMLLKGYEAPQIALRCGIMLRECIRHEPLAKIILFSNQFRDFFKYVELSTFDIASDAFATFKDLLTRHKVLVADFLEQNYDTIFEDYEKLLQSENYVTKRQSLKLLGELILDRHNFAIMTKYISKPENLKLMMNLLRDKSPNIQFEAFHVFKVFVASPHKTQPIVEILLKNQPKLIEFLSSFQKERTDDEQFADEKNYLIKQIRDLKKTAP

SEQ ID NO: 13 - protein, 338 aa, Caenorhabditis elegans
MLKPLFGKADKTPADVVKNLRDALLVIDRHGTNTSERKVEKAIEETAKMLALAKTFIYGSDANEPNNEQVTQLAQEVYNANVLPMLIKHLHKFEFECKKDVASVFNNLLRRQIGTRSPTVEYLAARPEILITLLLGYEQPDIALTCGSMLREAVRHEHLARIVLYSEYFQRFFVFVQSDVFDIATDAFSTFKDLMTKHKNMCAEYLDNNYDRFFGQYSALTNSENYVTRRQSLKLLGELLLDRHNFSTMNKYITSPENLKTVMELLRDKRRNIQYEAFHVFKIFVANPNKPRPITDILTRNRDKLVEFLTAFHNDRTNDEQFNDEKAYLIKQIQELRV

SEQ ID NO: 14 - protein, 636 aa, Caenorhabditis elegans
MDSTTSLPNNVLLKKARPSKIFAVTSANALNVKTEPVIFVKSDDLNQANTPLTGSKFGTHLACIRTSCLHRTVNASNYSTMSDGGLYTSDEPCSSAQAEFRLAAHWESTFTRTREIHCDTGYSSQSPPETTVFIQKSRFPVAEKPGTPELKSFESKKLVQKKSGNASTPTRKLASEEKKAKNTSMGQTPSKLKSPKALKMVKKENEPAIPPNHFEGKVYGYLVDDMSAIGIQPILDKYNEDPEKFFKRFDSKPWFRRKVMPLLFGKSHKSPADVVKTLREVLTILDKLPPPKLDKDGNIQSDKKYDKALDEVSKNVAMIKSFIYGNDSAEPSSEHVVQVAQLAQEVYNANILPMLIKMLPKFEFECKKDVGQIFNNLLRRQIGTRSPTVEYLGARPEILIQLVQGYSVPDIALTCGLMLRESIRHDHLAKIILYSDVFYTFFLYVQSEVFDISSDAFSTFKELTTRHKAIIAEFLDSNYDTFFAQYQNLLNSKNYVTRRQSLKLLGELLLDRHNFNTMTKYISNPDNLRLMMELLRDKSRNIQYEAFHVFKVFVANPNKPKPISDILNRNREKLVEFLSEFHNDRTDDEQFNDEKAYLIKQIQEMKSSPKEAKKPKSKEDENQEPAGPSEGPSTSQ

SEQ ID NO: 15 - protein, 339 aa, Drosophila melanogaster
MPLFGKSQKSPVELVKSLKEAINALEAGDRKVEKAQEDVSKNLVSIKNMLYGSSDAEPPADYVVAQLSQELYNSNLLLLLIQNLHRIDFEGKKHVALIFNNVLRRQIGTRSPTVEYICTKPEILFTLMAGYEDAHPEIALNSGTMLRECARYEALAKIMLHSDEFFKFFRYVEVSTFDIASDAFSTFKELLTRHKLLCAEFLDANYDKFFSQHYQRLLNSENYVTRRQSLKLLGELLLDRHNFTVMTRYISEPENLKLMMNMLKEKSRNIQFEAFHVFKVFVANPNKPKPILDILLRNQTKLVDFLTNFHTDRSEDEQFNDEKAYLIKQIKELKPLPEA

SEQ ID NO: 16 - protein, 20 aa, Artificial
  LKB1 substrate
LSNLYHQGKFLQTFCGSPLY

SEQ ID NO: 17 - protein, 20 aa, Artificial
  LKB1 substrate
FGNFYKSGEPLSTWCGSPPY

SEQ ID NO: 18 - protein, 20 aa, Artificial
  LKB1 substrate
LSNMMSDGEFLRTSCGSPNY

SEQ ID NO: 19 - protein, 20 aa, Artificial
  LKB1 substrate
MASLQVGDSLLETSCGSPHY

SEQ ID NO: 20 - protein, 20 aa, Artificial
  LKB1 substrate
FSNEFTVGGKLDTFCGSPPY

SEQ ID NO: 21 - protein, 20 aa, Artificial
  LKB1 substrate
AKPKGNKDYHLQTCCGSLAY

SEQ ID NO: 22 - protein, 20 aa, Artificial
  LKB1 substrate
LSNLYHQGKFLQTFCGSPLY

SEQ ID NO: 23 - protein, 23 aa, Artificial
  LKB1 substrate
LSNLYHQGKFLQTFCGSPLYRRR

SEQ ID NO: 24 - protein, 19 aa, Artificial
  LKB1 substrate
SNLYHQGKFLQTFCGSPLY

SEQ ID NO: 25 - protein, 22 aa, Artificial
  LKB1 substrate
SNLYHQGKFLQTFCGSPLYRRR

SEQ ID NO: 26 - protein, 20 aa, Artificial
  LKB1 substrate
LSNLYHQGKFLQTFCGSPLY

SEQ ID NO: 27 - protein, 23 aa, Artificial
  LKB1 substrate
LSNLYHQGKFLQTFCGSPLYRRR

SEQ ID NO: 28 - protein, 20 aa, Artificial
  LKB1 substrate
FGNFYKSGEPLSTWCGSPPY

SEQ ID NO: 29 - protein, 23 aa, Artificial
  LKB1 substrate
FGNFYKSGEPLSTWCGSPPYRRR

SEQ ID NO: 30 - protein, 20 aa, Artificial
  LKB1 substrate
LSNMMSDGEFLRTSCGSPNY

SEQ ID NO: 31 - protein, 23 aa, Artificial
  LKB1 substrate
LSNMMSDGEFLRTSCGSPNYRRR

SEQ ID NO: 32 - protein, 20 aa, Artificial
  LKB1 substrate
MASLQVGDSLLETSCGSPHY

SEQ ID NO: 33 - protein, 23 aa, Artificial
  LKB1 substrate
MASLQVGDSLLETSCGSPHYRRR

SEQ ID NO: 34 - protein, 20 aa, Artificial
  LKB1 substrate
FSNEFTVGGKLDTFCGSPPY

SEQ ID NO: 35 - protein, 23 aa, Artificial
  LKB1 substrate
FSNEFTVGGKLDTFCGSPPYRRR

SEQ ID NO: 36 - protein, 20 aa, Artificial
  LKB1 substrate
AKPKGNKDYHLQTCCGSLAY

SEQ ID NO: 37 - protein, 23 aa, Artificial
  LKB1 substrate
AKPKGNKDYHLQTCCGSLAYRRR

SEQ ID NO: 38 - protein, 19 aa, Homo sapiens
MVAGLTLGKGPESPDGDVS

SEQ ID NO: 39 - protein, 20 aa, Homo sapiens
LSWGAGLKGQKVATSYESSL

SEQ ID NO: 40 - protein, 21 aa, Homo sapiens
MEGAAAPVAGDRPDLGLGAPG

SEQ ID NO: 41 - protein, 21 aa, Homo sapiens
TDCQEVTATYRQALRVCSKLT

SEQ ID NO: 42 - protein, 21 aa, Homo sapiens
MVMADGPRHLQRGPVRVGFYD

SEQ ID NO: 43 - protein, 20 aa, Homo sapiens
MVIMSEFSADPAGQGQGQQK

SEQ ID NO: 44 - protein, 20 aa, Homo sapiens
GDCEMEDLMPCSLGTFVLVQ

SEQ ID NO: 45 - protein, 21 aa, Homo sapiens
TDILLSYKHPEVSFSMEQAGV

SEQ ID NO: 46 - protein, 20 aa, Homo sapiens
SGTSIAFKNIASKIANELKL

SEQ ID NO: 47 - protein, 20 aa, Homo sapiens
MSSRTVLAPGNDRNSDTHGT

SEQ ID NO: 48 - protein, 20 aa, Homo sapiens
MKDYDELLKYYELHETIGTG

SEQ ID NO: 49 - protein, 16 aa, Homo sapiens
CTSPPDSFLDDHHLTR

SEQ ID NO: 50 - protein, 14 aa, Homo sapiens
CDPMKRATIKDIRE

SEQ ID NO: 51 - protein, 12 aa, Artificial
  C-terminal 12 residues STRAD alpha
NLEELEVDDWEF

SEQ ID NO: 52 - protein, 12 aa, Artificial
  C-terminal 12 residues STRAD alpha, last residue mutated to Ala
NLEELEVDDWEA

SEQ ID NO: 53 - protein, 12 aa, Artificial
  C-terminal 12 residues STRAD alpha, third last residue mutated to Ala
NLEELEVDDAEF

SEQ ID NO: 54 - protein, 12 aa, Artificial
  C-terminal 12 residues STRAD alpha, second last residue mutated to Ala
NLEELEVDDWAF

SEQ ID NO: 55 - protein, 6 aa, Artificial
  C-terminal 6 residues STRAD alpha
VDDWEF

SEQ ID NO: 56 - protein, 547 aa, Homo sapiens
MAEPSGSPVHVQLPQQAAPVTAAAAAAPAAATAAPAPAAPAAPAPAPAPAPAAQAVGWPICRDAYELQEVIGSGATAVVQAALCKPRQERVAIKRINLEKCQTSMDELLKEIQAMSQCSHPNVVTYYTSFVVKDELWLVMKLLSGGSMLDIIKYIVNRGEHKNGVLEEAIIATILKEVLEGLDYLHRNGQIHRDLKAGNILLGEDGSVQIADFGVSAFLATGGDVTRNKVRKTFVGTPCWMAPEVMEQVRGYDFKADMWSFGITAIELATGAAPYHKYPPMKVLMLTLQNDPPTLETGVEDKEMMKKYGKSFRKLLSLCLQKDPSKRPTAAELLKCKFFQKAKNREYLIEKLLTRTPDIAQRAKKVRRVPGSSGHLHKTEDGDWEWSDDEMDEKSEEGKAAFSQEKSRRVKEENPEIAVSASTIPEQIQSLSVHDSQGPPNANEDYREASSCAVNLVLRLRNSRKELNDIRFEFTPGRDTADGVSQELFSAGLVDGHDVVIVAANLQKIVDDPKALKTLTFKLASGCDGSEIPDEVKLIGFAQLSVS

SEQ ID NO: 57 - protein, 527 aa, Homo sapiens
MSEDSSALPWSINRDDYELQEVIGSGATAVVQAAYCAPKKEKVAIKRINLEKCQTSMDELLKEIQAMSQCHHPNIVSYYTSFVVKDELWLVMKLLSGGSVLDIIKHIVAKGEHKSGVLDESTIATILREVLEGLEYLHKNGQIHRDVKAGNILLGEDGSVQIADFGVSAFLATGGDITRNKVRKTFVGTPCWMAPEVMEQVRGYDFKADIWSFGITAIELATGAAPYHKYPPMKVLMLTLQNDPPSLETGVQDKEMLKKYGKSFRKMISLCLQKDPEKRPTAAELLRHKFFQKAKNKEFLQEKTLQRAPTISERAKKVRRVPGSSGRLHKTEDGGWEWSDDEFDEESEEGKAAISQLRSPRVKESISNSELFPTTDPVGTLLQVPEQISAHLPQPAGQIATQPTQVSLPPTAEPAKTAQALSSGSGSQETKIPISLVLRLRNSKKELNDIRFEFTPGRDTAEGVSQELISAGLVDGRDLVIVAANLQKIVEEPQSNRSVTFKLASGVEGSDIPDDGKLIGFAQLSIS

SEQ ID NO: 58 - protein, 560 aa, Saccharomyces cerevisiae
MVLLKEPVQPLPRSSLLYNNASNSSSRIKETRKVKLLYNPLTKRQILNNFEILATLGNGQYGKVKLARDLGTGALVAIKILNRFEKRSGYSLQLKVENPRVNQEIEVMKRCHHENVVELYEILNDPESTKVYLVLEYCSRGPVKWCPENKMEIKAVGPSILTFQQSRKVVLDVVSGLEYLHSQGITHRDIKPSNLLISSNGTVKISDFGVAMSTATGSTNIQSSHEQLLKSRALGTPAFFAPELCSTEKEYSCSSAIDIWSLGVTIYCLLFGKLPFNANSGLELFDSIINKPLEFPSYEEMLNGATSGITMEEYTDAKDLLKKLLQKDPDKRIKLADIKVHPFMCHYGKSDAASVLTNLETFHELKVSPPSSCKRVELVSLPVNSSFASLDSVYMENFDHNNLRTGADRNSTYSPSIYDANTLSPSAYHNIGSRESSYSSFSSFTSSTAFASQISIQDAPAIGDQQCLIGESGSSLRVNSCEFPQYTTMSPVGEYPFESTEASLSSTLTPVGNVPQRIKAHLVEGKSNSKDDLRIEADASLVFEASDAQRTRRRMSLYKL

SEQ ID NO: 59 - protein, 1142 aa, Saccharomyces cerevisiae
MDRSDKKVNVEEVNVPSNLQIELEKSGTSSSVSLRSPTKSSATNLAGMAEGARDNASIASSSVDSLNMLLERQRVRQLNHPQHQQHISSSLAKTPTTTSSFCSSGSSKNKVKETNRISLTYDPVSKRKVLNTYEIIKELGHGQHGKVKLARDILSKQLVAIKIVDRHEKKQRKFFTFIKSSKISENDKIKREIAIMKKCHHKHVVQLIEVLDDLKSRKIYLVLEYCSRGEVKWCPPDCMESDAKGPSLLSFQETREILRGVVLGLEYLHYQGIIHRDIKPANLLISGDGTVKISDFGVSLAASSTNSSDSSESLDELELAKTVGTPAFFAPEMCLGEDAFTRYNLTKENLFRGSCISFMIDIWAVGVTLYCLLFGMLPFFSDFELKLFEKIVNDPLKFPTFKEIQSNKVSKVSCEEEYEMAKDLLLKLLEKNPQKRMTIPAIKKHPFVSWDFDHVPENDEKLLSSVLEQKLRFQCNQTDQFEPISISKHELKNAVSGVGKKIKESVLKSIPLKDPSDLSNKNYLHPTETTRGRGDANVIVSEGSVLSNIKELSANDGCLNTDSDTNININDDDHYSGDDNDGHLTKRELERELNKFDDKHEAGNMVNLPINSSFASLDSFYIDNFAMARMGMSSPEAGDSVSSVPNLPSAPSSTRLGRSPVFSGVTNQPSPIRPVLPQQKSSFCATGRYDKSHNSLLRNSSSHLTSYNSGRPSSRTGRMNSRNQNLPKIPNSLSKISTTKLTELRVPKDSEIPSPAKNPNADRLRRFPVKKNTKTPAIKDPPRININSSDKSGSKNSPIKSLYQRMKQSKDNSKTFEVRRGNFFSHFNGDDDDSSSQSSVTSSGSESDSELSSTSSSCTSGTQSRNSSNNNAYSETESLPFEFGVDSEDGSGVLLRDLPNEDQIRPFLDIQPCRRMKVKSSLNLEPPSVSSSSSSSSDEDELILNVGTAGHRRRHNSSKLSELSNSPQKGSNNFMYSNGSVHDSETTITPQNMDDLTLHQALSRSQPISKPGPLVLPKRLDQKKATTETSNLTDIVEFNGNNDHRKDKNFDKVLYSRDLLKDALSSTNAGRRRSIPSNKIRGRKDASITMSTNVGNDEHARNTSCHGDKGQENGAIKQRTHERSRSLTVAELNEEKRRSALP

SEQ ID NO: 60 - protein, 640 aa, Saccharomyces cerevisiae
MSPRQLIPTLIPEWAPLSQQSCIREDELDSPPITPTSQTSSFGSSFSQQKPTYSTIIGENIHTILDEIRPYVKKITVSDQDKKTINQYTLGVSAGSGQFGYVRKAYSSTLGKVVAVKIIPKKPWNAQQYSVNQVMRQIQLWKSKGKITTNMSGNEAMRLMNIEKCRWEIFAASRLRNNVHIVRLIECLDSPFSESIWIVTNWCSLGELQWKRDDDEDILPQWKKIVISNCSVSTFAKKILEDMTKGLEYLHSQGCIHRDIKPSNILLDEEEKVAKLSDFGSCIFTPQSLPFSDANFEDCFQRELNKIVGTPAFIAPELCHLGNSKRDFVTDGFKLDIWSLGVTLYCLLYNELPFFGENEFETYHKIIEVSLSSKINGNTLNDLVIKRLLEKDVTLRISIQDLVKVLSRDQPIDSRNHSQISSSSVNPVRNEGPVRRFFGRLLTKKGKKKTSGKGKDKVLVSATSKVTPSIHIDEEPDKECFSTTVLRSSPDSSDYCSSLGEEAIQVTDFLDTFCRSNESLPNLTVNNDKQNSDMKTDRSESSSHSSLKIPTPIKAMIRLKSSPKENGNRTHINCSQDKPSSPLMDRTVGKRTVNNSGARKLAHSSNILNFKAYINSEDSDIRETVEDVKTYLNFADNGQI

SEQ ID NO: 61 - protein, 545 aa, Homo sapiens
MSSCVSSQPSSNRAAPQDELGGRGSSSSESQKPCEALRGLSSLSIHLGMESFIVVTECEPGCAVDLGLARDRPLEADGQEVPLDTSGSQARPHLSGRKLSLQERSQGGLAAGGSLDMNGRCICPSLPYSPVSSPQSSPRLPRRPTVESHHVSITGMQDCVQLNQYTLKDEIGKGSYGVVKLAYNENDNTYYAMKVLSKKKLIRQAGFPRRPPPRGTRPAPGGCIQPRGPIEQVYQEIAILKKLDHPNVVKLVEVLDDPNEDHLYMVFELVNQGPVMEVPTLKPLSEDQARFYFQDLIKGIEYLHYQKIIHRDIKPSNLLVGEDGHIKIADFGVSNEFKGSDALLSNTVGTPAFMAPESLSETRKIFSGKALDVWAMGVTLYCFVFGQCPFMDERIMCLHSKIKSQALEFPDQPDIAEDLKDLITRMLDKNPESRIVVPEIKILVKTMIRKRSFGNPFEGSRREERSLSAPGNLLTKKPTRECESLSELKEARQRRQPPGHRPAPRGGGGSALVRGSPCVESCWAPAPGSPARMHPLRPEEAMEPE

SEQ ID NO: 62 - protein, 243 aa, Artificial
  Consensus for figure 12
PSSSSSSRIKTVLYPLTKRQILNNYILGGQYGKVKLADTLVAIKILKKKKYKDRVKEIVMKRLHHNVVLIEVLDDPSKVYLVLEYCSGVWCMEIVPILSQARVVDVVGLEYLHSQGIIHRDIKPSNILISDGTVKISDFGVTSDSLRVGTPAFAPELCYFIDIWSLGVTLYCLLFGLPFADLLFDKIILFPEMEELKDLLKKLLENKNPKRILIKHPFVDHPDVLTELKPLRVEPVSLKSSLG

SEQ ID NO: 63 - protein, 25 aa, Homo sapiens
DFGFAKRVKGRTWTLCGTPEYLAPE

SEQ ID NO: 64 - protein, 28 aa, Homo sapiens
DFGMCKEHMMDGVTTRTFCGTPDYIAPE

SEQ ID NO: 65 - protein, 27 aa, Homo sapiens
DFGLSNLYQKDKFLQTFCGSPLYASPE

SEQ ID NO: 66 - protein, 26 aa, Homo sapiens
DFGLSNYHQGKFLQTFCGSPLYASPE

SEQ ID NO: 67 - protein, 27 aa, Homo sapiens
DFGMASLQVGDSLLETSCGSPHYACPE

SEQ ID NO: 68 - protein, 27 aa, Homo sapiens
DFGMASLQVGDSLLETSCGSPHYACPE

SEQ ID NO: 69 - protein, 27 aa, Homo sapiens
DFGFGNFYKSGEPLSTWCGSPPYAAPE

SEQ ID NO: 70 - protein, 27 aa, Homo sapiens
DFGFGNFFKSGELLATWCGSPPYAAPE

SEQ ID NO: 71 - protein, 27 aa, Arabidopsis thaliana
DFGLSNIMRDGHFLKTSCGSPNYAAPE

SEQ ID NO: 72 - protein, 27 aa, Arabidopsis thaliana
DFGLSNVMRDGHFLKTSCGSPNYAAPE

SEQ ID NO: 73 - protein, 27 aa, Homo sapiens
DFGLSNMMSDGEFLRTSCGSPNYAAPE

SEQ ID NO: 74 - protein, 27 aa, Homo sapiens
DFGLSNMMSDGEFLRTSCGSPNYAAPE

SEQ ID NO: 75 - protein, 27 aa, Saccharomyces cerevisiae
DFGLSNIMTDGNFLKTSCGSPNYAAPE

SEQ ID NO: 76 - protein, 27 aa, Homo sapiens
DFGFSNLFTPGQLLKTWCGSPPYAAPE

SEQ ID NO: 77 - protein, 29 aa, Homo sapiens
DFGLCAKPKGNKDYHLQTCCGSLAYAAPE

SEQ ID NO: 78 - protein, 21 aa, Artificial
  consensus from figure 19
DFGLSNLGFLTSCGSPYAAPE

SEQ ID NO: 79 - protein, 27 aa, Homo sapiens
DFGFSNEFTVGNKLDTFCGSPPYAAPE

SEQ ID NO: 80 - protein, 27 aa, Homo sapiens
DFGFSNEFTVGNKLDTFCGSPPYAAPE

SEQ ID NO: 81 - protein, 27 aa, Homo sapiens
DFGFSNEFTVGGKLDTFCGSPPYAAPE

SEQ ID NO: 82 - protein, 27 aa, Homo sapiens
DFGFSNEFTLGSKLDTFCGSPPYAAPE

SEQ ID NO: 83 - protein, 23 aa, Homo sapiens
LSNLYHQGKFLQTFCGSPLYRRR

SEQ ID NO: 84 - protein, 23 aa, Homo sapiens
FGNFYKSGEPLSTWCGSPPYRRR

SEQ ID NO: 85 - protein, 23 aa, Homo sapiens
LSNMMSDGEFLRTSCGSPNYRRR

SEQ ID NO: 86 - protein, 23 aa, Homo sapiens
MASLQVGDSLLETSCGSPHYRRR

SEQ ID NO: 87 - protein, 23 aa, Homo sapiens
FSNEFTVGGKLDTFCGSPPYRRR

SEQ ID NO: 88 - protein, 23 aa, Homo sapiens
AKPKGNKDYHLQTCCGSLAYRRR

SEQ ID NO: 89 - protein, 32 aa, Homo sapiens
IAAFGASLQVGDSLLETSCGSPHYACPERVIR

SEQ ID NO: 90 - protein, 25 aa, Homo sapiens
SLQVGDSLLETSCGSPHYACPEVIR

SEQ ID NO: 91 - protein, 20 aa, Homo sapiens
FLQTFCGSPLYASPEIVNGK

SEQ ID NO: 92 - protein, 19 aa, Homo sapiens
LDTFCGSPPYAAPELFQGK

SEQ ID NO: 93 - protein, 25 aa, Homo sapiens
GNKDYHLQTCCGSLAYAAPELIQCK

SEQ ID NO: 94 - protein, 329 aa, Schizosaccharomyces pombe
MSFLFNKRPKSTQDVVRCLCDNLPKLEINNDKKKSFEEVSKCLQNLRVSLCGTAEVEPDADLVSDLSFQIYQSNLPFLLVRYLPKLEFESKKDTGLIFSALLRRHVASRYPTVDYMLAHPQIFPVLVSYYRYQEVAFTAGSILRECSRHEALNEVLLNSRDFWTFFSLIQASSFDMASDAFSTFKSILLNHKSQVAEFISYHFDEFFKQYTVLLKSENYVTKRQSLKLLGEILLNRANRSVMTRYISSAENLKLMMILLRDKSKNIQFEAFHVFKLFVANPEKSEEVIEILRRNKSKLISYLSAFHTDRKNDEQFNDERAFVIKQIERL

SEQ ID NO: 95 - protein, 399 aa, Saccharomyces cerevisiae
MFKKYKNQDLDMAFWWKKNPKTPSDYARLIIEQLNKFSSPSLTQDNKRKVQEECTKYLIGTKHFIVGDTDPHPTPEAIDELYTAMHRADVFYELLLHFVDLEFEARRECMLIFSICLGYSKDNKFVTVDYLVSQPKTISLMLRTAEVALQQKGCQDIFLTVGNMIIECIKYEQLCRIILKDPQLWKFFEFAKLGNFEISTESLQILSAAFTAHPKLVSKEFFSNEINIIRFIKCINKLMAHGSYVTKRQSTKLLASLIVIRSNNALMNIYINSPENLKLIMTLMTDKSKNLQLEAFNVFKVMVANPRKSKPVFDILVKNRDKLLTYFKTFGLDSQDSTFLDEREFIVQEIDSLPRIISSTTEVSNNNASSSNVASITSPSSVMNNQSSILTHSTSPDSR

SEQ ID NO: 96 - protein, 343 aa, Arabidopsis thaliana
MRGLFKSKPRTPADIVRQTRDLLLYADRSNSFPDLRESKREEKMVELSKSIRDLKLILYGNSEAEPVAEACAQLTQEFFKADTLRRLLTSLPNLNLEARKDATQVVANLQRQQVNSRLIAADYLESNIDLMDFLVDGFENTDMALHYGTMFRECIRHQIVAKYVLDSEHVKKFFYYIQLPNFDIAADAAATFKELLTRHKSTVAEFLIKNEDWFFADYNSKLLESTNYITRRQAIKLLGDILLDRSNSAVMTKYVSSMDNLRILMNLLRESSKTIQIEAFHVFKLFVANQNKPSDIANILVANRNKLLRLLADIKPDKEDERFDADKAQVVREIANLKLRELA

SEQ ID NO: 97 - protein, 8 aa, Artificial
  FLAG peptide
DYKDDDDK

SEQ ID NO: 98 - protein, 15 aa, Artificial
  Bovine MBP fragment
GHHAARTTHYGSLPQ

SEQ ID NO: 99 - DNA, 73 bp, Artificial
  PCR primer
ggatccgccaccatggagcagaagctgatctctgaagaggacttgccgttcccgtttgggaagtctcacaaat

SEQ ID NO: 100 - DNA, 34 bp, Artificial
  PCR primer
ggatccttaagcttcttgctgagctggtctcttc

SEQ ID NO: 101 - DNA, 78 bp, Artificial
  PCR primer
caccggatccgccaccatggagcagaagctgatctctgaagaggacttgcctttgtttagtaaatcacacaaaaatcc

SEQ ID NO: 102 - DNA, 34 bp, Artificial
  PCR primer
ggatcctcaaggggccgttttcttcaagtctcgg

SEQ ID NO: 103 - DNA, 69 bp, Artificial
  PCR primer
ggatccgccaccatggactacaaggacgacgatgacaagtcatttcttgtaagtaaaccagagcgaatc

SEQ ID NO: 104 - DNA, 35 bp, Artificial
  PCR prime
ggatcctcagaactcccaatcgtccacctccagct

SEQ ID NO: 105 - DNA, 68 bp, Artificial
  PCR prime
ggatccgccaccatggactacaaggacgacgatgacaagtctcttttggattgcttctgcacttcaag

SEQ ID NO: 106 - DNA, 36 bp, Artificial
  PCR primer
ggatccctagaattcccagtatgagtctttttcatc

SEQ ID NO: 107 - DNA, 66 bp, Artificial
  PCR primer
actagtgccaccatggactacaaggacgacgatgacaagaagctcatcggcaagtacctgatgggg

SEQ ID NO: 108 - DNA, 36 bp, Artificial
  PCR primer
actagttcagtcctccaggtagggcactacagtcat

SEQ ID NO: 109 - protein, 14 aa, Homo sapiens
CDPMKRATIKDIRE

SEQ ID NO: 110 - protein, 22 aa, Homo sapiens
LSNMMSDGEFLRTSCGSPNRRR

SEQ ID NO: 111 - protein, 13 aa, Rattus rattus
KFLRTSCGSPNYA

SEQ ID NO: 112 - DNA, 25 bp, Homo sapiens
actgcagccctggagcccaggaagc

SEQ ID NO: 113 - DNA, 30 bp, Homo sapiens
ctagttgagcttgctgcagatctccagcgc

SEQ ID NO: 114 - DNA, 69 bp, Homo sapiens
actagtgccaccatgtacccatacgatgtgccagattacgccgaaggggccgccgcgcctgtggcgggg

SEQ ID NO: 115 - DNA, 30 bp, Homo sapiens; identical to SEQ ID NO: 113

SEQ ID NO: 116 - DNA, 69 bp, Homo sapiens
actagtgccaccatgtacccatacgatgtgccagattacgccgagtcgctggttttcgcgcggcgctcc

SEQ ID NO: 117 - DNA, 33 bp, Homo sapiens
tcaggtgagctttgagcagaccctcagtgcctg

SEQ ID NO: 118 - DNA, 55 bp, Homo sapiens
gcgtcgactacccatacgatgtgccagattacgccgtcatggcggatggcccgag

SEQ ID NO: 119 - DNA, 55 bp, Homo sapiens
gcactagttacccatacgatgtgccagattacgccgtcatggcggatggcccgag

SEQ ID NO: 120 - DNA, 36 bp, Homo sapiens
gagcggccgctaattcaccaggacatacccgttgtg

SEQ ID NO: 121 - DNA, 60 bp, Homo sapiens
gcggatcctacccatacgatgtgccagattacgccgttatcatgtcggagttcagcgcgg

SEQ ID NO: 122 - DNA, 34 bp, Homo sapiens
gagcggccgctcactgcaccaggacaaacgtgcc

SEQ ID NO: 123 - DNA, 75 bp, Homo sapiens
gcggatcctacccatacgatgtgccagattacgccaaagattatgatgaacttctcaaatattatgaattacatg

SEQ ID NO: 124 - DNA, 41 bp, Homo sapiens
gtgcggccgcttataccttgcagctagataggatgtcttcc

SEQ ID NO: 125 - DNA, 69 bp, Homo sapiens
ccacccccacccaccccagcacgcccaatatgtgggcccctatcggctggagaagacgctgggcaaagg

SEQ ID NO: 126 - DNA, 31 bp, Homo sapiens
cgatgcagcctctcgcggtccctgaagcagc

SEQ ID NO: 127 - DNA, 31 bp, Homo sapiens
gctgcttcagggaccgcgagaggctgcatcg

SEQ ID NO: 128 - DNA, 28 bp, Homo sapiens
tcagggcagaggggtcccgttggtggcc

SEQ ID NO: 129 - DNA, 67 bp, Homo sapiens
ggtgggggctctcccgcctaccacctcccccacccccacccccacccaccccagcacgcccaatatg

SEQ ID NO: 130 - DNA, 76 bp, Homo sapiens
ggatcctacccatacgatgtgccagattacgcctcgtccggggccaaggagggaggtgggggctctcccgcctacc

SEQ ID NO: 131 - DNA, 25 bp, Homo sapiens
gcggatcctacccatacgatgtgcc

SEQ ID NO: 132 - DNA, 69 bp, Homo sapiens
ggatccgccaccatgtacccatacgatgtgccagattacgccacatcgacggggaaggacggcggcgcg

SEQ ID NO: 133 - DNA, 57 bp, Homo sapiens
gcggccgctcagaggctactctcgtagctggtggccaccttctggcccttaagccca

SEQ ID NO: 134 - DNA, 90 bp, Homo sapiens
ggagccgggcccgcgggccgcctgctgcctccgcccgcgccggggtccccagccgcccccgctgccgtgtcccctgcggccggccagccg

SEQ ID NO: 135 - DNA, 41 bp, Homo sapiens
tgaagaggttactgaaaccaaaatctgctattttgatattc

SEQ ID NO: 136 - DNA, 87 bp, Homo sapiens
gattacgccgcggcggcggcggcgagcggagctggcggggctgccggggccgggactgggggagccgggcccgcgggccgcctgctg

SEQ ID NO: 137 - DNA, 55 bp, Homo sapiens
gcggatcctacccatacgatgtgccagattacgccgcggcggcggcggcgagcgg

SEQ ID NO: 138 - DNA, 90 bp, Homo sapiens
atagcagattttggtttcagtaacctcttcactcctgggcagctgctgaagacctggtgtggcagccctccctatgctgcacctgaactc

SEQ ID NO: 139 - DNA, 34 bp, Homo sapiens
ctgtggacataaaaaatgggatgcggaactttcc

SEQ ID NO: 140 - DNA, 34 bp, Homo sapiens
ggaaagttccgcatcccattttttatgtccacag

SEQ ID NO: 141 - DNA, 33 bp, Homo sapiens
gagcggccgcttacacgcctgcctgctccatgc

SEQ ID NO: 142 - DNA, 54 bp, Homo sapiens
gcgaattctacccatacgatgtgccagattacgcctcggcccggacgccattgc

SEQ ID NO: 143 - DNA, 37 bp, Homo sapiens
catcatacttctgatttattaaggcatcatttatttc

SEQ ID NO: 144 - DNA, 37 bp, Homo sapiens
gaaataaatgatgccttaataaatcagaagtatgatg

SEQ ID NO: 145 - DNA, 41 bp, Homo sapiens
gagtcgacttacagcttaagctcatttgctatttttgatgc

SEQ ID NO: 146 - DNA, 54 bp, Homo sapiens
gcggtacctacccatacgatgtgccagattacgcctcggcccggacgccattgc

SEQ ID NO: 147 - DNA, 43 bp, Homo sapiens
gagcggccgcttacagcttaagctcatttgctatttttgatgc

SEQ ID NO: 148 - DNA, 56 bp, Homo sapiens
gcgtcgactacccatacgatgtgccagattacgccattcggggccgcaactcagcc

SEQ ID NO: 149 - DNA, 56 bp, Homo sapiens
gcactagttacccatacgatgtgccagattacgccattcggggccgcaactcagcc

SEQ ID NO: 150 - DNA, 39 bp, Homo sapiens
gagcggccgcttaaagcttcagctcgttggctattttgg

SEQ ID NO: 151 - DNA, 73 bp, Homo sapiens
gcggccgcagccaccatgtacccatacgatgtgccagattacgcctccactaggaccccattgccaacggtga

SEQ ID NO: 152 - DNA, 41 bp, Homo sapiens
gcggccgcttacagctttagctcattggcaattttggaagc

SEQ ID NO: 153 - DNA, 68 bp, Homo sapiens
agatctgccaccatgtacccatacgatgtgccagattacgcctcttcgcggacggtgctggccccggg

SEQ ID NO: 154 - DNA, 25 bp, Homo sapiens
tgccctgaaacagctccggggcggc

SEQ ID NO: 155 - DNA, 25 bp, Homo sapiens
gggatcgaagctggacacgttctgc

SEQ ID NO: 156 - DNA, 39 bp, Homo sapiens
gcggccgctcacactccaggggaatcggagcagccgggg

SEQ ID NO: 157 - protein, 15 aa, Artificial
  Artificial AMPK kinase substrate
AMARAASAAALARRR

SEQ ID NO: 158 - protein, 23 aa, Homo sapiens
LSNLYHQGKFLQTFCGSPLYRRR